(19) United States
(12) Patent Application Publication
(10) Pub. No.: US 2017/0246288 A1
(43) Pub. Date: Aug. 31, 2017

(54) MULTILAMELLAR LIPID VESICLE COMPOSITIONS AND METHODS OF USE

(71) Applicant: Vedantra Pharmaceuticals, Inc., Cambridge, MA (US)

(72) Inventors: Adrienne LI, Cambridge, MA (US); Jackson EBY, Arlington, MA (US); Peter C. DEMUTH, Medford, MA (US)

(73) Assignee: Vedantra Pharmaceuticals, Inc., Cambridge, MA (US)

(21) Appl. No.: 15/510,290

(22) PCT Filed: Sep. 11, 2015
(86) PCT No.: PCT/US15/49819

§ 371 Date: Mar. 10, 2017

Related U.S. Application Data
(60) Provisional application No. 62/049,239, filed on Sep. 11, 2014.
(60) Provisional application No. 62/049,286, filed on Sep. 11, 2014.
(60) Provisional application No. 62/104,321, filed on Jan. 16, 2015.
(60) Provisional application No. 62/104,282, filed on Jan. 16, 2015.

(51) Int. Cl. A61K 39/12; A61K 31/56; A61K 35/76; C07K 16/30; A61K 39/00; A61K 38/16

(52) U.S. Cl. CPC A61K 39/12; A61K 39/0011; A61K 38/162; A61K 35/76; C07K 16/30; A61K 31/56; C12N 2710/00032; C07K 2319/00; A61K 2039/525; A61K 2300/00

(57) ABSTRACT

The present invention provides novel and inventive drug delivery systems with higher loading capability, a capacity to sequester high tumors levels of both hydrophobic and hydrophilic agents simultaneously, and longer release profiles. Some aspects of these delivery systems include compositions including stabilized multilamellar lipid vesicles having crosslinked lipid bilayers (referred to herein as inter-bilayer-crosslinked multilamellar vesicles or ICMV) covalently conjugated to an agent (e.g., an antigen).

### BACKGROUND OF THE INVENTION

Liposomes have been widely used as a delivery vehicle for small molecules; however, it remains difficult to achieve high levels of encapsulation for many macromolecular drugs within liposomes and many drug formulations leak from liposomes too quickly to maintain useful drug delivery kinetics. While drug delivery by micro- and nanoparticles can encapsulate proteins and small-molecule drugs, this still typically yields very low total mass encapsulated drug per mass of particles, typically on the order of about 10 μg drug/mg particles. In addition, the organic solvents used in polymer particle synthesis and hydrophobic/acidic environment within these particles can lead to destruction of therapeutics. (See Zhu et al. Nat. Biotechnol. 2000 18:52-57.)

One area that can benefit from effective drug delivery by micro- and nanoparticles is human papilloma virus (HPV)-related cancer. HPV-related cancer is one of the fastest growing cancers in the world. Overall, 5% of all cancers world-wide can be attributed to HPV infections. HPV-related cancer is the second leading cause of cancer-related deaths in women and is a leading cause of a growing number of cervical, oropharyngeal, and anal cancers. In particular, strains 16 and 18 of HPV are estimated to be responsible for 70% of cervical cancer cases, leading to about 500,000 new cases and 270,000 deaths world-wide each year. There are about a dozen other high-risk HPV strains that collectively account for the other 30% of cervical cancers. HPV is spread by skin-to-skin sexual contact. The Centers for Disease Control estimates that at least half of all sexually active individuals will acquire HPV at some point in their lives, whereas at least 80% of women will acquire an HPV infection by age 50.

Early detection of HPV infection is extremely rare due to the lack of symptoms. The FDA-approved HPV vaccine is recommended for all men and women, especially for girls aged 11 to 12 with catch-up vaccination for women up to age 26. However, a 2010 survey in the U.S. found that the rates of complete HPV immunization are extremely low, only 11% of 13-17 year olds had received the complete three series of HPV vaccine injections, while only 25% had received at least one dose.

Furthermore, available prophylactic vaccines often prevent but cannot cure existing cancers and subunit vaccines to date fail to produce essential cellular immunity. Prior cancer vaccines based on recombinant proteins avoided toxicity and anti-vector immunity associated with live vaccine (e.g., viral) vectors, but their immunogenicity was poor, particularly for CD8⁺ T-cell (CD8T) responses. Synthetic particles carrying antigens and adjuvant molecules have been developed to enhance subunit vaccines, but in general these materials have failed to elicit CD8T responses comparable to live vectors in preclinical animal models.

Both the lack of symptoms and the low rates of complete HPV immunization contribute to the extreme widespread of HPV infections, which eventually cause HPV-related cancers. Early clinical manifestations of HPV infection include anogenital warts, recurrent respiratory papillomatosis, and cervical cancer precursors (cervical intraepithelial neoplasia), some of which can be treated by, e.g., surgery. However, by the time that HPV infection has shown one or more of these symptoms, the risk of developing HPV-related cancer, such as cervical, anal, vaginal, vulvar, penile, or oropharyngeal cancer, becomes extremely high. Currently, the only treatment for HPV-induced cancers is chemoradiation,

There exists a need for novel and effective immunotherapy against HPV-induced cancer, such as cervical cancer.

### SUMMARY OF THE INVENTION

The invention provides novel and inventive drug delivery systems with higher loading capability, a capacity to sequester high levels of both hydrophobic and hydrophilic agents simultaneously, and longer release profiles. Some aspects of these delivery systems include compositions including stabilized multilamellar lipid vesicles having crosslinked lipid bilayers (referred to herein as interbilayer-crosslinked multilamellar vesicles or ICMV) covalently conjugated to an agent, such as an antigen (e.g., a mutant human papilloma virus (HPV) peptide. The agent covalently conjugated to an ICMV of the present invention is not a terminal-cysteine-bearing antigen as defined in U.S. Patent Publication No. US2012/0177724, e.g., at p. 8, ¶ [0091], lines 8-30.

The vesicles of the invention have unexpected enhanced encapsulation efficiency, and they are able to release encapsulated (or otherwise entrapped) agents via slow and sustained kinetics even in the presence of serum, making them highly desirable as sustained delivery vehicles in vivo. Moreover, as described in greater detail herein, the vesicles of the invention may be synthesized in aqueous environments, thereby avoiding the harsh conditions that are common in various prior art methods including the use of organic solvents and/or acidic environments. As a result, these synthesis methods are more suitable for a variety of agents including those that would typically be compromised structurally and/or functionally using such prior art methods. The resultant vesicles are therefore free of organic solvent and may be comprised solely of the lipids, including biodegradable lipids, and any agent encapsulated therein or therethrough.

The invention is based in part on these and other surprising and unexpected characteristics of the vesicles of the invention, as described in greater detail herein. Accordingly, the invention provides these stabilized vesicles, compositions including these vesicles, methods of making these vesicles, and methods of use thereof.

In a first aspect, the invention features a composition including: (a) a multilamellar lipid vesicle having crosslinks between lipid bilayers; and (b) one or more mutant human papilloma virus (HPV) peptides; wherein at least one of the one or more mutant HPV peptides is conjugated to a lipid of the multilamellar lipid vesicle.

In some embodiments, the mutant HPV peptide has at least one amino acid substitution relative a wild-type HPV peptide sequence. In some embodiments, the mutant HPV peptide has two to six amino acid substitutions relative a wild-type HPV peptide sequence.

In some embodiments, the mutant HPV peptide is derived from a HPV of genotype 16 (HPV-16) or 18 (HPV-18). In some embodiments, the mutant HPV peptide is derived from a HPV E6 protein or a HPV E7 protein.

In some embodiments, the mutant HPV peptide is derived from a HPV-16 E6 protein. In some embodiments, the mutant HPV peptide is derived from a HPV-16 E7 protein. In some embodiments, the mutant HPV peptide is derived from a HPV-18 E6 protein. In some embodiments, the mutant HPV peptide is derived from a HPV-18 E7 protein.

In some embodiments, the mutant HPV peptide includes a fusion protein of a first HPV peptide and a second HPV peptide. In some embodiments, the first HPV peptide and the second HPV peptide are of the same genotypes. In some embodiments, the first HPV peptide and the second HPV peptide are of different genotypes.

In some embodiments, the first HPV peptide and the second HPV peptide are both HPV-16 peptides. In some embodiments, the HPV-16 peptides are HPV-16 E6 peptides or HPV-16 E7 peptides. In some embodiments, the first HPV peptide and the second HPV peptide are both HPV-18 peptides. In some embodiments, the HPV-18 peptides are HPV-18 E6 peptides or HPV-18 E7 peptides.

In some embodiments, the first HPV peptide is a HPV-16 peptide and the second HPV peptide is a HPV-18 peptide. In some embodiments, the HPV-16 peptide is a HPV-16 E6 or HPV-16 E7 peptide. In some embodiments, the HPV-18 peptide is a HPV-18 E6 or HPV-18 E7 peptide.

In some embodiments, the HPV-16 E6 peptide is derived from a HPV-16 E6 protein. In some embodiments, the HPV-18 E6 peptide is derived from a HPV-18 E6 protein. In some embodiments, the HPV-16 E7 peptide is derived from a HPV-16 E7 protein. In some embodiments, the HPV-18 E7 peptide is derived from a HPV-18 E7 protein.

a HPV E6 peptide and a HPV E7 peptide. In some embodiments, the HPV E6 peptide in the fusion protein is derived from a HPV-16 E6 protein or a HPV-18 E6 protein. In some embodiments, the HPV E7 peptide in the fusion protein is derived from a HPV-16 E7 protein or a HPV-18 E7 protein.

In a second aspect, the invention features a composition including: a multilamellar lipid vesicle having crosslinks between lipid bilayers including one or more first mutant HPV peptides, and one or more second mutant HPV peptides, wherein at least one of the first mutant HPV peptides is conjugated to a first lipid of the multilamellar lipid vesicle, wherein at least one of the second mutant HPV peptides is conjugated to a second lipid of the multilamellar lipid vesicle, and wherein the first and second mutant HPV peptides are different. In some embodiments, in addition to the first and second mutant HPV peptides, the multilamellar lipid vesicle having crosslinks between lipid bilayers includes a third, fourth, fifth, sixth, seventh, eighth, ninth, tenth, eleventh, twelfth, and/or thirteenth mutant HPV peptides In some embodiments, the invention features a composition containing a multilamellar lipid vesicle having crosslinks between lipid bilayers and including more than one mutant HPV peptide, e.g., at least two, three, four, five, six, seven, eight, nine, ten, 11, 12, or 13 different mutant HPV peptides. Preferably, the multilamellar lipid vesicle contains no more than 13 different mutant HPV peptides (e.g., no more than two, three, four, five, six, seven, eight, nine, ten, 11, 12, or 13 different mutant HPV peptides).

In some embodiments, the first and/or second mutant HPV peptides are derived from a HPV-16 E6 protein, a HPV-16 E7 protein, a HPV-18 E6 protein, or a HPV-18 E7 protein.

In some embodiments, the HPV-16 E6 protein has the sequence of SEQ ID NO: 1.

In some embodiments, the HPV-16 E7 protein has the sequence of SEQ ID NO: 2.

In some embodiments, the HPV-18 E6 protein has the sequence of SEQ ID NO: 3.

In some embodiments, the HPV-18 E7 protein has the sequence of SEQ ID NO: 4.

In some embodiments, the one or more mutant HPV peptides include one or more functionalized mutant HPV peptides. In some embodiments, the one or more functionalized mutant HPV peptides include thiol functionalized, maleimide functionalized, hydrazine functionalized, azide functionalized, alkyne functionalized, amine functionalized, carboxylic acid functionalized, alkene functionalized, and/or tetrazine functionalized mutant HPV peptides. In some embodiments, the one or more functionalized mutant HPV peptides are maleimide functionalized and/or hydrazine functionalized.

In some embodiments, the conjugated mutant HPV peptide is encapsulated within the vesicle. In some embodiments, the conjugated mutant HPV peptide is encapsulated between lipid bilayers of the vesicle. In some embodiments, the conjugated mutant HPV peptide is present on an outer surface of the vesicle. In some embodiments, the composition includes at least one molecule of the one or more mutant HPV peptides that is not conjugated to a lipid of the multilamellar lipid vesicle.

In some embodiments, the composition described herein further includes an immunomodulator. In some embodiments, the composition described herein further includes an adjuvant.

In a third aspect, the invention features a composition including: (a) a multilamellar lipid vesicle having crosslinks between lipid bilayers; and (b) one or more agents; wherein the one or more agents are not terminal-cysteine-bearing antigens; and wherein at least one molecule of the one or more agents is conjugated to a lipid of the multilamellar lipid vesicle. In some embodiments, the agent includes an internal cysteine.

In a fourth aspect, the invention features a composition including: (a) a multilamellar lipid vesicle having crosslinks between lipid bilayers; and (b) one or more agents; wherein at least one molecule of the one or more agents is conjugated to a lipid of the multilamellar lipid vesicle, and wherein the conjugation does not include a cysteine.

In a fifth aspect, the invention features a composition including: (a) a multilamellar lipid vesicle having crosslinks between lipid bilayers; and (b) one or more agents; wherein at least one molecule of the one or more agents is conjugated to a lipophilic moiety.

In some embodiments, the compositions of the third, fourth, and fifth aspects of the invention further include an adjuvant. In some embodiments, the compositions of the third, fourth, and fifth aspects of the invention further include an immunomodulator.

In some embodiments, the agent is an adjuvant.

In some embodiments, the one or more agents include one or more antigens. In some embodiments, the one or more agents include two or more antigens. In some embodiments, the antigen is a full-length protein antigen. In some embodiments, the antigen is a peptide antigen. In some embodiments, the antigen is a cancer antigen.

In some embodiments, the cancer antigen is a gp100 (Glycoprotein 100). In some embodiments, the cancer antigen is a NY-ESO-1 (esophageal cancer-1). In some embodiments, the cancer antigen is a member of the mucin (MUC) family. In some embodiments, the cancer antigen is MUC1. In some embodiments, the cancer antigen is selected from a group consisting of a MAGE-A1 (melanoma antigen family A1), a MAGE-A2, a MAGE-A3, a MAGE-A4, a MAGE-A5, a MAGE-A6, a MAGE-A7, a MAGE-A8, a MAGE-A9, a MAGE-A10, a MAGE-A11, a MAGE-A12, a MAGE-Xp2 (MAGE-B2), a MAGE-Xp3 (MAGE-B3), a MAGE-Xp4 (MAGE-B4), a MAGE-C1, a MAGE-C2, a MAGE-C3, a MAGE-C4, and a MAGE-05.

In certain embodiments, the cancer antigen may be Wilms' tumor antigen 1 (WT1), MUC1, LMP2 (latent membrane protein 2 from Epstein-Barr virus), EGFRvIII, Her2/neu, Idiotype antigens (see, e.g., Weng et al., J. Clin Oncol. 22:4717-24, 2004), MAGE-A3, non-mutant p53, NY-ESO-1, PSMA (prostate-specific membrane antigen), GD2, CEA (carcinoembryonic antigen), MelanA/MART1, Ras mutants, gp100, mutant p53, Proteinase3 (PR1), BCR-Abl breakpoints (see, e.g., Maslak et al., Leukemia 22:1613-6, 2008), Tyrosinase, Survivin, PSA (prostate-specific antigen), hTERT (human telomerase), sarcoma translocation breakpoints (see, e.g., Mackall et al., Clin Cancer Res. 14:4850-8, 2008), EphA2, PAP (prostatic acid phosphatase), ML-IAP (ML-inhibitor of apoptosis), AFP (alphafetoprotein), EpCAM (epithelial cell adhesion molecule), ERG (TMPRSS2 ETS fusion gene), NA17, PAX3 (paired box 3), Androgen receptor, Cyclin B1, Polysialic acid, MYCN (N-myc), RhoC, TRP-2 (tyrosinase-related protein 2), GD3, Fucosyl GM1, Mesothelin, PSCA (prostate stem cell antigen), MAGE-A1, CYP1B1 (cytochrome P450 1B1), PLAC1 (placenta-specific 1), GM3, BORIS (brother of the regulator of imprinted sites), Tn (N-acetylgalactosamine linked to serine or threonine by a glycosidic bond), GloboH, ETV6-AML, NY-BR-1, RGS5 (regulator of G protein signaling 5), SART3 (squamous cell carcinoma antigen recognized by T cells 3), STn (sialyl Tn antigen), Carbonic anhydrase IX, PAX5 (paired box 5), OY-TES1, Sperm protein 17, LCK (p56 form), HMWMAA (high molecular weight melanoma associated antigen), AKAP-4 (A-kinase anchor protein 4), SSX2 (synovial sarcoma breakpoint 2), XAGE1 (x antigen 1), B7H3, Legumain, Tie 2, Page4, VEGFR2 (vascular endothelial growth factor receptor 2), MAD-CT-1 (melanoma cancer testis antigen-1), FAP (fibroblast activation protein), PDGFR-b (platelet-derived growth factor receptor-b), MAD-CT-2 (melanoma cancer testis antigen-2), or Fos-related antigen 1.

In some embodiments of the third, fourth, and fifth aspects of the invention, the agent is a HPV peptide. In some embodiments, the HPV peptide is derived from a HPV-16 E6 protein, a HPV-16 E7 protein, a HPV-18 E6 protein, or a HPV-18 E7 protein. In some embodiments, the HPV peptide includes a fusion protein of a HPV E6 peptide and a HPV E7 peptide. In some embodiments, the HPV E6 peptide in the fusion protein is derived from a HPV-16 E6 protein or a HPV-18 E6 protein. In some embodiments, the HPV E7 peptide in the fusion protein is derived from a HPV-16 E7 protein or a HPV-18 E7 protein.

In some embodiments of the third, fourth, and fifth aspects of the invention, the one or more agents include one or more functionalized agents. In some embodiments, the one or more functionalized agents include thiol functionalized, maleimide functionalized, hydrazine functionalized, azide functionalized, alkyne functionalized, amine functionalized, carboxylic acid functionalized, alkene functionalized, and/or tetrazine functionalized agents. In some embodiments, the one or more functionalized agents are maleimide functionalized and/or hydrazine functionalized.

In some embodiments of the third, fourth, and fifth aspects of the invention, the conjugated agent is encapsulated within the vesicle. In some embodiments, the conjugated agent is encapsulated between lipid bilayers of the vesicle. In some embodiments, the conjugated agent is present on an outer surface of the vesicle. In some embodiments, the composition includes at least one molecule of the one or more agents that is not conjugated to a lipid.

In some embodiments of all aspects of the invention described herein, at least two lipid bilayers in the multilamellar lipid vesicle are covalently crosslinked to each other through headgroups that react with covalent crosslinkers to form the covalent crosslinks between lipid bilayers. In some embodiments, the lipid bilayers include anionic and/or neutral lipids. In some embodiments, the lipid bilayers include cationic lipids. In some embodiments, the vesicle includes a functionalized lipid. In some embodiments, the functionalized lipid is a maleimide functionalized lipid. In some embodiments, the maleimide functionalized lipid is a maleimide functionalized phosphoethanolamine. In some embodiments, the vesicle includes phosphocholine. In some embodiments, the vesicle includes phosphoglycerol. In some embodiments, the vesicle includes dioleoylphosphatidylcholine (DOPC), dioleoylphosphatidylglycerol (DOPG), and a maleimide functionalized lipid. In some embodiments, the vesicle is conjugated to polyethylene glycol.

In some embodiments of all aspects of the invention described herein, the composition further includes one or more pharmaceutically acceptable carriers. In some embodiments, the composition further includes an excipient suitable for lyophilization. In some embodiments, the excipient suitable for lyophilization includes sucrose.

In a sixth aspect, the invention features a method including administering to a subject in need thereof, a therapeutically effective amount of a composition described herein. In some embodiments, an immunomodulator is not administered to the subject.

In a seventh aspect of the invention, the invention features a method including administering to a subject in need thereof, 1) a therapeutically effective amount of a composition described herein, and 2) one or more immunomodulators, wherein the immunomodulator is not a CT-011 antibody.

In a eighth aspect of the invention the invention features a method including administering to a subject in need thereof, 1) a multilamellar lipid vesicle having crosslinks between lipid bilayers including one or more mutant HPV peptides, and 2) one or more immunomodulators, wherein the immunomodulator is not a CT-011 antibody.

In some embodiments of the sixth and eighth aspects of the invention, 1) and 2) are administered substantially simultaneously. In some embodiments, 1) and 2) are administered separately. In some embodiments, 1) is administered first, followed by administering of 2). In some embodiments, 2) is administered first, followed by administering of 1).

In some embodiments of the sixth and eighth aspects of the invention, the immunomodulator is selected from the group consisting of a PD-1 (programmed cell death protein-1) inhibitor, an anti-CTLA-4 (cytotoxic T-lymphocyte-associated protein 4) antibody, an anti-CD40 (Cluster of differentiation 40) antibody, a cyclophosphamide (CPM), an AMD3100, an anti-LAG-3/CD223 (Lymphocyte Activation Gene 3/Cluster of differentiation 223) antibody, an anti-B7-H5 antibody, an anti-OX40 antibody, an anti-CD28 antibody, an anti-GITR (glucocorticoid-induced TNFR related protein receptor) antibody, an anti-4-1BB/CD137 antibody, a 4-1 BB ligand, an anti-BTLA (B- and T-lymphocyte attenuator) antibody, an anti-TIM-3/HAVCR2 (T cell immunoglobulin and mucin protein 3/Hepatitis A virus cellular receptor 2) antibody, an anti-KIR (Killer cell immunoglobulin-like receptor) antibody, an anti-Flt3/CD135 (Fms-Related Tyrosine Kinase 3/Cluster of differentiation 135) antibody, an anti-FasL antibody, an anti-CD25 antibody, an GM-CSF (Granulocyte-macrophage colony-stimulating factor), an anti-GM-CSF-receptor (R) antibody, an IL-2 (Interleukin-2), an anti-IL-2-R antibody, an IL-7, an anti-IL-7-R antibody, an IL-21, an anti-IL-21-R antibody, an IL-12, an anti-IL-12-R antibody, an IL-15, an anti-IL-15-R antibody, an IL-18, an anti-IL-18-R antibody, an anti-IDO antibody, an ipilimumab, a crizotinib, a ceritinib, a celecoxib, a SOCS-1 (Suppressor of cytokine signaling-1) inhibitor, a heat shock protein (HSP), a HSP inhibitor, and an anti-galectin-1 antibody. In some embodiments, the immunomodulator is selected from the group consisting of a PD-1 inhibitor, an anti-GITR antibody, an anti-CTLA-4 antibody, an anti-CD40 antibody, a cyclophosphamide (CPM), and an AMD3100. In some embodiments, the PD-1 inhibitor is an anti-PD-1 antibody or an anti-PD-L1 antibody.

In some embodiments of the methods described herein, the subject has cancer.

In some embodiments, the cancer is HPV-positive. In some embodiments, the cancer is selected from cervical cancer, anal cancer, vulvar cancer, head and neck cancer, oropharyngeal cancer, penile cancer, vaginal cancer, virally induced cancer, bladder cancer, pancreatic cancer, lung cancer, liver cancer, ovarian cancer, colon cancer, stomach cancer, neuroblastoma, breast cancer, prostate cancer, renal cancer, leukemia, sarcoma, carcinoma, basal cell carcinoma, non-small cell lung carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, sarcoma, melanoma, astrocytoma, oligoastrocytoma, biliary tract cancer, choriocarcinoma, CNS cancer, larynx cancer, small cell lung cancer, non-small cell lung cancer (NSCLC), adenocarcinoma, giant (or oat) cell carcinoma, squamous cell carcinoma, oral cavity cancer, skin cancer, basal cell cancer, squamous cell cancer, testicular cancer, thyroid cancer, uterine cancer, rectal cancer, a cancer of the respiratory system, and a cancer of the urinary system.

In some embodiments, the cancer is a cervical cancer. In some embodiments, the cancer is an anal cancer. In some embodiments, the cancer is a vulvar cancer. In some embodiments, the cancer is a head and neck cancer. In some embodiments, the cancer is an oropharyngeal cancer. In some embodiments, the cancer is a penile cancer. In some embodiments, the cancer is a vaginal cancer. In some embodiments, the cancer is a solid tumor cancer.

In some embodiments of the methods described herein, the subject has an infection. In some embodiments, the infection is a HPV infection.

In some embodiments of the methods described herein, the subject is a mammal. In some embodiments, the mammal is a human.

In an ninth aspect, the invention features a method including (a) contacting a functionalized lipid with a functionalized agent to form liposomes including the lipid conjugated to the agent; (b) contacting the liposomes including lipids conjugated to the agent with a divalent cation to form fused liposomes; and (c) contacting the fused liposomes with a crosslinker to form multilamellar lipid vesicles having crosslinks between lipid bilayers including lipids conjugated to the agent, wherein the functionalized agent is not a terminal-cysteine-bearing antigen.

In a tenth aspect, the invention features kits. In some embodiments, a kit includes: (i) a multilamellar lipid vesicle having crosslinks between lipid bilayers including one or more agents, (ii) one or more immunomodulators, and (iii) instructions for administering (i) and (ii) to a subject having a disease, wherein the agent is not a terminal-cysteine-bearing antigen, and wherein the immunomodulator is not a CT-011 antibody.

In some embodiments, a kit includes: (i) a multilamellar lipid vesicle having crosslinks between lipid bilayers including one or more agents, and (ii) instructions for administering (i) with one or more immunomodulators to a subject having a disease, wherein the agent is not a terminal-cysteine-bearing antigen, and wherein the immunomodulator is not a CT-011 antibody.

In some embodiments, a kit includes: (i) one or more immunomodulators, and (ii) instructions for administering (i) with a multilamellar lipid vesicle having crosslinks between lipid bilayers including one or more agents to a subject having a disease, wherein the agent is not a terminal-cysteine-bearing antigen, and wherein the immunomodulator is not a CT-011 antibody.

In some embodiments of the kits described herein, the agent is a mutant HPV peptide. In some embodiments, the mutant HPV peptide is derived from a HPV-16 E6 protein, a HPV-16 E7 protein, a HPV-18 E6 protein, or a HPV-18 E7 protein. In some embodiments, the mutant HPV peptide includes a fusion protein of a HPV E6 peptide and a HPV E7 peptide.

In some embodiments of the kits described herein, the disease is cancer. In some embodiments, the cancer is HPV-positive. In some embodiments, the cancer is selected from cervical cancer, anal cancer, vulvar cancer, head and neck cancer, oropharyngeal cancer, penile cancer, vaginal cancer, virally induced cancer, bladder cancer, pancreatic cancer, lung cancer, liver cancer, ovarian cancer, colon cancer, stomach cancer, neuroblastoma, breast cancer, prostate cancer, renal cancer, leukemia, sarcoma, carcinoma, basal cell carcinoma, non-small cell lung carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, sarcoma, melanoma, astrocytoma, oligoastrocytoma, biliary tract cancer, choriocarcinoma, CNS cancer, larynx cancer, small cell lung cancer, non-small cell lung cancer (NSCLC), adenocarcinoma, giant (or oat) cell carcinoma, squamous cell carcinoma, oral cavity cancer, skin cancer, basal cell cancer, squamous cell cancer, testicular cancer, thyroid cancer, uterine cancer, rectal cancer, a cancer of the respiratory system, and a cancer of the urinary system.

In some embodiments of the kits described herein, the disease is infection. In some embodiments, the infection is a HPV infection.

In some embodiments of the kits described herein, the subject is a mammal. In some embodiments, the mammal is a human.

### DESCRIPTION OF THE DRAWINGS

FIG. 1 shows the percentage of IFNγ secreting cells among E6/E7-specific CD8 T cells as a measure of immunogenicity induced by E7-ICMV+E6-ICMV or HPV-16 E6+E7 soluble proteins.

FIG. 2 shows the effects of HPV-16 E6+E7 soluble proteins and E7-ICMV+E6-ICMV on tumor size and survival rate in a mouse model for solid tumors.

FIG. 3A shows the percentage of IFNγ secreting cells among E7-specific CD8 T cells as a measure of immunogenicity induced by ICMV monotherapy (E7-ICMV+E6-ICMV), immunomodulator monotherapy (anti-CTLA-4 antibody (aCTLA-4), anti-PD-1 antibody (aPD-1), or anti-PD-L1 antibody (aPD-L1)), or ICMV+immunomodulator combination therapy.

FIG. 3B shows the percentage of IFNγ secreting cells among E6-specific CD8 T cells as a measure of immunogenicity induced by ICMV monotherapy (E7-ICMV+E6-ICMV), immunomodulator monotherapy (anti-CTLA-4 antibody (aCTLA-4), anti-PD-1 antibody (aPD-1), or anti-PD-L1 antibody (aPD-L1)), or ICMV+immunomodulator combination therapy.

FIG. 3C shows the effects of immunomodulator monotherapy and ICMV monotherapy on tumor size in a mouse model for solid tumors.

FIG. 4 shows the effects of E6-ICMV+E7-ICMV with no immunomodulator and with immunomodulator anti-PD-1 antibody on tumor size of large tumors in a mouse model for solid tumors.

FIG. 5 shows the effects of ICMV monotherapy (E7-ICMV+E6-ICMV), immunomodulator cyclophosphamide (CPM) monotherapy, and ICMV+CPM combination therapy on tumor size and survival rate in a mouse model for solid tumors.

FIG. 6 shows the effect of E6-ICMV+E7-ICMV+anti-PD-1 antibody+cyclophosphamide (CPM) combination therapy on tumor size and tumor regrowth in a mouse model for solid tumors.

FIG. 7 shows the effect of E6-ICMV+E7-ICMV+cyclophosphamide (CPM) combination therapy on tumor regression of tumors of different sizes and survival rate in a mouse model for solid tumors.

FIG. 8A shows the effects of ICMV monotherapy (E7-ICMV+E6-ICMV), immunomodulator cyclophosphamide (CPM) monotherapy, and ICMV+CPM combination therapy on weight change in a mouse model for tongue tumors.

FIG. 8B shows the effects of ICMV monotherapy (E7-ICMV+E6-ICMV), immunomodulator cyclophosphamide (CPM) monotherapy, and ICMV+CPM combination therapy on survival rate in a mouse model for tongue tumors.

FIG. 8C shows images of a tongue tumor in a mouse model treated with ICMV+CPM combination therapy.

FIG. 9 shows the effects of ICMV monotherapy (E7-ICMV+E6-ICMV) and ICMV+an indoleamine-2,3-dioxygenase (IDO) inhibitor combination therapy on tumor size in a mouse model for tongue tumors.

FIG. 10 shows the effects of E7-ICMV with no adjuvant and with monophosphoryl lipid A (MPLA) as adjuvant on tumor size in a mouse model for solid tumors.

FIG. 11 shows effects of E6-ICMV+E7-ICMV on tumor size in a mouse model for solid tumors.

FIG. 12 shows the percentage of tetramer positive (H2-D^{b} E7₄₉₋₅₇ (RAHYNIVTF (SEQ ID NO: 11))-containing tetramers) cells among CD8-positive T cells as a measure of immunogenicity induced by E7-ICMV with adjuvants.

FIG. 13 shows the comparison of tumor regression enhanced by E6+E7 ICMV to that enhanced by E7 minimal peptide vaccination.

FIG. 14 shows the comparison of tumor regression enhanced by E6+E7 ICMV to that enhanced by E7 Listeria vaccination.

FIG. 15 shows the effects of ICMV monotherapy (E6-ICMV+E7-ICMV) with no immunomodulator and with anti-CD40 antibody as an immunomodulator on tumor size in a mouse model for solid tumors.

FIG. 16 shows the effects of ICMV monotherapy (E6-ICMV+E7-ICMV), immunomodulator anti-PD-1 antibody monotherapy, ICMV+anti-PD-1 antibody combination therapy, and ICMV+anti-PD-1 antibody+cyclophosphamide (CPM) combination therapy on tumor size in a mouse model for solid tumors.

FIG. 17 shows the percentage of IFNγ secreting cells among E6/E7-specific CD8 T cells as a measure of immunogenicity induced by ICMV monotherapy (E6-ICMV+E7-ICMV), immunomodulator anti-PD-1 antibody monotherapy, ICMV+anti-PD-1 antibody combination therapy, or ICMV+anti-PD-1 antibody+AMD3100 combination therapy.

### DETAILED DESCRIPTION OF THE INVENTION

The invention provides stabilized multilamellar lipid vesicles for use in, inter alia, delivery of agents. Prior vaccines based on recombinant proteins avoided toxicity and anti-vector immunity associated with live vaccine (e.g., viral) vectors, but their immunogenicity was poor, particularly for CD8⁺ T-cell (CD8T) responses. Synthetic particles carrying antigens and adjuvant molecules have been developed to enhance subunit vaccines, but in general these materials have failed to elicit CD8T cell responses comparable to live vectors in preclinical animal models. In contrast to these prior compositions and methods, the invention provides stabilized multilamellar vesicles, such as interbilayer-crosslinked multilamellar vesicles (ICMVs) formed by crosslinking headgroups of adjacent lipid bilayers within multilamellar vesicles in which an agent has been covalently conjugated. These vesicles include protein antigens (e.g., conjugated protein antigens) and, optionally, immunomodulatory molecules (e.g., immunostimulatory molecules), but exhibit rapid release in the presence of endolysosomal lipases. The protein antigens and optional immunomodulatory molecules (e.g., immunostimulatory molecules) may be present within the vesicle core, within the vesicle walls, or on an outer surface of the vesicle. When used to deliver antigen alone or in the presence of adjuvant, the vesicles of the invention form an extremely potent vaccine (e.g., a whole-protein vaccine), eliciting endogenous T-cell comparable to the strongest vaccine vectors. In some embodiments, the agent covalently conjugated to an ICMV of the present invention is not a terminal-cysteine-bearing antigen as defined in U.S. Patent Publication No. US2012/0177724, e.g., at p. 8, ¶ [0091], lines 8-30.

The vesicles are stabilized by internal linking (e.g., crosslinking) of their lipid bilayers. The stabilized nature of these vesicles and covalent conjugation of agents allows them to incorporate higher amounts of agents and to retain such agents over a longer time period, as compared to simple liposomes or lipid coated nano- or microparticles. Their sustained release kinetics, particularly in the presence of serum, make them useful in in vivo delivery of agents for which a slow, steady and prolonged release is desirable or for which slow release in the extracellular environment but rapid release within cells is desirable. The invention contemplates using such vesicles with a number and variety of agents including prophylactic agents, therapeutic agents, and/or diagnostic agents, as described in greater detail herein. The invention therefore provides compositions including the aforementioned vesicles, methods for their synthesis, and methods for their use.

### Stabilized Multilamellar Lipid Vesicles (MLV)

The invention provides MLV that are stabilized by linking adjacent (or apposed) lipid bilayers to one another and include agents covalently conjugated to the vesicle (e.g., within the vesicle core, within the vesicle walls, or on an outer surface of the vesicle). In some embodiments, the invention provides vesicles that further include linking of two monolayers of a single bilayer. As used herein, a multilamellar vesicle is a nano- or microsphere having a shell that is comprised of two or more concentrically arranged lipid bilayers. As used herein, adjacent or apposed lipid bilayers (or lipid bilayer surfaces) refer to bilayers or surfaces that are in close proximity to each other but that are otherwise distinct and typically physically separate. This term does not typically mean the relationship between the two monolayers of a single bilayer.

As used herein, “linking” means two entities stably bound to one another by any physiochemical means. Any linkage known to those of ordinary skill in the art may be employed including covalent or noncovalent linkage, although covalent linkage is preferred. In some important embodiments described herein, covalent linkage between lipid bilayers (e.g., adjacent or apposed lipid bilayers) in MLV is achieved through the use of crosslinkers and functionalized components of the lipid bilayer. The invention however contemplates that linking, including covalent linking, may be effected in other ways. As an example, the invention contemplates methods in which complementary reactive groups reside on components of adjacent bilayer surfaces and linkage between the bilayer surfaces is effected by reacting those groups to each other even in the absence of a crosslinker. Suitable complementary reactive groups are known in the art and described herein.

The interior of the vesicle is typically an aqueous environment, and it may include an agent such as but not limited to a prophylactic, therapeutic or diagnostic agent. In some instances, the vesicles do not include a solid core, such as a solid polymer core (e.g., a synthetic polymer core). Instead, as discussed above, they may have a fluid core including agents of interest. The core may include monomers for polymerization into a hydrogel core in some instances. The vesicles may also be referred to herein as particles, including nano- or microparticles, although it is to be understood that such nano- or micro-particles have the attributes of the stabilized MLVs and interbilayer crosslinked multilamellar lipid vesicles (ICMVs) of the invention.

The vesicles may have a void volume at their core and/or they may include one or more agents in their core and/or between adjacent (or apposing) lipid bilayers. The conjugated agents may be covalently attached to functionalized lipids (e.g., maleimide functionalized lipids) through a reactive group (e.g., a thiol). Non-conjugated agents may be included in the lipid solution during the synthesis process and in this manner are incorporated (e.g., by encapsulation) into the vesicles during synthesis. Lipophilic molecules may also be incorporated directly into the lipid bilayers as the vesicles are formed or molecules with lipophilic tails may be anchored to the lipid bilayers during vesicle formation. The vesicles may be produced in the absence of harsh solvents, such as organic solvents, and as a result they may be able to encapsulate a wide variety of agents including those that would be susceptible to organic solvents.

The amount of agent in the vesicles may vary and may depend on the nature of the agent. As demonstrated in the Examples, 10-500 μg of protein agent per mg of lipid may be incorporated into the vesicles of the invention. In some embodiments, the vesicles may include about 10 μg of agent, about 20 μg of agent, about 50 μg of agent, or about 100 μg of agent, or about 150 μg of agent, or about 200 μg of agent, or about 250 μg of agent, or about 300 μg of agent, or about 325 μg of agent, or about 350 μg of agent, or about 375 μg of agent, or about 400 μg of agent, or about 500 μg of agent, per mg of lipid. In other embodiments, the vesicles may include 10-20 μg of protein agent per mg of lipid, or 15-60 μg of protein agent per mg of lipid, or 50-200 μg of protein agent per mg of lipid, or 100-300 μg of protein agent per mg of lipid, or 200-400 μg of protein agent per mg of lipid, or 300-500 μg of protein agent per mg of lipid. In some embodiments, the agent may be a protein such as a protein antigen.

The vesicles of the invention may also be characterized by their retention profiles. In some embodiments, the vesicles release agent at a rate of about 25% per week when placed in serum containing media (e.g., 10% serum) and maintained at 37° C. In some embodiments, the vesicles release about 25% of agent in the first week and up to about 90% after about 30 days under these conditions. In some embodiments, the vesicles maintain at least 80%, at least 85%, at least 90%, or at least 95% of their agent when stored in buffer (such as PBS+40% sorbitol) at 4° C. for at least 30 days (e.g., at least 60 days, at least 90 day, at least 120 days, at least 150 days, at least 180 days, at least 210 days).

The number of lipid bilayers in each vesicle may vary, with a typical range of at least 2 to about 50, or at least 2 to about 25, or at least 2 to about 15, or at least 2 to about 10, or at least 2 to about 5.

The diameter of the vesicles may vary. In some instances, the vesicles will have a diameter ranging from about 100 to about 500 nm, including from about 125 to about 300 nm, including from about 150 to about 300 nm, including from about 175 to about 275 nm. In some instances, the diameter ranges from about 150 to about 250 nm. It will be understood that, in any preparation of vesicles, there will be heterogeneity between vesicles (e.g., relating to vesicle diameter, number of lipid bilayers, amount of loaded agent).

As used herein, the vesicles of the invention may also be referred to as liposomes (e.g., stabilized multilamellar liposomes or, as discussed below, interbilayer crosslinked multilamellar liposomes). Accordingly, the use of the term “vesicles” is not intended to convey source or origin of the vesicles. The vesicles of the invention are synthetic vesicles (i.e., they are produced in vitro), as will be discussed in greater detail below.

The vesicles may be isolated, intending that they are physically separated in whole or in part from the environment in which they are synthesized. As an example, vesicles including an agent (i.e., their “cargo” or “payload”) may be separated in whole or in part from vesicles lacking agent (i.e., empty vesicles), and may then be referred to as “isolated vesicles.” Separation may occur based on weight (or mass), density (including buoyant density), size, color, or other methods known in the art (e.g., where the cargo of the vesicle is detectable by its energy emission). Centrifugation can be used to separate vesicles of the invention from simple liposomes or MLVs of identical lipid composition that do not have crosslinked bilayers. For example, centrifugation at about 21,000 g for about 5 minutes is sufficient to separate the vesicles of the invention, which pellet, from these other particle types.

### Interbilayer Crosslinked Multilamellar Lipid Vesicles

An example of the stabilized MLV of the invention is the interbilayer crosslinked multilamellar (lipid) vesicles (ICMV). Like the stabilized MLV described above, ICMV are nano- or microspheres having a shell that is comprised of two or more concentrically arranged lipid bilayers that are conjugated to each other as described herein. The number of lipid bilayers in the stabilized multilamellar vesicles, including the ICMV, may vary from about 2-30 (e.g., 2-15, 5-20, 10-30). Accordingly, in various embodiments, the number of layers may be 2, 3, 4, 5, 6, 7, 8, 9, 10, 11, 12, 13, 14, 15 or more. The bilayers are typically comprised of lipids having hydrophilic heads and hydrophobic tails that are arranged in a manner similar to a cell membrane (i.e., with the hydrophilic heads exposed to typically an aqueous environment and the hydrophobic tails buried in the bilayer).

The ICMV are stabilized via crosslinks between their lipid bilayers, and they are therefore referred to as “interbilayer crosslinked” MLV. As used herein, this means that at least two lipid bilayers in the shell of the vesicle are crosslinked to each other. The crosslinked bilayers are typically those that are apposed or adjacent to each other. Most or all of the lipid bilayers in the shell may be crosslinked to their apposing lipid bilayer in the shell. There may be one or more crosslinks between lipid bilayers. Typically, there will be numerous crosslinks between lipid bilayers. The arrangement and positioning of such crosslinks may be random or non-random. The degree of crosslinks (and thus the resultant stability of the vesicles) will depend upon the proportion of functionalized lipids (or other lipid bilayer components) used to make the vesicles and the crosslinking conditions (including, for example, time of incubation of the vesicles with a crosslinker). It will be understood that the higher the proportion of functionalized lipids (or other lipid bilayer components) in the vesicles, the more crosslinks that will be formed, all other factors and parameters being equal. Similarly, the more favorable the conditions towards crosslinking, the greater degree of crosslinking that will be achieved.

In some embodiments of the present invention, the invention features a composition containing a multilamellar lipid vesicle having crosslinks between lipid bilayers and including one or more agents, wherein the one or more agents are not terminal-cysteine-bearing antigens and wherein at least one molecule of the one or more agents is conjugated to a lipid of the multilamellar lipid vesicle. The term “terminal-cysteine-bearing antigen” refers to the terminal-cysteine-bearing antigen as defined in U.S. Patent Publication No. US2012/0177724, e.g., at p. 8, ¶ [0091], lines 8-30, which states, e.g., terminal-cysteine antigens are “antigens that comprise one or more cysteine residues within 10 amino acid residues of the amino terminus (i.e., at amino acid positions 1 through 10) and/or within 10 amino acid residues of the carboxy terminus (i.e., at amino acids (n-10) through n, where n represents the number of amino acid residues in the antigen). Thus, cysteines may occupy positions 1, 2, 3, 4, 5, 6, 7, 8, 9, 10, n-10, n-9, n-8, n-7, n-6, n-5, n-4, n-3, n-2, n-1 and/or n, where 1 represents the residue at the amino terminus and n represents the residue at the carboxy terminus.”

In some embodiments, the invention features a composition containing a multilamellar lipid vesicle having crosslinks between lipid bilayers and including one or more agents, wherein at least one molecule of the one or more agents is conjugated to a lipid of the multilamellar lipid vesicle and wherein the conjugation does not include a cysteine.

In some embodiments, the invention features a composition containing a multilamellar lipid vesicle having crosslinks between lipid bilayers and including one or more agents, wherein at least one molecule of the one or more agents is functionalized with a lipophilic moiety. An agent functionalized with a lipophilic moiety is described further herein. In some embodiments, the lipophilic moiety includes an acyl group. Once the agent is functionalized with a lipophilic moiety, the agent can insert itself into the lipid bilayer of the ICMV using the lipophilic moiety. The lipophilic moiety serves as a hydrophobic anchor. In some embodiments, the agent functionalized with a lipophilic moiety is inserted or embedded into a lipid bilayer of the ICMV. In some embodiments, the agent functionalized with a lipophilic moiety is inserted or embedded into a lipid bilayer of the ICMV and is not conjugated to a lipid bilayer of the ICMV.

In other embodiments, the invention features a composition containing a multilamellar lipid vesicle having crosslinks between lipid bilayers and including one or more agents, wherein at least one molecule of the one or more agents is conjugated to a lipid of the multilamellar lipid vesicle and wherein the conjugation does not include a cysteine that is within 10 (including within 9, 8, 7, 6, 5, 4, 3, or 2) amino acid residues of or at the amino and/or carboxy terminus of the agent (e.g., an antigen).

Examples of agents that can be conjugated to the multilamellar lipid vesicle are described in detail further herein. In some embodiments, the conjugated agent in the multilamellar lipid vesicle having crosslinks between lipid bilayers is an adjuvant. In some embodiments, the conjugated agent in the multilamellar lipid vesicle having crosslinks between lipid bilayers is a HPV peptide (e.g., a mutant HPV peptide).

In some embodiments, the invention features a composition containing a multilamellar lipid vesicle having crosslinks between lipid bilayers and including more than one antigen, e.g., at least two, three, four, five, six, seven, eight, nine, ten, 11, 12, or 13 different antigens, wherein the antigens are not terminal-cysteine-bearing antigens and wherein at least one molecule of the antigens is conjugated to a lipid of the multilamellar lipid vesicle. The term “terminal-cysteine-bearing antigen” refers to the terminal-cysteine-bearing antigen as defined in U.S. Patent Publication No. US2012/0177724, e.g., at p. 8, [0091], lines 8-30. Preferably, the multilamellar lipid vesicle contains no more than 13 different antigens (e.g., no more than two, three, four, five, six, seven, eight, nine, ten, 11, 12, or 13 different antigens).

In some embodiments, the invention features a composition containing a multilamellar lipid vesicle having crosslinks between lipid bilayers and including more than one antigen, e.g., at least two, three, four, five, six, seven, eight, nine, ten, 11, 12, or 13 different antigens, wherein at least one molecule of the antigens is conjugated to a lipid of the multilamellar lipid vesicle and wherein the conjugation does not include a cysteine. Preferably, the multilamellar lipid vesicle contains no more than 13 different antigens (e.g., no more than two, three, four, five, six, seven, eight, nine, ten, 11, 12, or 13 different antigens).

In other embodiments, the invention features a composition containing a multilamellar lipid vesicle having crosslinks between lipid bilayers and including more than one antigen, e.g., at least two, three, four, five, six, seven, eight, nine, ten, 11, 12, or 13 different antigens, wherein at least one molecule of the antigens is conjugated to a lipid of the multilamellar lipid vesicle and wherein the conjugation does not include a cysteine that is within 10 (including within 9, 8, 7, 6, 5, 4, 3, or 2) amino acid residues of or at the amino and/or carboxy terminus of the agent. Preferably, the multilamellar lipid vesicle contains no more than 13 different antigens (e.g., no more than two, three, four, five, six, seven, eight, nine, ten, 11, 12, or 13 different antigens).

Examples of different antigens that can be conjugated to the multilamellar lipid vesicle are described in detail further herein.

In some embodiments, the invention features a composition containing a multilamellar lipid vesicle having crosslinks between lipid bilayers and including one or more mutant human papilloma virus (HPV) peptides, wherein at least one of the one or more mutant HPV peptides is conjugated to a lipid of the multilamellar lipid vesicle.

In some embodiments, the invention features a composition containing a multilamellar lipid vesicle having crosslinks between lipid bilayers and including one or more first mutant HPV peptides, and one or more second mutant HPV peptides, wherein at least one of the first mutant HPV peptides is conjugated to a first lipid of the multilamellar lipid vesicle, wherein at least one of the second mutant HPV peptides is conjugated to a second lipid of the multilamellar lipid vesicle, and wherein the first and second mutant HPV peptides are different.

In some embodiments, the invention features a composition containing a multilamellar lipid vesicle having crosslinks between lipid bilayers and including more than one HPV peptide, e.g., at least two, three, four, five, six, seven, eight, nine, ten, 11, 12, or 13 different HPV peptides (e.g., wild-type and mutant HPV peptides). Preferably, the multilamellar lipid vesicle contains no more than 13 different HPV peptides (e.g., no more than two, three, four, five, six, seven, eight, nine, ten, 11, 12, or 13 different HPV peptides).

Examples of HPV peptides that can be conjugated to the multilamellar lipid vesicle are described in detail further herein.

In some embodiments, a composition described herein further includes one or more immunomodulators and/or one or more adjuvants, wherein the immunomodulator is not a CT-011 antibody.

### Synthesis Methods

An exemplary synthesis method is as follows: Lipids and optionally other bilayer components are combined to form a homogenous mixture. This may occur through a drying step in which the lipids are dried to form a lipid film. The lipids are then combined (e.g., rehydrated) with an aqueous solvent. The aqueous solvent may have a pH in the range of about 6 to about 8, including a pH of about 7. Buffers compatible with vesicle fusion are used, typically with low concentrations of salt (e.g., a 10 mM bis-tris propane (BTP) pH 7.0 buffer). The nature of the buffer may impact the length of the incubation. For example, a buffer such as HEPES may require a longer incubation time as compared to a buffer such as BTP. If the buffer is HEPES, then the incubation times may be about 6-24 hours, or 8-16 hours, or 10-12 hours. If the buffer is BTP, then the incubation times may be shorter including 1-4 hours, or 1-2 hours. Accordingly a variety of aqueous buffers may be used provided that a sufficient incubation time is also used. This step may also include the presence of the agent(s) to be incorporated into the vesicles. The resultant liposomes may then be fluidized, for example, on a Microfluidics LV-1 with 1-4 fluidizer passes (e.g., 1-2 passers) under a pressure of between 10000-40000 psi (e.g., 25000-35000) to collect between 1-13 mL (e.g., 3-4 mL) from the fluidizer. When fluidization is used, unfunctionalized agents are not encapsulated in the resulting vesicles. Alternatively, the liposomes may be incubated with one or more divalent cations in order to fuse them into multilamellar vesicles. Suitable divalent cations include Mg²⁺, Ca²⁺, Ba²⁺, or Sr²⁺. Multivalent or polymeric cations could also be employed for vesicle fusion. Vesicle fusion could also be achieved via the mixing of cationic vesicles with divalent or higher valency anions; an example would be fusion of cationic liposomes with DNA oligonucleotides or DNA plasmids. This may be done under agitation such as sonication or vortexing. If the liposomes were made in the presence of an agent (e.g., a functionalized agent), the MLVs will include the agent in their core, between the concentrically arranged lipid bilayers, and/or on an outer surface. The invention contemplates fusion of liposomes carrying different agents to form MLVs that include such agents.

The resultant MLVs are then incubated with an agent (e.g., a functionalized agent prepared in parallel) under conditions suitable for conjugation of the agent to a lipid. Conjugation may be carried out by incubating for 0.5-48 hours (e.g., 6-24 hours) at room temperature or elevated temperature (e.g., 37° C.) depending the conjugation method used. Following conjugation of an agent, the MLVs are incubated (e.g., for 0.5-6 hours preferably 1-2 hours) with a crosslinker, such as a membrane-permeable crosslinker, and optionally a divalent cation source such as calcium chloride (CaCl₂) (e.g., about 100-1000 μL CaCl₂ per mL total volume, preferably 150-300 μL/mL). The amount of crosslinker will vary depending on the nature of the reactive groups being linked together, for example, about 10-100 μL dithiothrietol (DTT) per mL lipids (e.g., 20-30 μL/mL) may be used if the functionalized lipids are maleimide functionalized lipids. As stated herein, the nature of the crosslinker will vary depending on the nature of the reactive groups being linked together. For example, a dithiol-containing crosslinker such as DTT or (1,4-Di-[3′-(2′-pyridyldithio)-propionamido]butane) may be used to crosslink MLVs comprised of maleimide functionalized lipids (or other functionalized lipid bilayer components), or diazide crosslinkers could be used to crosslink alkyne headgroup lipids via “click” chemistry. These various incubations are all carried out under aqueous conditions at a pH in the range of about 6 to about 8, or about 6.5 to about 7.5, or at about 7. The crosslinking step may be performed at room temperature (e.g., 20-25° C.) or at an elevated temperature including for example up to or higher than 37° C.

The resultant crosslinked vesicles may then be collected (e.g., by centrifugation or other pelleting means), washed and then optionally PEGylated on their outermost or external surface (e.g., as used herein, the vesicles may be referred to “surface-PEGylated” or “surface-conjugated” to PEG) by incubation with a thiol-PEG. The PEG may be of any size, including but not limited to 0.1-10 kDa, 0.5-5 kDa, or 1-3 kDa, such as a 2 kDa PEG-SH. The incubation period may range from about 10 minutes to 6 hours (e.g., 1 to 3 hours), although it may be shorter or longer depending on other conditions such as temperature and concentration. The PEGylation step may be performed at room temperature (e.g., 20-25° C.) or at an elevated temperature including for example up to or higher than 37° C. The vesicles then may be collected (e.g., by centrifugation or other pelleting means) and washed with water or other aqueous buffer. Centrifugation may be performed at 1000-22000×g (e.g., 3000-21000×g) for about 1 to 30 minutes (e.g., 5 to 15 minutes).

The vesicles may then be extruded through a 0.2 μm membrane one to thirty times (e.g., five to fifteen times such as nine times).

The vesicles may be stored at 4° C. in a buffered solution such as but not limited to PBS solutions containing sucrose, free PEG, polysorbate 20 (PS-20), or sorbitol (e.g., 1-80% w/v sorbitol, 35-45% w/v sorbitol, 40% w/v sorbitol) or they may be lyophilized in the presence of suitable cryopreservants and then stored at −20° C. Suitable cryopreservants include those that include sucrose (e.g., a 1-5% sucrose, and preferably about 3% sucrose solution).

Crosslinking could also be achieved by coupling between a reactive group in one bilayer with a complementary reactive group in the adjacent bilayer. For example, fused vesicles containing succinimidyl ester-functionalized lipid (A) headgroups and primary-amine-containing (B) headgroups could achieve crosslinking by in situ reaction between the A and B lipids of adjacent bilayers. A variety of other complementary functionalized lipids familiar to those skilled in the art could be employed in a similar manner.

The molar ratio of functionalized lipid (or other functionalized component of the lipid bilayer) to crosslinker may vary depending on the conditions. In some instances, it may range from about 1 to about 5. In some embodiments, a molar ratio of 2 is sufficient (i.e., the molar ratio of functionalized lipid (or component) to crosslinker is 2:1). For example, a 2:1 molar ratio of maleimide functionalized lipid to DTT may be used to crosslink the lipid bilayers of the vesicles. The incubation time may range from 1 hour to 24 hours, from 2-18 hours, from 2 to 12 hours, or from 2 to 6 hours. In some instances, it may be about 2 hours. In other instances, it may be overnight (e.g., about 12 hours).

The molar % of the functionalized lipid in the vesicles may range from 1% to 100% or from about 10% to about 60% in some instances, or from about 25% to about 55% in some instances. In some instances, the molar % of the functionalized lipid in the vesicles is typically at least 10%, preferably at least 15%, more preferably at least 20%, and even more preferably at least 25%.

Conversely, the non-functionalized lipids may be present at about 0% to 99% as a molar %. More typically, the non-functionalized lipids may be present at about 40%-75% or 40% to 60% as a molar %.

In one embodiment, the vesicles are synthesized using DOPC, DOPG, and maleimide-functionalized DSPE. The ratio of these lipids to each other may vary. The molar % of DOPC may range from 1-50%, the molar % of DOPG may range from 1-50%, and the molar % of the maleimide functionalized lipid may range from 1-80%. In another embodiment, no DOPG is used. Some embodiments of the invention provide vesicles having a DOPC:DOPG:maleimide functionalized lipid ratio of 40:10:50. Some embodiments provide vesicles having a DOPC:DOPG:maleimide functionalized lipid ratio of 60:15:25. Some embodiments provide vesicles comprised of DOPG and a maleimide functionalized lipid.

### Lipids

The vesicles are comprised of one or more lipids. The type, number and ratio of lipids may vary with the proviso that collectively they form spherical bilayers (i.e., vesicles). The lipids may be isolated from a naturally occurring source or they may be synthesized apart from any naturally occurring source.

At least one (or some) of the lipids is/are amphipathic lipids, defined as having a hydrophilic and a hydrophobic portion (typically a hydrophilic head and a hydrophobic tail). The hydrophobic portion typically orients into a hydrophobic phase (e.g., within the bilayer), while the hydrophilic portion typically orients toward the aqueous phase (e.g., outside the bilayer, and possibly between adjacent apposed bilayer surfaces). The hydrophilic portion may include polar or charged groups such as carbohydrates, phosphate, carboxylic, sulfato, amino, sulfhydryl, nitro, and hydroxy groups. The hydrophobic portion may include apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

In some embodiments, the lipids are phospholipids. Phospholipids include without limitation phosphatidylcholine, phosphatidylethanolamine, phosphatidylglycerol, phosphatidylinositol, and phosphatidylserine. It is to be understood that other lipid membrane components, such as cholesterol, sphingomyelin, and cardiolipin may be used.

The lipids may be cationic, anionic, and/or neutral (including zwitterionic and polar) lipids. In some embodiments, the lipids are anionic and/or neutral (e.g., anionic or neutral phospholipids). Neutral lipids exist in an uncharged or neutral zwitterionic form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides and diacylglycerols. Examples of zwitterionic lipids include without limitation dioleoylphosphatidylcholine (DOPC), dimyristoylphosphatidylcholine (DMPC), and dioleoylphosphatidylserine (DOPS). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Collectively, anionic and neutral lipids are referred to herein as non-cationic lipids. Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol.

Additional nonphosphorous containing lipids include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, diacylphosphatidylcholine, diacylphosphatidylethanolamine, ceramide, sphingomyelin, cephalin, and cerebrosides. Lipids such as lysophosphatidylcholine and lysophosphatidylethanolamine may be used in some instances. Noncationic lipids also include polyethylene glycol-based polymers such as PEG 2000, PEG 5000 and polyethylene glycol conjugated to phospholipids or to ceramides (referred to as PEG-Cer).

In some instances, modified forms of lipids may be used including forms modified with detectable labels such as fluorophores. In some instances, the lipid is a lipid analog that emits signal (e.g., a fluorescent signal). Examples include without limitation 1,1′-dioctadecyl-3,3,3′,3′-tetramethylindotricarbocyanine iodide (DiR) and 1,1′-dioctadecyl-3,3,3′,3′-tetramethylindodicarbocyanine (DiD).

Preferably, the lipids are biodegradable in order to allow release of encapsulated agent in vivo and/or in vitro. Biodegradable lipids include but are not limited to 1,2-dioleoyl-sn-glycero-3-phosphocholine (dioleoyl-phosphocholine, DOPC), anionic 1,2-di-(9Z-octadecenoyl)-sn-glycero-3-phospho-(1′-rac-glycerol) (dioleoyl-phosphoglycerol, DOPG), and 1,2-distearoyl-sn-glycero-3-phosphoethanolamine(distearoyl-phosphoethanol-amine, DSPE). Non-lipid membrane components such as cholesterol may also be incorporated.

### Functionalized Lipids or Bilayer Components

At least one component of the lipid bilayer must be functionalized (or reactive). As used herein, a functionalized component is a component that includes a reactive group that can be used to crosslink adjacent bilayers of the multilamellar vesicle. The bilayer component may be modified to include the reactive group.

One or more of the lipids used in the synthesis of the vesicles may be functionalized lipids. As used herein, a functionalized lipid is a lipid having a reactive group that can be used to crosslink adjacent bilayers of the multilamellar vesicle. In some embodiments, the reactive group is one that will react with a crosslinker (or other moiety) to form crosslinks between such functionalized lipids (and thus between lipid bilayers in the vesicle). The reactive group may be located anywhere on the lipid that allows it to contact a crosslinker and be crosslinked to another lipid in an adjacent apposed bilayer. In some embodiments, it is in the head group of the lipid, including for example a phospholipid. An example of a reactive group is a maleimide group. Maleimide groups may be crosslinked to each other in the presence of dithiol crosslinkers such as but not limited to dithiolthrietol (DTT). An example of a functionalized lipid is 1,2-dioleoyl-sn-glycero-3-phosphoethanolamine-N-[4-(p-maleimidop-henyl) butyramide, referred to herein as MPB. Another example of a functionalized lipid is 1,2-distearoyl-sn-glycero-3-phosphoethanolamine-N-[maleimide(polyethylene glycol)2000] (also referred to as maleimide-PEG 2 k-PE). Another example of a functionalized lipid is dioleoyl-phosphatidylethanolamine 4-(N-maleimidomethyl)-cyclohexane-1-carboxylate (DOPE-mal).

It is to be understood that the invention contemplates the use of other functionalized lipids, other functionalized lipid bilayer components, other reactive groups, and other crosslinkers. In addition to the maleimide groups, other examples of reactive groups include but are not limited to other thiol reactive groups, amino groups such as primary and secondary amines, carboxyl groups, hydroxyl groups, aldehyde groups, alkyne groups, azide groups, carbonyls, haloacetyl (e.g., iodoacetyl) groups, imidoester groups, N-hydroxysuccinimide esters, sulfhydryl groups, and pyridyl disulfide groups.

Functionalized and non-functionalized lipids are available from a number of commercial sources including Avanti Polar Lipids (Alabaster, Ala.).

It is to be understood that the invention contemplates various ways to link adjacent bilayers in the multilamellar vesicles to each other. In some instances, crosslinkers are used to effect linkage between adjacent bilayers. The invention however is not so limited.

As an example, vesicles may be formed using click chemistry. An exemplary synthesis method uses alkyne-modified lipids and alkyne-azide chemistry, as follows. Alkyne-modified lipids may be made by mixing the lipids such as 1,2-dioleoyl-sn-glycero-3-phosphoethanolamine (DOPE) with N-hydroxysuccinimide ester of propiolic acid and Et₃N in CDCl₃. The reaction was monitored by NMR. After the reaction is completed, the organic solution may be washed with 5% Na₂CO₃, 1% HCl and brine, dried under Na₂SO₄ and evaporated, and alkyne-modified DOPE weighed. Lipid film with DOPC and alkyne-DOPE in 1:1 molar ratio may be prepared, hydrated, fluidized, and induced to fuse with Mg²⁺ as described previously. MLVs with alkyne-functionalized lipids may be incubated with CuSO₄, copper wire, and 1,14-diazido-3,6,9,12-tetraoxatetradecane for 24 hours at room temperature. Particles may be isolated by centrifugation.

### Crosslinkers

The crosslinker may be a homobifunctional crosslinker or a heterobifunctional crosslinker, depending upon the nature of reactive groups in the lipid bilayers that are being linked to each other. The terms “crosslinker” and “crosslinking agent” are used interchangeably herein. Homobifunctional crosslinkers have two identical reactive groups. Heterobifunctional crosslinkers have two different reactive groups.

In one instance, bilayers (e.g., adjacent bilayers) are crosslinked to each other using the same functionalized lipid (or other bilayer component) and a crosslinker (such as a homobifunctional crosslinker). In another instance, bilayers (e.g., adjacent bilayers) are crosslinked to each other using different functionalized lipids (or other bilayer components) and a crosslinker (such as a heterobifunctional crosslinker).

Various types of commercially available crosslinkers are reactive with one or more of the following groups: maleimides, primary amines, secondary amines, sulfhydryls, carboxyls, carbonyls and carbohydrates. Examples of amine-specific crosslinkers are bis(sulfosuccinimidyl) suberate, bis[2-(succinimidooxycarbonyloxy)ethyl]sulfone, disuccinimidyl suberate, disuccinimidyl tartarate, dimethyl adipimate.2HCl, dimethyl pimelimidate.2HCl, dimethyl suberimidate.2HCl, and ethylene glycolbis-[succinimidyl-[succinate]]. Crosslinkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Crosslinkers preferentially reactive with carbohydrates include azidobenzoyl hydrazine. Crosslinkers preferentially reactive with carboxyl groups include 4-[p-azidosalicylamido]butylamine. Dithiol crosslinkers such as dithiolthietol (DTT), 1,4-di-[3′-(2′-pyridyldithio)-propionamido]butane (DPDPB), and in some instances thiol containing polymers such as (PEG)-SH2 can be used to crosslink maleimide reactive groups.

The structure of DTT is:

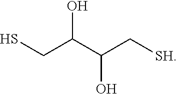

The structure of DPDPB is:

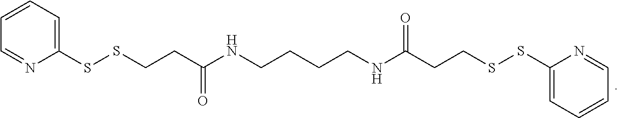 [SMILES: O=C(CCSSc1ccccn1)NCCCCNC(=O)CCSSc1ccccn1]

Crosslinkers reactive with alkyne groups include diazides, such as 1,14-Diazido-3,6,9,12-Tetraoxatetradecane, and other groups compatible with “click” chemistry.

Heterobifunctional crosslinkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[3-dimethylaminopropyl]-carbodiimide hydrochloride. Heterobifunctional crosslinkers that react with carbohydrates and sulfhydryls include 4-[N-maleimidomethyl]-cyclohexane-1-carboxylhydrazide.2HCl, 4-(4-N-maleimidophenyl)-butyric acid hydrazide.2HCl, and 3-[2-pyridyldithio]propionyl hydrazide. Other crosslinkers are bis-[.beta.-4-azidosalicylamido)ethyl]disulfide and glutaraldehyde.

Crosslinkers may be membrane permeable (or lipid soluble) so that they may diffuse through one or more bilayers of the MLVs to effect crosslinking between various layers (e.g., adjacent layers). Any weakly polar/uncharged bifunctional or heterobifunctional small molecule may be an effective membrane permeable crosslinker, particularly if such a molecule includes a reactive group such as but not limited to maleimides, succinimidyl esters, azides, and thiols. Examples of membrane permeable crosslinkers include but are not limited to DTT and 1,4-di-[3′-(2-pyridyldithio)-propionamido]butane (DPDPB).

### Conjugation of Agents

The ICMVs of the invention include at least one molecule of an agent conjugated to a lipid of the vesicle. As used herein, “conjugated” refers to covalent attachment of the agent to the lipid of an ICMV. The agent may be covalently attached to the lipid by reaction of complementary reactive groups on each species. The agent and/or lipid may be functionalized to contain the complementary reactive groups or the reactive groups may be groups already present in the agent or lipid. Examples of pairs of complementary reactive groups include, but are not limited to, thiol and maleimide, amine and carboxylic acid, azide and alkyne, and alkene and tetrazine. Other pairs of complementary reactive groups that may be used to conjugate an agent to a lipid of an ICMV are well-known to one of skill in the art, see, e.g., Hermanson, Bioconjugate Techniques, Academic Press, 3^{rd} ed. 2013 and Sletten et al., Angew Chem Int Ed Engl. 48:6974-98, 2009, which are incorporated herein by reference in their entireties. For example, if the agent is thiol functionalized, the lipid may be maleimide functionalized; if the agent is azide functionalized, the lipid may be alkyne functionalized; if the agent is amine functionalized, the lipid may be carboxylic acid functionalized; or if the agent is alkene functionalized, the lipid may be tetrazine functionalized. For example, the agent may include or be functionalized to include a thiol group and a covalent linkage formed by reaction with lipid functionalized to include a maleimide group. Alternatively, the reactive group on the agent may be an amine or carboxylic acid and the covalent attachment to the lipid could be an amide bond formed by reaction with an amine or carboxylic acid of the lipid. The agent may be conjugated to the lipid prior to ICMV synthesis, and therefore may be encapsulated within the vesicle, between the lipid bilayers of the vesicle, or present on the outer surface of the vesicle. The conjugation of agents does not include conjugation including a cysteine that is within 10 (including within 9, 8, 7, 6, 5, 4, 3, or 2) amino acid residues of or at the amino and/or carboxy terminus of the agent (e.g., an antigen).

Reactive groups to be used to conjugate the agent to the lipid may be the same as those used to crosslink the bilayers, in which case no additional functionalized lipids (or other functionalized components) are required. As an example, if the ICMVs include maleimide functionalized lipids, then the functionalized agent may be thiol-functionalized agent. Alternatively, the reactive groups used to stabilize the vesicles may be different from those used to conjugate the agent to the lipid. Those of ordinary skill in the art will appreciate that other modified versions of agents may be used depending on the nature of the reactive group in the functionalized lipid (or component) in the lipid bilayer of the vesicles. Suitable reactive groups include without limitation amino groups such as primary and secondary amines, carboxyl groups, sulfhydryl groups, hydroxyl groups, aldehyde groups, azide groups, carbonyls, maleimide groups, haloacetyl (e.g., iodoacetyl) groups, imidoester groups, N-hydroxysuccinimide esters, and pyridyl disulfide groups.

### Functionalized Agents

At least one component of the agent must be functionalized or reactive. In some embodiments, a functionalized agent is an agent that includes a reactive group that can be used to conjugate the agent to a lipid (e.g., a functionalized lipid). The agent may be modified to include the reactive group. In some embodiments, the agent is not modified to include the reactive group (e.g., the unmodified agent contains a reactive amine and/or cysteine residue). In some embodiments, a functionalized agent is not a terminal-cysteine-bearing antigen as defined in U.S. Patent Publication No. US2012/0177724, e.g., at p. 8, ¶ [0091], lines 8-30. In some embodiments, a functionalized agent (e.g., an antigen) includes an internal cysteine, which is a cysteine located at the internal region of the functionalized agent (e.g., an antigen). The internal region of a functionalized agent (e.g., an antigen) does not include the 10 amino acids of the amino terminus (i.e., at amino acid positions 1 through 10) and the 10 amino acids of the carboxy terminus ((i.e., at amino acids (n-10) through n, where n represents the number of amino acid residues in the antigen). The internal region of a functionalized agent (e.g., an antigen) includes amino acid 11 to amino acid n-11, wherein n represents the number of amino acid residues in the antigen. In some embodiments, a functionalized agent is a HPV peptide (e.g., a mutant HPV peptide) as described further herein. In some embodiments, a functionalized agent is an adjuvant as described further herein.

One or more of the agents used in the synthesis of the compositions of the invention may be functionalized agents. In some embodiments, the reactive group is one that will react to form a covalent attachment to a lipid. The reactive group may be located anywhere on the agent that allows it to be conjugated to a lipid. An example of a reactive group is a thiol group. It is to be understood that the invention contemplates the use of other functionalized agents and other reactive groups. In addition to the thiol group, other examples of reactive groups include but are not limited to other thiol reactive groups, maleimide groups, hydrazine groups, amino groups such as primary and secondary amines, carboxyl groups, hydroxyl groups, aldehyde groups, alkyne groups, azide groups, alkene groups, tetrazine groups, carbonyls, haloacetyl (e.g., iodoacetyl) groups, imidoester groups, N-hydroxysuccinimide esters, sulfhydryl groups, and pyridyl disulfide groups.

An average of 1-2 molecules of added reactive group (e.g., a thiol) per agent molecule is desirable for efficient conjugation of the agent to the lipid. However, for some agents, addition of more reactive groups (e.g., a thiol molecule) per agent molecule may result in increased conjugation. As such, addition of 2, 3, 4, 5, or more reactive groups (e.g., thiols) per agent molecule is encompassed by the invention.

As an example, agents may be conjugated to lipids by reacting a thiol-functionalized agent with a maleimide functionalized lipid. In some embodiments, a thiol-functionalized agent is not a terminal-cysteine-bearing antigen as defined in U.S. Patent Publication No. US2012/0177724, e.g., at p. 8, [0091], lines 8-30. Thiol-functionalized agents may be prepared using methods known in the art, e.g., 2-iminothiolane-HCl (Traut's reagent), N-succinimidyl S-acetylthioacetate hydrochloride (SATA), or N-Succinimidyl S-acetyl(thiotetraethylene glycol). For example, treatment of an agent containing a primary amine with 10, 20, 30, 40, 50, 60, 70, 80, or more molar equivalents of Traut's reagent at room temperature (e.g., for 1 hour) provides thiol-functionalized agents. An average of 1-2 molecules of added thiol per agent molecule is desirable for efficient conjugation. However, for some agents, addition of more thiol molecules per agent molecule may result in increased conjugation. As such, addition of 2, 3, 4, 5, or more thiol molecule per agent molecule is encompassed by the invention.

In other embodiments, a functionalized agent is an agent that includes a reactive group that can be used to conjugate the agent to a lipophilic moiety (e.g., an acyl hydrocarbon, a fatty alcohol or ester thereof functionalized lipid). A lipophilic moiety refers to a molecule that contains substantially a linear or branched chain of hydrocarbons (e.g., from about 5 to about 50 carbon atoms). In some embodiments, a lipophilic moiety may contain one or more heteroatoms, e.g., 0, N, and/or S. In some embodiments, a lipophilic moiety may contain one or more oxygen atoms. For example, in some embodiments, a lipophilic moiety may contain one or more acyl groups. In some embodiments, a lipophilic moiety may contain one or more alcohol or ester groups. In other embodiments, a lipophilic moiety may contain one or more PEG units interspersed in the chain of hydrocarbons. The function of the lipophilic moiety is to help the agent to insert or embed itself into a lipid bilayer of the ICMV by interacting, e.g., through favorable hydrophobic interactions, with the lipids in the lipid bilayer. Once the agent is functionalized with a lipophilic moiety, the agent can insert itself into a lipid bilayer of the ICMV using the lipophilic moiety. The lipophilic moiety serves as a hydrophobic anchor. In some embodiments, the agent functionalized with a lipophilic moiety is inserted or embedded into a lipid bilayer of the ICMV. In some embodiments, the agent functionalized with a lipophilic moiety is inserted or embedded into a lipid bilayer of the ICMV and is not conjugated to a lipid of the ICMV.

In order to conjugate the agent to the lipophilic moiety, the agent and/or lipophilic moiety may be functionalized to contain the complementary reactive groups or the reactive groups may be groups already present in the agent or the lipophilic moiety. Examples of pairs of complementary reactive groups include, but are not limited to, thiol and maleimide, amine and carboxylic acid, azide and alkyne, and alkene and tetrazine. Other pairs of complementary reactive groups that may be used to conjugate an agent to a lipophilic moiety are well-known to one of skill in the art, see, e.g., Hermanson, Bioconjugate Techniques, Academic Press, 3^{rd} ed. 2013 and Sletten et al., Angew Chem Int Ed Engl. 48:6974-98, 2009, which are incorporated herein by reference in their entireties. For example, if the agent is thiol functionalized, the lipophilic moiety may be maleimide functionalized; if the agent is azide functionalized, the lipophilic moiety may be alkyne functionalized; if the agent is amine functionalized, the lipophilic moiety may be carboxylic acid functionalized; or if the agent is alkene functionalized, the lipophilic moiety may be tetrazine functionalized. In other embodiments, heteroatoms or groups containing heteroatoms (e.g., an acyl group) already present in the lipophilic moiety may be used to conjugate the lipophilic moiety to the functionalized agent.

### PEGylation

The ICMVs may be further modified. The ICMV may be conjugated to polyethylene glycol (PEG) on their surface. PEGylation is used clinically to increase the half-life of various agents including STEALTH liposomes. PEGylation may be accomplished by reacting functionalized lipids on the surface of the stabilized MLVs with a complementary functionalized PEG. The lipids are preferably not conjugated to PEG prior to ICMV synthesis, and rather PEG is conjugated to the ICMV external surface post-synthesis or PEG-lipid conjugates are introduced into the external membrane layer of the particles by “post-insertion” processes.

Reactive groups to be used to PEGylate the ICMVs may be the same as those used to crosslink the bilayers, in which case no additional functionalized lipids (or other functionalized components) are required. As an example, if the ICMVs include maleimide functionalized lipids, then the functionalized PEG may be thiol-PEG. Alternatively, the reactive groups used to stabilize the vesicles may be different from those used to conjugate PEG to the external surface. Those of ordinary skill in the art will appreciate that other modified versions of PEG may be used depending on the nature of the reactive group in the functionalized lipid (or component) in the lipid bilayer of the vesicles. Suitable reactive groups include without limitation amino groups such as primary and secondary amines, carboxyl groups, sulfhydryl groups, hydroxyl groups, aldehyde groups, azide groups, carbonyls, maleimide groups, haloacetyl (e.g., iodoacetyl) groups, imidoester groups, N-hydroxysuccinimide esters, and pyridyl disulfide groups.

Various tests may be performed on the resultant stabilized vesicles in order to determine, inter alia, size and surface charge (e.g., by dynamic light scattering), fraction of lipids on their external surface (e.g., by lamellarity assay), amount of agent incorporated therein or therethrough (e.g., by HPLC). Other tests that may be performed on the vesicles include confocal microscopy and cryo-tunneling electron microscopy (TEM).

### Agents

The invention contemplates the delivery, including in some instances sustained delivery, of agents to regions, tissues or cells in vivo or in vitro using the stabilized lipid vesicles, including the ICMV, of the invention. As used herein, an agent is any atom or molecule or compound that can be used to provide benefit to a subject (including without limitation prophylactic or therapeutic benefit) or that can be used for diagnosis and/or detection (for example, imaging) in vivo or that has use in in vitro applications.

Any agent may be delivered using the compositions (e.g., the stabilized MLVs such as the ICMVs, and compositions thereof including pharmaceutical compositions thereof) and methods of the invention provided that it can be encapsulated into (including throughout) or otherwise carried (e.g., conjugated) on the stabilized MLVs such as the ICMVs provided herein. For example, the agent must be able to withstand the synthesis and optionally storage process for these vesicles. The vesicles may be synthesized and stored in, for example, a lyophilized form, preferably with a sucrose based excipient. The agents, if incorporated into the vesicles during synthesis, should be stable during such storage procedures and times.

The agent may be without limitation a protein, a polypeptide, a peptide, a nucleic acid, a small molecule (e.g., chemical, whether organic or inorganic) drug, a virus-like particle, a steroid, a proteoglycan, a lipid, a carbohydrate, and analogs, derivatives, mixtures, fusions, combinations or conjugates thereof. The agent may be a prodrug that is metabolized and thus converted in vivo to its active (and/or stable) form.

The agents may be naturally occurring or non-naturally occurring. Naturally occurring agents include those capable of being synthesized by the subjects to whom the vesicles are administered. Non-naturally occurring are those that do not exist in nature normally, whether produced by plant, animal, microbe, or other living organism.

One class of agents is peptide-based agents such as (single or multi-chain) proteins and peptides. Examples include antibodies, single chain antibodies, antibody fragments, enzymes, co-factors, receptors, ligands, transcription factors and other regulatory factors, some antigens (as discussed below), cytokines, and chemokines. These peptide-based agents may or may not be naturally occurring but they are capable of being synthesized within the subject, for example, through the use of genetically engineered cells.

Another class of agents that can be delivered using the vesicles of the invention includes those agents that are not peptide-based. Examples include chemical compounds that are non-naturally occurring, or chemical compounds that are not naturally synthesized by mammalian (and in particular human) cells.

A variety of agents that are currently used for therapeutic or diagnostic purposes can be delivered according to the invention and these include without limitation imaging agents, immunomodulatory agents such as immunostimulatory agents and immunoinhibitory agents, antigens, adjuvants, cytokines, chemokines, anti-cancer agents, anti-infective agents, nucleic acids, antibodies or fragments thereof, fusion proteins such as cytokine-antibody fusion proteins, and Fc-fusion proteins.

In some embodiments of the present invention, an agent is not a terminal-cysteine-bearing antigen as defined in U.S. Patent Publication No. US2012/0177724, e.g., at p. 8, ¶ [0091], lines 8-30.

### Imaging Agents

As used herein, an imaging agent is an agent that emits signal directly or indirectly thereby allowing its detection in vivo. Imaging agents such as contrast agents and radioactive agents that can be detected using medical imaging techniques such as nuclear medicine scans and magnetic resonance imaging (MRI). Imaging agents for magnetic resonance imaging (MRI) include Gd(DOTA), iron oxide or gold nanoparticles; imaging agents for nuclear medicine include ²⁰¹Tl, gamma-emitting radionuclide ^{99m}Tc; imaging agents for positron-emission tomography (PET) include positron-emitting isotopes, (18)F-fluorodeoxyglucose ((18)FDG), (18)F-fluoride, copper-64, gadoamide, and radioisotopes of Pb(II) such as 203 Pb, and 11 In; imaging agents for in vivo fluorescence imaging such as fluorescent dyes or dye-conjugated nanoparticles. In other embodiments, the agent to be delivered is conjugated, or fused to, or mixed or combined with an imaging agent.

Immunomodulators

As used herein, an immunomodulator is an agent that stimulates (i.e., an immunostimulator) or inhibits (i.e., an immunoinhibitor) an immune response in a subject to whom it is administered, whether alone or in combination with another agent. In some embodiments, immunomodulators as described herein specifically exclude CureTech's anti-PD-1 antibody CT-011 as described in Patent and Patent Application Publication Nos.: U.S. Pat. No. 8,686,119, WO 2013014668, and WO 2009101611.

Examples of immunomodulators include, but are not limited to, an anti-CTLA-4 antibody, an anti-CD40 antibody, a cyclophosphamide (CPM), an AMD3100, an anti-LAG-3/CD223 antibody, an anti-B7-H5 antibody, an anti-OX40 antibody, an anti-CD28 antibody, an anti-GITR antibody, an anti-4-1BB/CD137 antibody, a 4-1 BB ligand, an anti-BTLA antibody, an anti-TIM-3/HAVCR2 antibody, an anti-KIR antibody, an anti-Flt3/CD135 antibody, an anti-FasL antibody, an anti-CD25 antibody, an GM-CSF, an anti-GM-CSF-receptor (R) antibody, an IL-2, an anti-IL-2-R antibody, an IL-7, an anti-IL-7-R antibody, an IL-21, an anti-IL-21-R antibody, an IL-12, an anti-IL-12-R antibody, an IL-15, an anti-IL-15-R antibody, an IL-18, an anti-IL-18-R antibody, an anti-IDO antibody, an ipilimumab, a crizotinib, a ceritinib, a celecoxib, a SOCS-1 inhibitor, a heat shock protein (HSP), a HSP inhibitor, and an anti-galectin-1 antibody. In some embodiments, one or more immunomodulators are selected from the group consisting of an anti-CTLA-4 antibody, an anti-CD40 antibody, a cyclophosphamide (CPM), and an AMD3100.

Immunostimulators: As used herein, an immunostimulatory agent is an agent that stimulates an immune response (including enhancing a pre-existing immune response) in a subject to whom it is administered, whether alone or in combination with another agent. Examples include antigens, adjuvants (e.g., TLR ligands such as imiquimod and resiquimod, imidazoquinolines, nucleic acids including an unmethylated CpG dinucleotide, monophosphoryl lipid A (MPLA) or other lipopolysaccharide derivatives, single-stranded or double-stranded RNA, flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), AMD3100, immunostimulatory antibodies (e.g., anti-CD40 antibody, anti-CTLA-4 antibody, anti-CD28 antibody, anti-CD3 antibody, or single chain/antibody fragments of these molecules), and PD-1 inhibitors.

The term “PD-1 inhibitor” refers to any agent that inhibits the molecular pathway of PD-1. For example, a PD-1 inhibitor can be an antibody that binds to the PD-1 to block ligand binding to PD-1 (e.g., an anti-PD-1 antibody, nivolumab, and pembrolizumab). A PD-1 inhibitor can also be an antibody that binds to PD-L1 or PD-L2, each of which is a ligand of PD-1, to prevent it from binding to PD-1 (e.g., an anti-PD-L1 antibody (e.g., BMS-936559 and MPDL3280A) and an anti-PD-L2 antibody (see, e.g., Patent Application Publication No.: WO 2010036959 (see, e.g., p. 79, ¶ [0253] through p. 101, ¶ [0296]))).

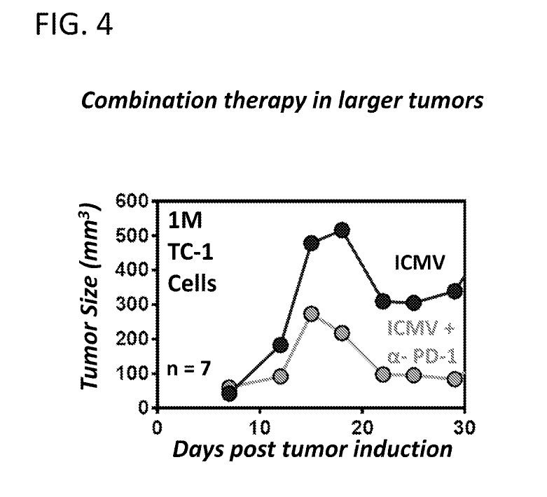

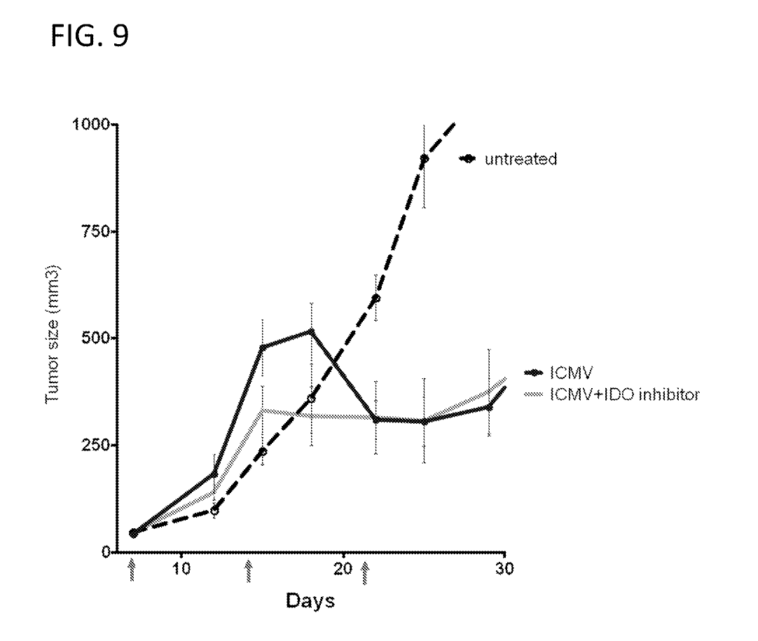

Examples of anti-PD-1 antibodies include nivolumab, pembrolizumab, as well as antibodies described in the following Patent and Patent Application Publication Nos.: WO 2013173223 (see, e.g., p. 3, lines 19-21, p. 8, line 25 through p. 9, line 30, p. 40, line 9 through p. 50, line 24), U.S. Pat. No. 8,008,449 (see, e.g., column 69, Table 2, column 63, line 55 through column 86, line 15), U.S. Pat. No. 8,552,154 (see, e.g., column 67, Table 4, column 57, line 35 through column 74, line 43), U.S. Pat. No. 8,735,553 (see, e.g., column 35, Table 22, column 31, Table 20, column 13, line 9 through column 36, line 62), WO 2004056875 (see, e.g., p. 37, Table 6, p. 33, ¶ [0106] through p. 43, ¶ [0137]), U.S. Pat. No. 7,488,802 (see, e.g., column 22, Tables 6 and 7, column 19, line 62 through column 25, line 4), US 20140294852 (see, e.g., p. 37, Table 2, p. 34, ¶ [0525] through p. 40, ¶ [0585]), U.S. Pat. No. 8,779,105 (see, e.g., column 69, Table 2, column 63, line 55 through column 86, line 11), U.S. Pat. No. 8,741,295 (see, e.g., column 18, line 1 through column 24, line 60), EP 2535354 (see, e.g., p. 23, Table IV, p. 23, line 1 through p. 28, line 35), U.S. Pat. No. 8,168,757 (see, e.g., column 34, line 36 through column 48, line 2), US 20130095098 (see, e.g., p. 12, ¶ [0165] through p. 14, ¶ [0188]), WO 2010029435 (see, e.g., p. 14, Example 1, p. 17, Example 2, pp. 26-28), WO 2014100079 (see, e.g., p. 39, ¶ [00155] through p. 45, ¶ [00174]), U.S. Pat. No. 7,943,743 (see, e.g., column 67, Tables 2 and 3, column 61, line 7 through column 74, line 51), EP 2170959 (see, e.g., p. 26, Table V, p. 16, line 39 through p. 28, line 2), WO 2008156712 (see, e.g., p. 56, Table V, pp. 40-57), and U.S. Pat. No. 8,217,149 (see, e.g., column 99, line 6 through column 118, line 15), each of which is incorporated herein by reference in its entirety. In certain embodiments, the anti-PD-1 antibody has the sequence of nivolumab (see, e.g., FIGS. 4a, 4b, and 9 of U.S. Pat. No. 8,008,449) or pembrolizumab (see, e.g., Patent Application Publication No.: US 2011/0008369). Anti-PD-1 antibodies as described herein specifically exclude CureTech's anti-PD-1 antibody CT-011 as described in Patent and Patent Publication Nos.: U.S. Pat. No. 8,686,119, WO 2013014668, and WO 2009101611.

Examples of anti-PD-L1 antibodies include BMS-936559, MPDL3280A, as well as antibodies described in the following Patent and Patent Application Publication Nos.: U.S. Pat. No. 8,552,154 (see, e.g., column 57, line 35 through column 69, line 16), WO 2014055897 (see, e.g., p. 50, ¶ [00190] through p. 58, ¶ [00219]), WO 2013079174 (see, e.g., p. 48, line 15 through p. 68, line 31), U.S. Pat. No. 8,217,149 (see, e.g., column 99, line 5 through column 118, line 15), U.S. Pat. No. 7,943,743 (see, e.g., column 61, line 9 through column 76, line 45), WO 2014100079 (see, e.g., p. 39, ¶ [00155] through p. 45, ¶ [00175]), U.S. Pat. No. 8,552,154 (see, e.g., column 57, line 35 through column 75, line 13), and U.S. Pat. No. 8,741,295 (see, e.g., column 18, line 32 through column 24, line 60), each of which is incorporated herein by reference in its entirety.

Examples of anti-CD40 antibodies include antibodies described in the following Patent and Patent Application Publication Nos.: US 20030059427 (see, e.g., p. 15, ¶ [0157] through p. 20, ¶ [0212]), WO 2013034904 (see, e.g., p. 58, line 4 through p. 102, line 20), WO 2003029296 (see, e.g., p. 30, line 20 through p. 34, line 16), U.S. Pat. No. 8,637,032 (see, e.g., column 252, line 55 through column 254, line 37), WO 2002028905 (see, e.g., p. 20, line 18 through p. 32, line 30), U.S. Pat. No. 8,778,345 (see, e.g., column 48, line 31 through column 54, line 38), WO 1997031025 (see, e.g., p. 14, line 6 through p. 31, line 26), WO 2012125569 (see, e.g., p. 25, line 33 through p. 27, line 14), WO 2011123489 (see, e.g., p. 93, ¶ [00339] through p. 109, ¶ [00145]), CA 2544949 (see, e.g., p. 78, line 26 through p. 122, line 21), WO 2014070934 (see, e.g., p. 86, line 4 through p. 103, line 4), US 20140093497 (see, e.g., p. 12, ¶ [0112] through p. 13, ¶ [0118]), WO 2010104761 (see, e.g., p. 37, line 3 through p. 66, line 29), U.S. Pat. No. 8,591,900 (see, e.g., column 60, line 14 through column 80, line 29), WO 2007124299 (see, e.g., 68, line 29 through p. p. 88, line 17), U.S. Pat. No. 7,445,780 (see, e.g., column 22, line 29 through column 36, line 39), WO 2006073443 (see, e.g., p. 82, line 6 through p. 89, line 12), WO 2005044294 (see, e.g., p. 137, line 19 through p. 158, line 15), U.S. Pat. No. 5,677,165 (see, e.g., column 11, line 45 through column 18, line 6), WO 2001083755 (see, e.g., p. 39, line 4 through p. 47, line 2), US 20080057070 (see, e.g., p. 26, ¶ [0176] through p. 47, ¶ [0296]), U.S. Pat. No. 7,172,759 (see, e.g., column 9, line 5 through column 12, line 58), WO 2006128103 (see, e.g., p. 75, ¶ [00244] through p. 84, ¶ [000255]), WO 2001016180 (see, e.g., p. 79, line 21 through p. 89, line 14), WO 2003040170 (see, e.g., p. 76, ¶ [0248] through p. 141, ¶ [0239]), U.S. Pat. No. 6,312,693 (see, e.g., column 8, line 51 through column 34, line 45), U.S. Pat. No. 8,492,531 (see, e.g., column 47, line 46 through column 58, line 31), U.S. Pat. No. 8,551,485 (see, e.g., column 78, line 15 through column 85, line 7), U.S. Pat. No. 6,838,261 (see, e.g., column 26, line 10 through column 34, line 26), EP 2243492 (see, e.g., p. 26, ¶ through p. 37, ¶ [0219]), and EP 2011802 (see, e.g., p. 12, ¶ [0047] through p. 40, ¶ [0127]), each of which is incorporated herein by reference in its entirety.

Examples of anti-CD40 antibodies include antibodies described in the following Patent and Patent Application Publication Nos.: US 20030059427 (see, e.g., p. 15, ¶ [0157] through p. 20, ¶ [0212]), WO 2013034904 (see, e.g., p. 58, line 4 through p. 102, line 20), WO 2003029296 (see, e.g., p. 30, line 20 through p. 34, line 16), U.S. Pat. No. 8,637,032 (see, e.g., column 252, line 55 through column 254, line 37), WO 2002028905 (see, e.g., p. 20, line 18 through p. 32, line 30), U.S. Pat. No. 8,778,345 (see, e.g., column 48, line 31 through column 54, line 38), WO 1997031025 (see, e.g., p. 14, line 6 through p. 31, line 26), WO 2012125569 (see, e.g., p. 25, line 33 through p. 27, line 14), WO 2011123489 (see, e.g., p. 93, ¶ [00339] through p. 109, ¶ [00145]), CA 2544949 (see, e.g., p. 78, line 26 through p. 122, line 21), WO 2014070934 (see, e.g., p. 86, line 4 through p. 103, line 4), US 20140093497 (see, e.g., p. 12, ¶ [0112] through p. 13, ¶ [0118]), WO 2010104761 (see, e.g., p. 37, line 3 through p. 66, line 29), U.S. Pat. No. 8,591,900 (see, e.g., column 60, line 14 through column 80, line 29), WO 2007124299 (see, e.g., 68, line 29 through p. p. 88, line 17), U.S. Pat. No. 7,445,780 (see, e.g., column 22, line 29 through column 36, line 39), WO 2006073443 (see, e.g., p. 82, line 6 through p. 89, line 12), WO 2005044294 (see, e.g., p. 137, line 19 through p. 158, line 15), U.S. Pat. No. 5,677,165 (see, e.g., column 11, line 45 through column 18, line 6), WO 2001083755 (see, e.g., p. 39, line 4 through p. 47, line 2), US 20080057070 (see, e.g., p. 26, ¶ [0176] through p. 47, ¶ [0296]), U.S. Pat. No. 7,172,759 (see, e.g., column 9, line 5 through column 12, line 58), WO 2006128103 (see, e.g., p. 75, ¶ [00244] through p. 84, ¶ [000255]), WO 2001016180 (see, e.g., p. 79, line 21 through p. 89, line 14), WO 2003040170 (see, e.g., p. 76, ¶ [0248] through p. 141, ¶ [0239]), U.S. Pat. No. 6,312,693 (see, e.g., column 8, line 51 through column 34, line 45), U.S. Pat. No. 8,492,531 (see, e.g., column 47, line 46 through column 58, line 31), U.S. Pat. No. 8,551,485 (see, e.g., column 78, line 15 through column 85, line 7), U.S. Pat. No. 6,838,261 (see, e.g., column 26, line 10 through column 34, line 26), EP 2243492 (see, e.g., p. 26, through p. 37, ¶ [0219]), and EP 2011802 (see, e.g., p. 12, ¶ [0047] through p. 40, ¶ [0127]), each of which is incorporated herein by reference in its entirety.

Examples of anti-CTLA-4 antibodies include ipilimumab (see, e.g., U.S. Pat. No. 6,682,736 (see, e.g., column 34, line 40 through column 48, line 6)) and antibodies described in the following Patent and Patent Application Publication Nos.: WO 2012120125 (see, e.g., p. 13, line 1 through p. 27, line 18), U.S. Pat. No. 8,017,114 (see, e.g., column 46, line 40 through column 74, line 12), WO 2001014424 (see, e.g., p. 65, line 21 through p. 96, line 15), and WO 2000037504 (see, e.g., p. 56, line 25 through p. 86, line 31), each of which is incorporated herein by reference in its entirety.

Immunoinhibitors:

As used herein, an immunoinhibitory agent is an agent that inhibits an immune response in a subject to whom it is administered, whether alone or in combination with another agent. Examples include steroids, retinoic acid, dexamethasone, cyclophosphamide, anti-CD3 antibody or antibody fragment, and other immunosuppressants. Examples include immunoinhibitory antibodies (e.g., anti-CD3 antibody, or single chain/antibody fragments of this molecule), steroids, retinoic acid, dexamethasone, cyclophosphamide (CPM) (such as those described in the Patent Nos.: U.S. Pat. No. 4,537,883 (see, e.g., column 9, line 62 through column 13, line 6), U.S. Pat. No. 3,808,297 (see, e.g., column 7, line 5 through column 9, line 75), and U.S. Pat. No. 5,036,060 (see, e.g., column 5, line 60 through column 14, line 19), each of which is incorporate herein by reference in its entirety), and other immunosuppressants.

Other immunomodulators include cell-surface makers and antibodies that target cell-surface makers. Examples of immunomodulators such as cell-surface makers and antibodies that target cell-surface makers include anti-LAG-3/CD223 antibodies (such as C9B7W (UniProt ID No. P18627) and those described in the Patent and Patent Application Publication Nos.: WO 2010019570 (see, p. 73, line 4 through e.g., p. 97, line 10), WO 2014008218 (see, e.g., p. 57, line 20 through p. 65, line 17), and WO 2008132601 (see, e.g., p. 15, line 13 through p. 28, line 17)), anti-VISTA/PD-L3 antibodies (such as those described in the Patent and Patent Application Publication Nos.: US 20140105912 (see, e.g., p. 71, through p. 87, ¶ [0755]), U.S. Pat. No. 8,236,304 (see, e.g., column 17, line 7 through column 18, line 48), and US 20110027278 (see, e.g., p. 39, ¶ [0302] through p. 43, ¶ [0333])), anti-B7-H5 antibodies (such as those described in the Patent Application Publication No.: US 20080248007 (see, e.g., p. 9, ¶ [0087] through p. 10, ¶ [0094])), anti-OX40 antibodies (such as those described in the Patent Application Publication No.: WO 201 31 301 02 (see, e.g., p. 31, ¶ [0101] through p. 41, ¶ [0124])), anti-CD28 antibodies (such as those described in the Patent Application Publication No.: EP0440373 (see, e.g., p. 4, line 45 through p. 8, line 37)), anti-GITR antibodies (such as those described in the Patent and Patent Application Publication Nos.: WO 2007133822 (see, e.g., p. 48, line 16 through p. 52, line 18), WO 2009009116 (see, e.g., p. 52, line 30 through p. 56, line 6), WO 2004107618 (see, e.g., p. 78, ¶ [0199] through p. 105, ¶ [0261]), WO 2006105021 (see, e.g., p. 70, line 21 through p. 80, line 31), U.S. Pat. No. 7,812,135 (see, e.g., column 55, line 52 through column 66, line 38), and U.S. Pat. No. 8,591,886 (see, e.g., column 41, line 15 through column 44, line 20)), anti-4-1 BB/CD137 antibodies (such as those described in the U.S. Pat. No. 8,716,452 (see, e.g., column 13, line 55 through column 20, line 62)), 4-1 BB ligands (such as those described in the Patent Application Publication Nos.: WO 1994026290 (see, e.g., 21, line 23 through p. 32, line 33), US 20060110802 (see, e.g., p. 9, ¶ [0098] through p. 16, ¶ [0167]), WO 1999036093 (see, e.g., p. 18, line 5 through p. 56, line 17), WO 2010132389 (see, e.g., p. 30, ¶ [00134] through p. 41, ¶ [00166]), WO 2012145183 (see, e.g., p. 43, line 26 through p. 64, line 12), US 20080008716 (see, e.g., p. 3, ¶ [0042] through p. 7, ¶ [0070]), WO 2004010947 (see, e.g., p. 13, line 12 through p. 23, line 19), and US 20070286860 (see, e.g., p. 27, ¶ [0172] through p. 31, ¶ [200])), anti-BTLA antibodies (such as those described in the Patent and Patent Application Publication Nos.: WO 2010106051 (see, e.g., p. 35, line through p. 35, line 8), WO 2008076560 (see, e.g., p. 85, line 2 through p. 97, line 11), U.S. Pat. No. 8,349,320 (see, e.g., column 47, line 62 through column 72, line 26), and U.S. Pat. No. 8,563,694 (see, e.g., column 56, line 25 through column 65, line 45)), anti-TIM-3/HAVCR2 antibodies (such as those described in the Patent and Patent Application Publication Nos.: U.S. Pat. No. 8,841,418 (see, e.g., column 36, line 45 through column 46, line 47), EP 2417984 (see, e.g., p. 19, ¶ [0137] through p. 28, ¶ [0206]), WO 2014022332 (see, e.g., p. 51, ¶ through p. 54, ¶ [00202]), and U.S. Pat. No. 8,697,069 (see, e.g., p. 40, line 26 through p. 50, line 37)), anti-KIR antibodies (such as those described in the Patent and Patent Application Publication Nos.: WO 2014066532 (see, e.g., p. 25, line 30 through p. 57, line 17), EP 2446897 (see, e.g., p. 45, ¶ [0294] through p. 47, ¶ [0309]), WO 2014055648 (see, e.g., pp. 24-49), and US 20140302052 (see, e.g., p. 2, ¶ through p. 4, ¶ [0044])), anti-Flt3/CD135 antibodies (such as those described in the Patent and Patent Application Publication Nos.: U.S. Pat. No. 6,291,661 (see, e.g., column 23, line 36 through column 38, line 23), EP 0754230 (see, e.g., p. 13, ¶ [0099] through p. 20, ¶ [0138]), EP 0992584 (see, e.g., p. 25, line 28 through p. 30, line 45), and WO 2011076922 (see, e.g., p. 95, ¶ [000269] through p. 82, ¶ [000233])), anti-FasL antibodies (such as those described in the Patent and Patent Application Publication Nos.: US 20100266577 (see, e.g., p. 7, ¶ [0089] through p. 10, ¶ [0122]), US 20070142456 (see, e.g., p. 6, ¶ [0054] through p. 8, ¶ [0066]), WO 2011066211 (see, e.g., pp. 38-97), US 20020187534 (see, e.g., p. 5, ¶ [0061] through p. 10, ¶ [0112]), WO 1999036079 (see, e.g., p. 53, line 15 through p. 60, line 19), and WO 1997033617 (see, e.g., p. 18, line 1 through p. 25, line 17)), and anti-CD25 antibodies (such as those described in the Patent and Patent Application Publication Nos.: WO 2006108670 (see, e.g., pp. 4-8), U.S. Pat. No. 8,182,812 (see, e.g., column 43, line 6 through column 54, line 15), and CA 2585776 (see, e.g., p. 31, ¶ through p. 52, ¶ [00163])).

Other immunomodulators include cytokines or antibodies that target cytokine receptors. Examples of immunomodulators such as cytokines and antibodies that target cytokine receptors include GM-CSF (such as those described in the Patent and Patent Application Publication Nos.: WO 2013074489 (see, e.g., p. 52, line 29 through p. 61, line 26), U.S. Pat. No. 5,891,429 (see, e.g., column 12, line 1 through column 28, line 21), and WO 1989010403 (see, e.g., p. 5, line 35 through p. 13, line 21)), anti-GM-CSF-receptor (R) antibodies (such as those described in the Patent and Patent Application Publication Nos.: WO 1994011404 (see, e.g., pp. 9-28), U.S. Pat. No. 8,263,075 (see, e.g., column 29, line 25 through column 49, line 26), and U.S. Pat. No. 5,932,704 (see, e.g., column 2, line 55 through column 12, line 67)), IL-2 (such as those described in the Patent and Patent Application Publication Nos.: WO 2013130102 (see, e.g., p. 31, ¶ [0101] through p. 41, ¶ [0124]), U.S. Pat. No. 8,349,311 (see, e.g., column 21, line 31 through column 31, line 9), WO 2005007121 (see, e.g., p. 30, line 29 through p. 43, line 31), and WO 1991002000 (see, e.g., pp. 2-7)), anti-IL-2-R antibodies (such as those described in the Patent Application Publication No.: WO 1989009622 (see, e.g., p. 18, line 4 through p. 33, line 12)), IL-7 (such as those described in the Patent and Patent Application Publication Nos.: WO 2012031115 (see, e.g., p. 77, ¶ [00240] through p. 102, ¶ [00347]), WO 2013074489 (see, e.g., p. 52, line 30 through p. 61, line 26), U.S. Pat. No. 7,323,549 (see, e.g., column 12, line 14 through column 24, line 37), and U.S. Pat. No. 8,338,575 (see, e.g., column 11, line 60 through column 24, line 18)), anti-IL-7-R antibodies, IL-21 (as those described in the Patent Application Publication No.: WO 2013169693 (see, e.g., p. 44, line 3 through p. 73, line 15)), anti-IL-21-R antibodies, IL-12 (as those described in the U.S. Pat. No. 8,765,462 (see, e.g., column 27, line 15 through column 50, line 16)), anti-IL-12-R antibodies, IL-15, anti-IL-15-R antibodies, IL-18, anti-IL-8-R antibodies, and anti-IDO antibodies.

Yet other immunomodulators include kinase inhibitors such as crizotinib (see, e.g., Patent Application Publication No.: WO 2013017989 (see, e.g., pp. 54-69)) and ceritinib (see, e.g., Patent Application Publication Nos.: WO 2012082972 (see, e.g., p. 11, line 6 through p. 14, line 17) and WO 2008073687 (see, e.g., p. 34, ¶ [0089] through p. 144, ¶ [0151])), COX-2 inhibitors such as celecoxib (such as those described in the Patent and Patent Application Publication Nos.: WO 2000032189 (see, e.g., p. 38, line 17 through p. 61, line 20), U.S. Pat. No. 6,127,545 (see, e.g., column 7, line 30 through column 17, line 67), WO 2002028270 (see, e.g., p. 37, line 27 through p. 45, line 28), U.S. Pat. No. 6,403,630 (see, e.g., column 16, line 29 through column 17, line 7), and U.S. Pat. No. 5,972,986 (see, e.g., column 5, line 9 through column 16, line 44)), SOCS-1 inhibitors (e.g., PI3K or Jak inhibitors), heat shock proteins (HSP) (such as those described in the Patent and Patent Application Publication Nos.: U.S. Pat. No. 7,678,803 (see, e.g., column 79, line 20 through column 158, line 55), U.S. Pat. No. 7,608,635 (see, e.g., column 93, line 55 through column 108, line 40), U.S. Pat. No. 8,318,790 (see, e.g., column 158, line 51 through column 196, line 20), and US 20130184336 (see, e.g., p. 5, ¶ [0075] through p. 6, ¶ [0093])), HSP inhibitors (such as those described in the U.S. Pat. No. 7,776,849 (see, e.g., column 32, line 47 through column 50, line 67)), and anti-galectin-1 antibodies (such as those described in the Patent Application Publication Nos.: WO 2012131079 (see, e.g., pp. 24-35), WO 2014070214 (see, e.g., p. 42, line 15 through p. 53, line 13), and WO 2014043708 (see, e.g., p. 27, ¶ through p. 36, ¶ [00199])).

The disclosures of the aforementioned Patent and Patent Application Publication Nos. are incorporated herein by reference in their entireties.

Adjuvants

Adjuvant refers to one or more substances that cause stimulation of the immune system. In this context, an adjuvant is used to enhance an immune response to one or more antigens (e.g., a HPV peptide (e.g., a mutant HPV peptide)). An adjuvant may be administered to a subject before, in combination with, or after administration of the antigens (e.g., a HPV peptide (e.g., a mutant HPV peptide)). In some embodiments, an adjuvant may be conjugated to a lipid in the ICMV.

The adjuvant may be without limitation lipids (e.g., monophosphoryl lipid A (MPLA)), alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS1312, water-based nanoparticles combined with a soluble immunostimulant, Seppic).

Adjuvants may be toll-like receptor (TLR) ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

Potentiating Agents

A potentiating agent refers to an agent, such as a molecule (e.g., a small, organic molecule), peptide, protein (e.g., an antibody or an antigen), or a drug that can enhance or augment the effectiveness of a therapeutic compound or composition in treating a disease. The type of potentiating agent used together with the therapeutic compound or composition depends on the specific disease being treated. Potentiating agents include, but are not limited to, anticancer agents, immunostimulatory agents, cancer antigens, anti-infective agents, anti-viral agents, and anti-fungal agents. For example, the composition described herein containing a vesicle (e.g., an ICMV) including a HPV peptide (e.g., E6 and/or E7) for the treatment of cancer (e.g., cervical cancer or a solid tumor cancer) may be used in combination with an anticancer agent as the potentiating agent. PD-1 inhibitors are specifically excluded from the definition of the term “potentiating agent.”

Antigens

The antigen may be without limitation a cancer antigen, a self or autoimmune antigen, a microbial antigen, an allergen, or an environmental antigen. The antigen may be peptide, lipid, or carbohydrate in nature, but it is not so limited. In some embodiments, an antigen is not a terminal-cysteine-bearing antigen as defined in U.S. Patent Publication No. US2012/0177724, e.g., at p. 8, ¶ [0091], lines 8-30. In some embodiments, an antigen includes an internal cysteine, which is a cysteine located at the internal region of the antigen. The internal region of an antigen does not include the 10 amino acids of the amino terminus (i.e., at amino acid positions 1 through 10) and the 10 amino acids of the carboxy terminus ((i.e., at amino acids (n-10) through n, where n represents the number of amino acid residues in the antigen). The internal region of an antigen includes amino acid 11 to amino acid n-11, wherein n represents the number of amino acid residues in the antigen. In some embodiments, an antigen is a HPV peptide (e.g., a mutant HPV peptide) as described herein.

HPV Peptide:

As used herein, a HPV peptide refers to any protein (e.g., a full-length protein or a fragment thereof) within the human papilloma virus (HPV) structure or a mutant thereof (e.g., a mutant HPV peptide). A mutant HPV peptide refers to a HPV peptide that has at least one (e.g., two, three, four, five, six, seven, etc., e.g., two to six) amino acid substitutions relative to a wild-type HPV peptide sequence (e.g., a sequence of any one of SEQ ID NOs: 1-4). Proteins that make up the structure of the HPV are E1, E2, E4, E5, E6, E7, L1, and L2. The sequences of each of the structural proteins may be different depending on the specific HPV genotype. More than 150 HPV genotypes have been identified. Twelve HPV genotypes (HPV-16, -18, -31, -33, -35, -39, -45, -51, -52, -56, -58, and -59) are classified as “carcinogenic to humans.” HPV-68 is classified as “probably carcinogenic.” Other HPV genotypes (such as HPV-67, -73, -82, -66, -53, and -70) are classified as “possibly carcinogenic.” In some embodiments, a HPV peptide is derived from a HPV of genotype 16 (HPV-16) or 18 (HPV-18). In some embodiments, a HPV peptide is derived from a HPV E6 protein or a HPV E7 protein. In some embodiments, a HPV peptide derived from a HPV-16 E6 protein (e.g., the HPV peptide is a full-length, wild-type HPV-16 E6 protein or a mutant and/or fragment thereof). In some embodiments, a HPV peptide derived from a HPV-16 E7 protein (e.g., the HPV peptide is a full-length, wild-type HPV-16 E7 protein or a mutant and/or fragment thereof). In some embodiments, a HPV peptide derived from a HPV-18 E6 protein (e.g., the HPV peptide is a full-length, wild-type HPV-18 E6 protein or a mutant and/or fragment thereof). In some embodiments, a HPV peptide derived from a HPV-18 E7 protein (e.g., the HPV peptide is a full-length, wild-type HPV-18 E7 protein or a mutant and/or fragment thereof). Sequences of full-length, wild-type HPV-16 E6, HPV-16 E7, HPV-18 E6, and HPV-18 E7 peptides, and mutants thereof (e.g., mutant HPV peptides) are shown in SEQ ID NOs: 1-9 below.

A fragment of a HPV peptide refers to a peptide that has a length from about 20 amino acids to the full-length of the HPV protein (e.g., a wild-type or mutant HPV peptide having the sequence of any one of SEQ ID NOs: 1-9). For example, a HPV peptide that is a fragment may contain 20 amino acids to 158 amino acids (e.g., 20, 25, 30, 35, 40, 45, 50, 55, 60, 65, 70, 75, 80, 85, 90, 95, 100, 105, 110, 115, 120, 125, 130, 135, 140, 145, 150, 155, or 158 amino acids). In some embodiments, a HPV peptide may be a fragment of a fusion protein of HPV E6 and E7 peptides (e.g., a fragment of the fusion protein having the sequence of SEQ ID NO: 10). A HPV peptide that is a fragment of a fusion protein of HPV E6 and E7 peptides (e.g., a fragment of the fusion protein having the sequence of SEQ ID NO: 10) may contain 20 amino acids to the full-length of the fusion protein (e.g., 256 amino acids in SEQ ID NO: 10) (e.g., 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, or 256 amino acids). In some embodiments, a HPV peptide may have at least 80% (e.g., 80%, 82%, 85%, 87%, 90%, 92%, 95%, 97%, 99%, or 100%) amino acid sequence identity over its full-length to a sequence of a wild-type HPV protein or a fusion protein containing two different HPV proteins. In some embodiments, a HPV peptide may have at least 80% (e.g., 80%, 82%, 85%, 87%, 90%, 92%, 95%, 97%, 99%, or 100%) amino acid sequence identity over its full-length to a sequence of any one of SEQ ID NOs: 1-10.

In some embodiments, a multilamellar lipid vesicle having crosslinks between lipid bilayers includes one or more mutant HPV peptides (e.g., mutant HPV peptides having sequences of SEQ ID NOs: 5-9), wherein at least one of the one or more mutant HPV peptides is conjugated to a lipid of the multilamellar lipid vesicle.

In some embodiments, a multilamellar lipid vesicle having crosslinks between lipid bilayers includes one or more first mutant HPV peptides and one or more second mutant HPV peptides, wherein at least one of the first mutant HPV peptides is conjugated to a first lipid of the multilamellar lipid vesicle, wherein at least one of the second mutant HPV peptides is conjugated to a second lipid of the multilamellar lipid vesicle, and wherein the first and second mutant HPV peptides are different. The first and/or second mutant HPV peptides may have a sequence of any one of SEQ ID NOs: 5-9.

In some embodiments, a multilamellar lipid vesicle having crosslinks between lipid bilayers includes one or more HPV peptides (e.g., a wild-type or mutant HPV peptide having the sequence of any one of SEQ ID NOs: 1-9), wherein at least one molecule of the one or more HPV peptides is conjugated to a lipid of the multilamellar lipid vesicle, and wherein the conjugation does not comprise a cysteine.

In some embodiments, a multilamellar lipid vesicle having crosslinks between lipid bilayers includes one or more HPV peptides (e.g., a wild-type or mutant HPV peptide having the sequence of any one of SEQ ID NOs: 1-9), wherein at least one molecule of the one or more HPV peptides is conjugated to a lipid of the multilamellar lipid vesicle, and wherein the conjugation does not comprise a cysteine that is within 10 (including within 9, 8, 7, 6, 5, 4, 3, or 2) amino acid residues of or at the amino and/or carboxy terminus of the HPV peptide.

In some embodiments, a HPV peptide includes a fusion protein of a HPV E6 peptide and a HPV E7 peptide. In some embodiments, the HPV E6 peptide in the fusion protein is derived from a HPV-16 E6 protein or a HPV-18 E6 protein (e.g., the HPV E6 peptide is a full-length, wild-type HPV-16 E6 or HPV-18 E6 protein or a mutant and/or fragment thereof). In some embodiments, the HPV E7 peptide in the fusion protein is derived from a HPV-16 E7 protein or a HPV-18 E7 protein (e.g., the HPV E7 peptide is a full-length, wild-type HPV-16 E7 or HPV-18 E7 protein or a mutant and/or fragment thereof). An example of the sequence of a fusion protein of a HPV-16 E6 peptide and a HPV-16 E7 peptide is shown in SEQ ID NO: 10 below.

| SEQ ID NO: 1: wild-type, full-length HPV-16 E6 peptide | |
|---|---|
| 1-60 | MHQKRTAMFQ DPQERPRKLP QLCTELQTTI HDIILECVYC KQQLLRREVY DFAFRDLCIV |
| 61-120 | YRDGNPYAVC DKCLKFYSKI SEYRHYCYSL YGTTLEQQYN KPLCDLLIRC INCQKPLCPE |
| 121-158 | EKQRHLDKKQ RFHNIRGRWT GRCMSCCRSS RTRRETQL |
| SEQ ID NO: 2: wild-type, full-length HPV-16 E7 peptide | |
| 1-60 | MHGDTPTLHE YMLDLQPETT DLYCYEQLND SSEEEDEIDG PAGQAEPDRA HYNIVTFCCK |
| 61-98 | CDSTLRLCVO STHVDIRTLE DLLMGTLGIV CPICSOKP |
| SEQ ID NO: 3: wild-type, full-length HPV-18 E6 peptide | |
| 1-60 | LPDLCTELNT SLQDIEITCV YCKTVLELTE VFEFAFKDLF VVYRDSIPHA ACHKCIDFYS |
| 61-120 | RIRELRYYSD SVYGDTLEKL TNTGLYNLLI RCLRCQKPLN PAEKLRHLNE KRRFHKIAGH |
| 121-145 | YRGQCHSCCN RARQERLQRR RETQV |
| SEQ ID NO: 4: wild-type, full-length HPV-18 E7 peptide | |
| 1-60 | MYGPKATLQD IVLHLEPQNE IPVDLLCHEQ LSDSEEENDE IDGVNHQHLP ARRAEPQRHT |
| 61-120 | MLCMCCKCEA RIELVVESSA DDLRAFQQLF LSTLSFVCPW CASQQ |
| SEQ ID NO: 5: mutant, full-length HPV-16 E6 peptide | |
| 1-60 | MHQKRTAMFQ DPQERPRKLP QLCTELQTTI HDIILECVYC KQQLLRREVY DFAFRDLCIV |
| 61-120 | YRDGNPYAVG DKCLKFYSKI SEYRHYCYSL YGTTLEQQYN KPLCDLLIRC INGQKPLCPE |
| 121-158 | EKQRHLDKKQ RFHNGRGRWT GRCMSCCRSS RTRRETQL |
| SEQ ID NO: 6: mutant, full-length HPV-16 E7 peptide | |
| 1-60 | MHGDTPTLHE YMLDLQPETT DLYGYGQLND SSEEEDEIDG PAGQAEPDRA HYNIVTFCCK |
| 61-98 | CDSTLRLCVQ STHVDIRTLE DLLMGTLGIV CPICSQKP |
| SEQ ID NO: 7: mutant, full-length HPV-18 E6 peptide_1 | |
| 1-60 | LPDLCTELNT SLQDIEITCV YCKTVLELTE VFEFAFKDLF VVYRDSIPHA AGHKCIDFYS |
| 61-120 | RIRELRYYSD SVYGDTLEKL TNTGLYNLLI RCLRGQKPLN PAEKLRHLNE KRRFHKGAGH |
| 121-145 | YRGQCHSCCN RARQERLQRR RETQV |
| SEQ ID NO: 8: mutant, full-length HPV-18 E6 peptide_2 | |
| 1-60 | MARFEDPTRR PYKLPDLCTE LNTSLQDIEI TCVYCKTVLE LTEVFEFAFK DLFVVYRDSI |
| 61-120 | PHAAGHKCID FYSRIRELRH YSDSVYGDTL EKLTNTGLYN LLIRCLRGQK PLNPAEKLRH |
| 121-145 | LNEKRRFHNG AGHYRGQCHS CCNRARQERL QRRRETQV |
| SEQ ID NO: 9: mutant, full-length HPV-18 E7 peptide | |
| 1-60 | MHGPKATLQD IVLHLEPQNE IPVDLLGHGQ LSDSEEENDE IDGVNHQHLP ARRAEPQRHT |
| 61-105 | MLCMCCKCEA RIKLVVESSA DDLRAFQQLF LNTLSFVCPW CASQQ |
| SEQ ID NO: 10: fusion protein of HPV-16 E6 peptide (SEQ ID NO: 1) and HPV-16 E7 | |
| peptide | |
| (SEQ ID NO: 2) | |
| 1-60 | MHQKRTAMFQ DPQERPRKLP QLCTELQTTI HDIILECVYC KQQLLRREVY DFAFRDLCIV |
| 61-105 | YRDGNPYAVC DKCLKFYSKI SEYRHYCYSL YGTTLEQQYN KPLCDLLIRC INCQKPLCPE |
| 121-180 | EKQRHLDKKQ RFHNIRGRWT GRCMSCCRSS RTRRETQLMH GDTPTLHEYM LDLQPETTDL |
| 181-240 | YCYEQLNDSS EEEDEIDGPA GQAEPDRAHY NIVTFCCKCD STLRLCVQST HVDIRTLEDL |
| 241-256 | LMGTLGIVCP ICSQKP |

In certain embodiments, a composition of the invention comprises: a multilamellar lipid vesicle having crosslinks between lipid bilayers and including one or more HPV peptides, and a PD-1 inhibitor (e.g., a wild-type or mutant HPV E6 and/or E7 peptide (e.g., any one of SEQ ID NOs: 1-10) and an anti-PD-1 antibody).

In certain embodiments, a composition of the invention comprises: a multilamellar lipid vesicle having crosslinks between lipid bilayers and including one or more HPV peptides (e.g., a wild-type HPV peptide having the sequence of SEQ ID NO: 1 or 2).

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and an anti-CTLA antibody.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and an anti-PD-1 antibody.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and an anti-PD-L1 antibody.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and cyclophosphamide.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and an anti-PD-1 antibody, and cyclophosphamide.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and an indoleamine-2,3-dioxygenase (IDO) inhibitor 1-methyltryptophan.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and monophosphoryl lipid A.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and a potentiating agent.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and a TLR-4 agonist.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and a TLR-7 agonist.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and an anti-CD40 antibody.

In certain embodiments, a composition of the invention comprises: a multilamellar lipid vesicle having crosslinks between lipid bilayers and including one or more HPV peptides having the sequence of SEQ ID NO: 1, and a multilamellar lipid vesicle having crosslinks between lipid bilayers and including one or more HPV peptides having the sequence of SEQ ID NO: 2, and an anti-PD-1 antibody, and AMD3100.

Cancer Antigens:

A cancer antigen is an antigen that is expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, and MAGE-05. The cancer antigen may be selected from the group consisting of GAGE-1, GAGE-2, GAGE-3, GAGE-4, GAGE-5, GAGE-6, GAGE-7, GAGE-8, and GAGE-9. The cancer antigen may be selected from the group consisting of BAGE, RAGE, LAGE-1, NAG, GnT-V, MUM-1, CDK4, tyrosinase, p53, a member of the mucin (MUC) family (e.g., MUC1), HER2/neu, p21 ras, RCAS1, α-fetoprotein, E-cadherin, .α-catenin, β-catenin, γ-catenin, p120ctn, gp100.sup.Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus (HPV) proteins or peptides (e.g., HPV peptides as described above), Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, and c-erbB-2. In some embodiments, a cancer antigen may be associated with a cancerous tumor (i.e., a tumor-associated antigen). A tumor-associated antigen is often associated with a specific type of tumor, such as a lymphoma, a carcinoma, a sarcoma, or a melanoma. A tumor-associated antigen may elicit cellular and/or humoral immune responses against the tumor. An example of a tumor-associated antigen is a HPV peptide as described herein, in particular, a HPV-16 E6 or E7 peptide or a HPV-18 E6 or E7 peptide. In some embodiments, a HPV peptide may be a fusion protein of a HPV E6 and E7 peptide.

In some embodiments, the composition of the invention contains a multilamellar lipid vesicle (e.g., an ICMV) having crosslinks between lipid bilayers and including one or more cancer antigens. In some embodiments, a cancer antigen in a multilamellar lipid vesicle is not a terminal-cysteine-bearing antigen as defined in U.S. Patent Publication No. US2012/0177724, e.g., at p. 8, ¶ [0091], lines 8-30. In certain embodiments, the cancer antigen may be a gp100, a NY-ESO-1, a member of the mucin (MUC) family (e.g., MUC1), or may be selected from a group consisting of a MAGE-A1, a MAGE-A2, a MAGE-A3, a MAGE-A4, a MAGE-A5, a MAGE-A6, a MAGE-A7, a MAGE-A8, a MAGE-A9, a MAGE-A10, a MAGE-A11, a MAGE-A12, a MAGE-Xp2 (MAGE-B2), a MAGE-Xp3 (MAGE-B3), a MAGE-Xp4 (MAGE-B4), a MAGE-C1, a MAGE-C2, a MAGE-C3, a MAGE-C4, and a MAGE-05.

In certain embodiments, the cancer antigen may be Wilms' tumor antigen 1 (WT1), MUC1, LMP2 (latent membrane protein 2 from Epstein-Barr virus), EGFRvIII, Her2/neu, Idiotype antigens (see, e.g., Weng et al., J. Clin Oncol. 22:4717-24, 2004), MAGE-A3, non-mutant p53, NY-ESO-1, PSMA (prostate-specific membrane antigen), GD2, CEA (carcinoembryonic antigen), MelanA/MART1, Ras mutants, gp100, mutant p53, Proteinase3 (PR1), BCR-Abl breakpoints (see, e.g., Maslak et al., Leukemia 22:1613-6, 2008), Tyrosinase, Survivin, PSA (prostate-specific antigen), hTERT (human telomerase), sarcoma translocation breakpoints (see, e.g., Mackall et al., Clin Cancer Res. 14:4850-8, 2008), EphA2, PAP (prostatic acid phosphatase), ML-IAP (ML-inhibitor of apoptosis), AFP (alphafetoprotein), EpCAM (epithelial cell adhesion molecule), ERG (TMPRSS2 ETS fusion gene), NA17, PAX3 (paired box 3), Androgen receptor, Cyclin B1, Polysialic acid, MYCN (N-myc), RhoC, TRP-2 (tyrosinase-related protein 2), GD3, Fucosyl GM1, Mesothelin, PSCA (prostate stem cell antigen), MAGE-A1, CYP1B1 (cytochrome P450 1B1), PLAC1 (placenta-specific 1), GM3, BORIS (brother of the regulator of imprinted sites), Tn (N-acetylgalactosamine linked to serine or threonine by a glycosidic bond), GloboH, ETV6-AML, NY-BR-1, RGS5 (regulator of G protein signaling 5), SART3 (squamous cell carcinoma antigen recognized by T cells 3), STn (sialyl Tn antigen), Carbonic anhydrase IX, PAX5 (paired box 5), OY-TES1, Sperm protein 17, LCK (p56 form), HMWMAA (high molecular weight melanoma associated antigen), AKAP-4 (A-kinase anchor protein 4), SSX2 (synovial sarcoma breakpoint 2), XAGE1 (x antigen 1), B7H3, Legumain, Tie 2, Page4, VEGFR2 (vascular endothelial growth factor receptor 2), MAD-CT-1 (melanoma cancer testis antigen-1), FAP (fibroblast activation protein), PDGFR-b (platelet-derived growth factor receptor-b), MAD-CT-2 (melanoma cancer testis antigen-2), or Fos-related antigen 1.

Microbial Antigens:

Microbial antigens are antigens derived from microbial species such as without limitation bacterial, viral, fungal, parasitic and mycobacterial species. As such, microbial antigens include bacterial antigens, viral antigens, fungal antigens, parasitic antigens, and mycobacterial antigens. Examples of bacterial, viral, fungal, parasitic and mycobacterial species are provided herein. The microbial antigen may be part of a microbial species or it may be the entire microbe.

Allergens

An allergen is an agent that can induce an allergic or asthmatic response in a subject. Allergens include without limitation pollens, insect venoms, animal dander dust, fungal spores and drugs (e.g., penicillin). Examples of natural, animal and plant allergens include but are not limited to proteins specific to the following genera: Canine (Canis familiaris); Dermatophagoides (e.g., Dermatophagoides farinae); Felis (Felis domesticus); Ambrosia (Ambrosia artemiisfolia); Lolium (e.g., Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g., Plantago lanceolata); Parietaria (e.g., Parietaria officinalis or Parietaria judaica); Blattella (e.g., Blattella germanica); Apis (e.g., Apis multiflorum); Cupressus (e.g., Cupressus sempervirens, Cupressus arizonica, and Cupressus macrocarpa); Juniperus (e.g., Juniperus sabinoides, Juniperus virginiana, Juniperus communis, and Juniperus ashei); Thuya (e.g., Thuya orientalis); Chamaecyparis (e.g., Chamaecyparis obtusa); Periplaneta (e.g., Periplaneta americana); Agropyron (e.g., Agropyron repens); Secale (e.g., Secale cereale); Triticum (e.g., Triticum aestivum); Dactylis (e.g., Dactylis glomerata); Festuca (e.g., Festuca elatior); Poa (e.g., Poa pratensis or Poa compressa); Avena (e.g., Avena sativa); Holcus (e.g., Holcus lanatus); Anthoxanthum (e.g., Anthoxanthum odoratum); Arrhenatherum (e.g., Arrhenatherum elatius); Agrostis (e.g., Agrostis alba); Phleum (e.g., Phleum pratense); Phalaris (e.g., Phalaris arundinacea); Paspalum (e.g., Paspalum notatum); Sorghum (e.g., Sorghum halepensis); and Bromus (e.g., Bromus inermis).

Anti-Cancer Agents

As used herein, an anti-cancer agent is an agent that at least partially inhibits the development or progression of a cancer, including inhibiting in whole or in part symptoms associated with the cancer even if only for the short term. Several anti-cancer agents can be categorized as DNA damaging agents and these include topoisomerase inhibitors (e.g., etoposide, ramptothecin, topotecan, teniposide, mitoxantrone), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), anthracyclines, vinca alkaloids, or epipodophyllotoxins.

Examples of anti-cancer agents include without limitation Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Bortezomib (VELCADE); Brequinar Sodium; Bropirimine; Busulfan; Cactinomycin; Calusterone; Caracemide; Carbetimer; Carboplatin (a platinum-containing regimen); Carmustine; Carubicin Hydrochloride; Carzelesin; Cedefingol; Chlorambucil; Cirolemycin; Cisplatin (a platinum-containing regimen); Cladribine; Crisnatol Mesylate; Cyclophosphamide; Cytarabine; Dacarbazine; Dactinomycin; Daunorubicin; Decitabine; Dexormaplatin; Dezaguanine; Diaziquone; Docetaxel (TAXOTERE); Doxorubicin; Droloxifene; Dromostanolone; Duazomycin; Edatrexate; Eflornithine; Elsamitrucin; Enloplatin; Enpromate; Epipropidine; Epirubicin; Erbulozole; Erlotinib (TARCEVA), Esorubicin; Estramustine; Etanidazole; Etoposide; Etoprine; Fadrozole; Fazarabine; Fenretinide; Floxuridine; Fludarabine; 5-Fluorouracil; Fluorocitabine; Fosquidone; Fostriecin; Gefitinib (IRESSA), Gemcitabine; Hydroxyurea; Idarubicin; Ifosfamide; Ilmofosine; Imatinib mesylate (GLEEVAC); Interferon alpha-2a; Interferon alpha-2b; Interferon alpha-n1; Interferon alpha-n3; Interferon beta-I a; Interferon gamma-I b; Iproplatin; Irinotecan; Lanreotide; Lenalidomide (REVLIMID, REVIMID); Letrozole; Leuprolide; Liarozole; Lometerxol; Lomustine; Losoxantrone; Masoprocol; Maytansine; Mechlorethamine; Megestrol; Melengestrol; Melphalan; Menogaril; Mercaptopurine; Methotrexate; Metoprine; Meturedepa; Mitindomide; Mitocarcin; Mitocromin; Mitogillin; Mitomalcin; Mitomycin; Mitosper; Mitotane; Mitoxantrone; Mycophenolic Acid; Nocodazole; Nogalamycin; Ormaplatin; Oxisuran; Paclitaxel; Pemeterxed (ALIMTA), Pegaspargase; Peliomycin; Pentamustine; Pentomone; Peplomycin; Perfosfamide; Pipobroman; Piposulfan; Piritrexim Isethionate; Piroxantrone; Plicamycin; Plomestane; Porfimer; Porfiromycin; Prednimustine; Procarbazine; Puromycin; Pyrazofurin; Riboprine; Rogletimide; Safingol; Semustine; Simtrazene; Sitogluside; Sparfosate; Sparsomycin; Spirogermanium; Spiromustine; Spiroplatin; Streptonigrin; Streptozocin; Sulofenur; Talisomycin; Tamsulosin; Taxol; Taxotere; Tecogalan; Tegafur; Teloxantrone; Temoporfin; Temozolomide (TEMODAR); Teniposide; Teroxirone; Testolactone; Thalidomide (THALOMID) and derivatives thereof; Thiamiprine; Thioguanine; Thiotepa; Tiazofurin; Tirapazamine; Topotecan; Toremifene; Trestolone; Triciribine; Trimeterxate; Triptorelin; Tubulozole; Uracil Mustard; Uredepa; Vapreotide; Verteporfin; Vinblastine; Vincristine; Vindesine; Vinepidine; Vinglycinate; Vinleurosine; Vinorelbine; Vinrosidine; Vinzolidine; Vorozole; Zeniplatin; Zinostatin; Zorubicin.

The anti-cancer agent may be an enzyme inhibitor including without limitation a tyrosine kinase inhibitor, a cyclin-dependent kinase (CDK) inhibitor, a mitogen-activated protein (MAP) kinase inhibitor, or an epidermal growth factor receptor (EGFR) inhibitor. The tyrosine kinase inhibitor may be without limitation Genistein (4′,5,7-trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-1-(3′-carboxy-4′-hydroxyphenyl)-2-(2″,5″-dihydroxy-phenyl)ethane, or HDBA (2-Hydroxy-5-(2,5-Dihydroxybenzylamino)-2-hydroxybenzoic acid. The CDK inhibitor may be without limitation p21, p27, p57, p15, p16, p18, or p19. The MAP kinase inhibitor may be without limitation KY12420 (C.sub.23H.sub.24O.sub.8), CNI-1493, PD98059, or 4-(4-Fluorophenyl)-2-(4-methylsulfinyl phenyl)-5-(4-pyridyl) 1H-imidazole. The EGFR inhibitor may be without limitation erlotinib (TARCEVA), gefitinib (IRESSA), WH1-P97 (quinazoline derivative), LFM-A12 (leflunomide metabolite analog), ABX-EGF, lapatinib, canertinib, ZD-6474 (ZACTIMA), AEE788, and AG1458.

The anti-cancer agent may be a vascular endothelial growth factor (VEGF) inhibitor including without limitation bevacizumab (AVASTIN), ranibizumab (LUCENTIS), pegaptanib (MACUGEN), sorafenib, sunitinib (SUTENT), vatalanib, ZD-6474 (ZACTIMA), anecortave (RETAANE), squalamine lactate, and semaphorin.

The anti-cancer agent may be an antibody or an antibody fragment including without limitation an antibody or an antibody fragment including but not limited to bevacizumab (AVASTIN), trastuzumab (HERCEPTIN), alemtuzumab (CAMPATH, indicated for B cell chronic lymphocytic leukemia,), gemtuzumab (MYLOTARG, hP67.6, anti-CD33, indicated for leukemia such as acute myeloid leukemia), rituximab (RITUXAN), tositumomab (BEXXAR, anti-CD20, indicated for B cell malignancy), MDX-210 (bispecific antibody that binds simultaneously to HER-2/neu oncogene protein product and type I Fc receptors for immunoglobulin G (IgG) (Fc gamma RD), oregovomab (OVAREX, indicated for ovarian cancer), edrecolomab (PANOREX), daclizumab (ZENAPAX), palivizumab (SYNAGIS, indicated for respiratory conditions such as RSV infection), ibritumomab tiuxetan (ZEVALIN, indicated for Non-Hodgkin's lymphoma), cetuximab (ERBITUX), MDX-447, MDX-22, MDX-220 (anti-TAG-72), IOR-05, IOR-T6 (anti-CD1), IOR EGF/R3, celogovab (ONCOSCINT OV103), epratuzumab (LYMPHOCIDE), pemtumomab (THERAGYN), and Gliomab-H (indicated for brain cancer, melanoma).

Hematopoietic Differentiating Agents

The agent may be one that stimulates the differentiation of hematopoietic progenitor cells towards one or more lineages. Examples include without limitation IL-3, G-CSF, GM-CSF, M-CSF, thrombopoietin, erythropoietin, WntSA, and Wnt11A.

Hematopoietic Self-Renewing Agents

The agent may be one that stimulates the self-renewal of hematopoietic progenitor cells. Examples include without limitation kit ligand, GSK3-beta inhibitors, Wnt5A together with SLF, Notch1 activators, Lnk inhibitors, prostaglandin E2 (PGE2) and agents that stimulate the PGE2 pathway including PGE2, PGI2, Linoleic Acid, 13(s)-NODE, LY171883, Mead Acid, Eicosatrienoic Acid, Epoxyeicosatrienoic Acid, ONO-259, Cay1039, a PGE2 receptor agonist, of 16,16-dimethyl PGE2, 19(R)-hydroxy PGE2, 16,16-dimethyl PGE2 p-(p-acetamidobenzamido) phenyl ester, 11-deoxy-16,16-dimethyl PGE2,9-deoxy-9-methylene-16,16-dimethyl PGE2,9-deoxy-9-methylene PGE2, Butaprost, Sulprostone, PGE2 serinol amide, PGE2 methyl ester, 16-phenyl tetranor PGE2,15(S)-15-methyl PGE2,15(R)-15-methyl PGE2, BIO, 8-bromo-cAMP, Forskolin, Bapta-AM, Fendiline, Nicardipine, Nifedipine, Pimozide, Strophanthidin, Lanatoside, L-Arg, Sodium Nitroprusside, Sodium Vanadate, Bradykinin, Mebeverine, Flurandrenolide, Atenolol, Pindolol, Gaboxadol, Kynurenic Acid, Hydralazine, Thiabendazole, Bicuclline, Vesamicol, Peruvoside, Imipramine, Chlorpropamide, 1,5-Pentamethylenetetrazole, 4-Aminopyridine, Diazoxide, Benfotiamine, 12-Methoxydodecenoic acid, N-Formyl-Met-Leu-Phe, Gallamine, IAA 94, Chlorotrianisene, and derivatives thereof.

Anti-Infective Agents

The agent may be an anti-infective agent including without limitation an anti-bacterial agent (e.g., an anti-mycobacterial agent), an anti-viral agent, an anti-parasitic agent, and an anti-fungal agent.

Anti-bacterial agents may be without limitation beta.-lactam antibiotics, penicillins (such as natural penicillins, aminopenicillins, penicillinase-resistant penicillins, carboxy penicillins, ureido penicillins), cephalosporins (first generation, second generation, and third generation cephalosporins), other beta-lactams (such as imipenem, monobactams), beta-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, or quinolines.

Other anti-bacterials may be without limitation Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfas alazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; or Zorbamycin. Anti-mycobacterial agents may be without limitation Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate or Trecator-SC (Ethionamide).

Anti-viral agents may be without limitation amantidine and rimantadine, ribivarin, acyclovir, vidarabine, trifluorothymidine, ganciclovir, zidovudine, retinovir, and interferons. Anti-viral agents may be without limitation further include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime or integrase inhibitors.

Anti-fungal agents may be without limitation imidazoles and triazoles, polyene macrolide antibiotics, griseofulvin, amphotericin B, and flucytosine. Antiparasite agents include heavy metals, antimalarial quinolines, folate antagonists, nitroimidazoles, benzimidazoles, avermectins, praxiquantel, ornithine decarboxylase inhibitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); or sulfated naphthylamine (e.g., suramin). Other anti-infective agents may be without limitation Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (CECLOR); Acyclovir (ZOVIRAX); Norfloxacin (NOROXIN); Cefoxitin (MEFOXIN); Cefuroxime axetil (CEFTIN); Ciprofloxacin (Cipro); Aminacrine Hydrochloride; Benzethonium Chloride: Bithionolate Sodium; Bromchlorenone; Carbamide Peroxide; Cetalkonium Chloride; Cetylpyridinium Chloride: Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal; or Troclosene Potassium.

Nucleic Acid Agents

Nucleic acids that can be delivered to a subject according to the invention include naturally or non-naturally occurring DNA (including cDNA, genomic DNA, nuclear DNA, mitochondrial DNA), RNA (including mRNA, rRNA, tRNA), oligonucleotides, a triple-helix forming molecule, immunostimulatory nucleic acids such as those described in U.S. Pat. No. 6,194,388 (the teachings of which relating to immunostimulatory CpG nucleic acids are incorporated herein by reference), small interfering RNA (siRNA) or microRNAs (miRNA) used to modulate gene expression, antisense oligonucleotides used to modulate gene expression, aptamers, ribozymes, a gene or gene fragment, a regulatory sequence, including analogs, derivatives, and combinations thereof. These nucleic acids may be administered neat or complexed to another entity, for example in order to facilitate their binding to and/or uptake by target tissues and/or cells.

Anti-Inflammatory Agents

Anti-inflammatory agents are agents that reduce or eliminate inflammation. They include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Other Agents

The agent may be without limitation adrenergic agent; adrenocortical steroid; adrenocortical suppressant; alcohol deterrent; aldosterone antagonist; ammonia detoxicant; amino acid; amylotropic lateral sclerosis agent; anabolic; analeptic; analgesic; androgen; anesthetic; anorectic; anorexic; anterior pituitary activator; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic including .beta.-adrenergic agonists, methylxanthines, mast cell stabilizing agents, anticholinergics, adrenocortical steroids such as glucocorticoids; anti-atherosclerotic; anticholelithic; anticholelithogenic; anticholinergic; anticoagulant; anticoccidal; anticonvulsant; antidepressant; antidiabetic; antidiarrheal; antidiuretic; antidote; antidyskinetic; anti-emetic; anti-epileptic; anti-estrogen; antifibrinolytic; antiglaucoma; antihemorrhagic; antihemorrheologic; antihistamine; antihyperlipidemic; antihyperlipoproteinemic; antihypertensive; antihypotensive; anti-infective; anti-inflammatory; antikeratinizing agent; antimigraine; antimitotic; antimycotic; antinauseant; antineutropenic; antiobsessional agent; antioxidant; antiparkinsonian; antiperistaltic; antipneumocystic; antiprostatic hypertrophy agent; antiprotozoal; antipruritic; antipsoriatic; antipsychotic; antirheumatic; antischistosomal; antiseborrheic; antisecretory; antispasmodic; antithrombotic; antitussive; anti-ulcerative; anti-urolithic; appetite suppressant; blood glucose regulator; bone resorption inhibitor; bronchodilator; carbonic anhydrase inhibitor; cardiac depressant; cardioprotectant; cardiotonic; cardiovascular agent; cerebral ischemia agent; choleretic; cholinergic; cholinergic agonist; cholinesterase deactivator; coccidiostat; cognition adjuvant; cognition enhancer; conjunctivitis agent; contrast agent; depressant; diagnostic aid; diuretic; dopaminergic agent; ectoparasiticide; emetic; enzyme inhibitor; estrogen; estrogen receptor agonist; fibrinolytic; fluorescent agent; free oxygen radical scavenger; gastric acid suppressant; gastrointestinal motility effector; geriatric agent; glucocorticoid; gonad-stimulating principle; hair growth stimulant; hemostatic; herbal active agent; histamine H2 receptor antagonists; hormone; hypocholesterolemic; hypoglycemic; hypolipidemic; hypotensive; HMGCoA reductase inhibitor; impotence therapy adjunct; inflammatory bowel disease agent; keratolytic; LHRH agonist; liver disorder agent; luteolysin; memory adjuvant; mental performance enhancer; mineral; mood regulator; mucolytic; mucosal protective agent; multiple sclerosis agent; mydriatic; nasal decongestant; neuroleptic; neuromuscular blocking agent; neuroprotective; NMDA antagonist; non-hormonal sterol derivative; nutrient; oxytocic; Paget's disease agent; plasminogen activator; platelet activating factor antagonist; platelet aggregation inhibitor; post-stroke and post-head trauma agents; progestin; prostaglandin; prostate growth inhibitor; prothyrotropin; psychotropic; radioactive agent; relaxant; rhinitis agent; scabicide; sclerosing agent; sedative; sedative-hypnotic; selective adenosine A1 antagonist; sequestering agents; serotonin antagonist; serotonin inhibitor; serotonin receptor antagonist; steroid; stimulant; suppressant; thyroid hormone; thyroid inhibitor; thyromimetic; tranquilizer; unstable angina agent; uricosuric; vasoconstrictor; vasodilator; vulnerary; wound healing agent; or xanthine oxidase inhibitor.

Subjects

The invention can be practiced in virtually any subject type that is likely to benefit from delivery of agents as contemplated herein. In some embodiments, the subject is human. Subjects also include animals such as household pets (e.g., dogs, cats, rabbits, ferrets, etc.), livestock or farm animals (e.g., cows, pigs, sheep, chickens and other poultry), horses such as thoroughbred horses, and laboratory animals (e.g., mice, rats, rabbits). Subjects also include fish and other aquatic species.

The subjects to whom the agents are delivered may be normal subjects. Alternatively they may have or may be at risk of developing a condition that can be diagnosed or that can benefit from delivery of one or more particular agents. In some embodiments, an immunomodulator is not administered to a subject receiving a multilamellar lipid vesicle having crosslinks between lipid bilayers and including one or more agents described herein. In some embodiments, a subject receiving a multilamellar lipid vesicle having crosslinks between lipid bilayers and including one or more agents described herein (e.g., one or more HPV peptides (e.g., mutant HPV peptides)) is also administered one or more immunomodulators, wherein the immunomodulator is not a CT-011 antibody. In some embodiments, some subjects may test positive for un-cleared HPV infections.

Such conditions include cancer (e.g., solid tumor cancers or non-solid cancer such as leukemias), infections (including infections localized to particular regions or tissues in the body), autoimmune disorders, allergies or allergic conditions, asthma, and transplant rejection.

Tests for diagnosing the conditions embraced by the invention are known in the art and will be familiar to the ordinary medical practitioner. These laboratory tests include without limitation, endoscopy, direct visualization, microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, and serologic tests. The medical practitioner will generally also take a full history and conduct a complete physical examination in addition to running the laboratory tests listed above.

A subject having a cancer is a subject that has detectable cancer cells. A subject at risk of developing a cancer is a subject that has a higher than normal probability of developing cancer, for example, a subject whose tissues test positive for HPV. Tests for HPV may be performed using, e.g., a Pap smear test or a HPV DNA test. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

Subjects having an infection are those that exhibit symptoms thereof including without limitation fever, chills, myalgia, photophobia, pharyngitis, acute lymphadenopathy, splenomegaly, gastrointestinal upset, leukocytosis or leukopenia, and/or those in whom infectious pathogens or byproducts thereof can be detected.

A subject at risk of developing an infection is one that is at risk of exposure to an infectious pathogen. Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery, including but not limited to abdominal surgery.

The subject may have or may be at risk of developing an infection such as a bacterial infection (e.g., a mycobacterial infection), a viral infection, a fungal infection, or a parasitic infection. In these embodiments, the vesicles may include an anti-microbial agent such as an anti-bacterial agent, an anti-viral agent, an anti-fungal agent, an anti-parasitic agent and the cell carriers (e.g., the T cells) may be genetically engineered to produce another agent useful in stimulating an immune response against the infection, or potentially treating the infection.

### Cancer

The invention contemplates administration of agents to subjects having or at risk of developing a cancer including for example a solid tumor cancer, using the vesicles of the invention. The agents may be anti-cancer agents, including chemotherapeutics, antibody based therapeutics, hormone based therapeutics, and enzyme inhibitory agents, and/or they may be immunostimulatory agents such as antigens (e.g., cancer antigens) and/or adjuvants, and/or they may be diagnostic agents (e.g., imaging agents), or any of the other agents described herein. The invention contemplates that the vesicles of the invention are able to deliver higher quantities of these agents, alone or in combination, to these subjects, and/or to allow prolonged exposure of the subject to these agents via a slow steady release profile.

The cancer may be carcinoma, sarcoma or melanoma. Sarcomas are rare mesenchymal neoplasms that arise in bone (osteosarcomas) and soft tissues (fibrosarcomas). Sarcomas include without limitation liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., not bone) Ewing's sarcoma, and primitive neuroectodermal tumor), synovial sarcoma, angiosarcomas, hemangiosarcomas, lymphangiosarcomas, Kaposi's sarcoma, hemangioendothelioma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), and chondrosarcoma. Melanomas are tumors arising from the melanocytic system of the skin and other organs. Examples of melanoma include without limitation lentigo maligna melanoma, superficial spreading melanoma, nodular melanoma, and acral lentiginous melanoma. The cancer may be a solid tumor lymphoma. Examples include Hodgkin's lymphoma, Non-Hodgkin's lymphoma, and B cell lymphoma.

The cancer may be, without limitation, cervical cancer, anal cancer, vulvar cancer, head and neck cancer, oropharyngeal cancer, penile cancer, vaginal cancer, virally induced cancer, bladder cancer, pancreatic cancer, lung cancer, liver cancer, ovarian cancer, colon cancer, stomach cancer, neuroblastoma, breast cancer, prostate cancer, renal cancer, leukemia, sarcoma, carcinoma, basal cell carcinoma, non-small cell lung carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, sarcoma, melanoma, astrocytoma, oligoastrocytoma, biliary tract cancer, choriocarcinoma, CNS cancer, larynx cancer, small cell lung cancer, non-small cell lung cancer (NSCLC), adenocarcinoma, giant (or oat) cell carcinoma, squamous cell carcinoma, oral cavity cancer, skin cancer, basal cell cancer, squamous cell cancer, testicular cancer, thyroid cancer, uterine cancer, rectal cancer, cancer of the respiratory system, cancer of the urinary system, cancer of the digestive system, bone cancer, brain cancer, colorectal cancer, connective tissue cancer, endometrial cancer, eye cancer, gastric cancer, intra-epithelial neoplasm, melanoma neuroblastoma, retinoblastoma, and rhabdomyosarcoma.

For treating one or more of these cancers, the composition of the invention may include a therapeutically effective amount of the vesicle (e.g., ICMV) including an agent that is administered to patients in need thereof. As used herein, the term “therapeutically effective amount” refers to an amount effective to achieve the desired therapeutic effect. In particular, the therapeutic effective amount avoids adverse side effects. The Examples and Figures described herein further demonstrate the effectiveness of ICMV including E6 or E7 peptide (E6-ICMV and E7-ICMV) in treating a mouse model for solid tumors.

### Infection

The invention contemplates administration of agents to subjects having or at risk of developing an infection such as a bacterial infection, a viral infection, a fungal infection, a parasitic infection or a mycobacterial infection, using the vesicles of the invention. The agents may be anti-infective agents including anti-bacterial agents, anti-viral agents, anti-fungal agents, anti-parasitic agents, and anti-mycobacterial agents), immunostimulatory agents such as antigens (e.g., microbial antigens such as bacterial antigens, viral antigens, fungal antigens, parasitic antigens, and mycobacterial antigens) and/or adjuvants, diagnostic agents (e.g., imaging agents), or any of the other agents described herein. The invention contemplates that the vesicles of the invention are able to deliver higher quantities of these agents, alone or in combination, to these subjects, and/or to allow prolonged exposure of the subject to these agents via a slow steady release profile.

The bacterial infection may be without limitation an E. coli infection, a Staphylococcal infection, a Streptococcal infection, a Pseudomonas infection, Clostridium difficile infection, Legionella infection, Pneumococcus infection, Haemophilus infection, Klebsiella infection, Enterobacter infection, Citrobacter infection, Neisseria infection, Shigella infection, Salmonella infection, Listeria infection, Pasteurella infection, Streptobacillus infection, Spirillum infection, Treponema infection, Actinomyces infection, Borrelia infection, Corynebacterium infection, Nocardia infection, Gardnerella infection, Campylobacter infection, Spirochaeta infection, Proteus infection, Bacteroides infection, H. pylori infection, or anthrax infection.

The mycobacterial infection may be without limitation tuberculosis or leprosy respectively caused by the M. tuberculosis and M. leprae species.

The viral infection may be without limitation a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza virus infection, influenza A virus infection, H1N1 (swine flu) infection, respiratory syncytial virus infection, varicella-zoster virus infections, small pox infection, monkey pox infection, SARS infection or avian flu infection.

The fungal infection may be without limitation candidiasis, ringworm, histoplasmosis, blastomycosis, paracoccidioidomycosis, crytococcosis, aspergillosis, chromomycosis, mycetoma infections, pseudallescheriasis, or tinea versicolor infection.

The parasite infection may be without limitation amebiasis, Trypanosoma cruzi infection, Fascioliasis, Leishmaniasis, Plasmodium infections, Onchocerciasis, Paragonimiasis, Trypanosoma brucei infection, Pneumocystis infection, Trichomonas vaginalis infection, Taenia infection, Hymenolepsis infection, Echinococcus infections, Schistosomiasis, neurocysticercosis, Necator americanus infection, or Trichuris trichiura infection.

### Allergy and Asthma

The invention contemplates administration of agents to subjects having or at risk of developing an allergy or asthma. The agents may be allergens, immunostimulatory agents including agents that stimulate a Th1 response, immunoinhibitory or immunosuppressant agents including agents that inhibit a Th2 response, anti-inflammatory agents, leukotriene antagonists, IL-4 muteins, soluble IL-4 receptors, anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and other downregulators of IgE such as but not limited to anti-IgE, cytokines such as Th1 cytokines such as IL-12 and IFN-gamma, steroids including corticosteroids such as prednisolone, and/or they may be diagnostic agents (e.g., imaging agents), or any of the other agents described herein. The invention contemplates that the vesicles of the invention are able to deliver higher quantities of these agents, alone or in combination, to these subjects, and/or to allow prolonged exposure of the subject to these agents via a slow steady release profile.

An allergy is an acquired hypersensitivity to an allergen. Allergic conditions include but are not limited to eczema, allergic rhinitis or coryza, hay fever, bronchial asthma, urticaria (hives) and food allergies, and other atopic conditions. Allergies are generally caused by IgE antibody generation against harmless allergens. Asthma is a disorder of the respiratory system characterized by inflammation, narrowing of the airways and increased reactivity of the airways to inhaled agents. Asthma is frequently, although not exclusively, associated with atopic or allergic symptoms.

### Autoimmune Disease

The invention contemplates administration of agents to subjects having or at risk of developing an autoimmune disease or disorder. The agents may be immunoinhibitory or immunosuppressant agents including those that inhibit a Th1 response, immunostimulatory agents that stimulate a Th2 response, cytokines such as IL-4, IL-5 and IL-10, anti-inflammatory agents, and/or they may be diagnostic agents (e.g., imaging agents), or any of the other agents described herein. The invention contemplates that the vesicles of the invention are able to deliver higher quantities of these agents, alone or in combination, to these subjects, and/or to allow prolonged exposure of the subject to these agents via a slow steady release profile.

Autoimmune disease is a class of diseases in which a subject's own antibodies react with host tissue or in which immune effector T cells are autoreactive to endogenous self peptides and cause destruction of tissue. Thus an immune response is mounted against a subjects own antigens, referred to as self antigens. Autoimmune diseases are generally considered to be Th1 biased. As a result, induction of a Th2 immune response or Th2 like cytokines can be beneficial.

Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus.

### Transplant Therapy

The invention contemplates administration of agents to subjects undergoing a cell or organ transplant. The agents may be immunoinhibitory or immunosuppressant agents, anti-inflammatory agents, and/or they may be diagnostic agents (e.g., imaging agents), or any of the other agents described herein. The invention contemplates that the vesicles of the invention are able to deliver higher quantities of these agents, alone or in combination, to these subjects, and/or to allow prolonged exposure of the subject to these agents via a slow steady release profile.

The compositions and methods provided herein may also be used to modulate immune responses following transplant therapy. Transplant success is often limited by rejection of the transplanted tissue by the body's immune system. As a result, transplant recipients are usually immunosuppressed for extended periods of time in order to allow the transplanted tissue to survive. The invention contemplates delivery of immunomodulators, and particularly immunoinhibitory agents, to transplant sites in order to minimize transplant rejection. Thus, the invention contemplates administration to subjects that are going to undergo, are undergoing, or have undergone a transplant.

The foregoing lists are not intended to be exhaustive but rather exemplary. Those of ordinary skill in the art will identify other examples of each condition type that are amenable to prevention and treatment using the methods of the invention.

### Effective Amounts, Regimens, Formulations

The agents are administered in the form of stabilized MLVs including one or more conjugated agents (e.g., an antigen such as a full-length protein antigen), and in effective amounts. An effective amount is a dosage of the agent sufficient to provide a medically desirable result. The effective amount will vary with the desired outcome, the particular condition being treated or prevented, the age and physical condition of the subject being treated, the severity of the condition, the duration of the treatment, the nature of the concurrent or combination therapy (if any), the specific route of administration and like factors within the knowledge and expertise of the health practitioner. It is preferred generally that a maximum dose be used, that is, the highest safe dose according to sound medical judgment.

For example, if the subject has a tumor, an effective amount may be that amount that reduces the tumor volume or load (as for example determined by imaging the tumor). Effective amounts may also be assessed by the presence and/or frequency of cancer cells in the blood or other body fluid or tissue (e.g., a biopsy). If the tumor is impacting the normal functioning of a tissue or organ, then the effective amount may be assessed by measuring the normal functioning of the tissue or organ.

In some instances the effective amount is the amount required to lessen or eliminate one or more, and preferably all, symptoms. For example, in a subject having an allergy or experiencing an asthmatic attack, an effective amount of an agent may be that amount that lessens or eliminates the symptoms associated with the allergy or the asthmatic attack. They may include sneezing, hives, nasal congestion, and labored breathing. Similarly, in a subject having an infection, an effective amount of an agent may be that amount that lessens or eliminate the symptoms associated with the infection. These may include fever and malaise. If the agent is a diagnostic agent, an effective amount may be an amount that allows visualization of the body region or cells of interest. If the agent is an antigen, the effective amount may be that amount that triggers an immune response against the antigen and preferably provides short and even more preferably long term protection against the pathogen from which the antigen derives. It will be understood that in some instances the invention contemplates single administration of an agent and in some instances the invention contemplates multiple administrations of an agent. As an example, an antigen may be administered in a prime dose and a boost dose, although in some instances the invention provides sufficient delivery of the antigen, and optionally an adjuvant, that no boost dose is required.

The invention provides pharmaceutical compositions. Pharmaceutical compositions are sterile compositions that include the vesicles of the invention and preferably agent(s), preferably in a pharmaceutically-acceptable carrier. The term “pharmaceutically-acceptable carrier” means one or more compatible solid or liquid filler, diluents or encapsulating substances which are suitable for administration to a human or other subject contemplated by the invention.

The term “carrier” denotes an organic or inorganic ingredient, natural or synthetic, with which vesicles and preferably agent(s) are combined to facilitate administration. The components of the pharmaceutical compositions are comingled in a manner that precludes interaction that would substantially impair their desired pharmaceutical efficiency.

Suitable buffering agents include acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); Phosphate buffered saline (PBS), and parabens (0.01-0.25% W/V).

Unless otherwise stated herein, a variety of administration routes are available. The particular mode selected will depend, of course, upon the particular active agent selected, the particular condition being treated and the dosage required for therapeutic efficacy. The methods provided, generally speaking, may be practiced using any mode of administration that is medically acceptable, meaning any mode that produces effective levels of a desired response without causing clinically unacceptable adverse effects. One mode of administration is a parenteral route. The term “parenteral” includes subcutaneous injections, intravenous, intramuscular, intraperitoneal, intra sternal injection or infusion techniques. Other modes of administration include oral, mucosal, rectal, vaginal, sublingual, intranasal, intratracheal, inhalation, ocular, and transdermal.

For oral administration, the compounds can be formulated readily by combining the vesicles with pharmaceutically acceptable carriers well known in the art. Such carriers enable formulation as tablets, pills, dragees, capsules, liquids, gels, syrups, slurries, films, suspensions and the like, for oral ingestion by a subject to be treated. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). Optionally the oral formulations may also be formulated in saline or buffers for neutralizing internal acid conditions or may be administered without any carriers.

Pharmaceutical preparations which can be used orally include push-fit capsules made of gelatin, as well as soft, sealed capsules made of gelatin and a plasticizer, such as glycerol or sorbitol. The push-fit capsules can contain the vesicles suspended in suitable liquids, such as aqueous solutions, buffered solutions, fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

When it is desirable to deliver the compositions of the invention systemically, they may be formulated for parenteral administration by injection, e.g., by bolus injection or continuous infusion. Formulations for injection may be presented in unit dosage form, e.g., in ampoules or in multi-dose containers. Pharmaceutical parenteral formulations include aqueous solutions of the ingredients. Aqueous injection suspensions may contain substances which increase the viscosity of the suspension, such as sodium carboxymethyl cellulose, sorbitol, or dextran. Alternatively, suspensions of vesicles may be prepared as oil-based suspensions. Suitable lipophilic solvents or vehicles include fatty oils such as sesame oil, or synthetic fatty acid esters, such as ethyl oleate or triglycerides.

Alternatively, the vesicles may be in powder form or lyophilized form for constitution with a suitable vehicle, e.g., sterile pyrogen-free water, before use.

The compositions may also be formulated in rectal or vaginal compositions such as suppositories or retention enemas, e.g., containing conventional suppository bases such as cocoa butter or other glycerides.

### Methods of Treatment

The invention provides methods of treatment that may be used to treat subjects who are suffering from diseases and disorders, e.g., cancers (e.g., a HPV-positive cancer) and infections (e.g., a HPV infection). In some embodiments, methods of the invention include administering to a subject in need thereof, a therapeutically effective amount of a composition described herein (e.g., a composition containing a multilamellar lipid vesicle having crosslinks between lipid bilayers and including one or more mutant HPV peptides, wherein at least one of the one or more mutant HPV peptides is conjugated to a lipid of the multilamellar lipid vesicle). In some embodiments, an immunomodulator is not administered to the subject.

In some embodiments, methods of the invention include administering to a subject in need thereof, a therapeutically effective amount of a composition described herein and one or more immunomodulators described herein, wherein the immunomodulator is not a CT-011 antibody.

In some embodiments, methods of the invention include a multilamellar lipid vesicle having crosslinks between lipid bilayers and including one or more mutant HPV peptides, and one or more immunomodulators described herein, wherein the immunomodulator is not a CT-011 antibody.

In some embodiments, the composition and the one or more immunomodulators are administered substantially simultaneously.

In some embodiments, the composition and the one or more immunomodulators are administered separately. In some embodiments, the composition is administered first, followed by administering of the one or more immunomodulators. In some embodiments, the one or more immunomodulators are administered first, followed by administering of the composition.

### In Vitro Use

The invention further contemplates in vitro applications such as cell culturing and tissue engineering, that require or for which it would be more convenient to have a constant source of one or more agents such as but not limited to cell growth factors.

### Kits

The invention further contemplates kits including the vesicles of the invention. The vesicles may include one or more agents of interest. The kits may further include one or more agents of interest to be incorporated into the vesicles. These kits may also include written materials such as instructions for use of the vesicles. The vesicles may be provided in a buffer or in a lyophilized form, preferably with a sucrose-containing excipient.

The invention also contemplates kits including the various substrates, reagents and catalysts required for synthesizing the vesicles of the invention. Such kits may include for example lipids such as those described herein, functionalized components of a lipid bilayer such as functionalized lipids, one or more crosslinkers such as membrane permeable crosslinkers, multivalent cations such as divalent cations, and the like. These kits may also include written materials such as instructions for synthesizing the vesicles. The kits may also include the agents of interest.

In some embodiments, kits of the invention contain (i) a multilamellar lipid vesicle having crosslinks between lipid bilayers and including one or more agents (e.g., HPV peptides (e.g., mutant HPV peptides)), (ii) one or more immunomodulators, and (iii) instructions for administering (i) and (ii) to a subject having a disease (e.g., a cancer (e.g., a HPV-positive cancer), an infection (e.g., a HPV infection)), wherein the agent is not a terminal-cysteine-bearing antigen, and wherein the immunomodulator is not a CT-011 antibody.

In some embodiments, kits of the invention contain (i) a multilamellar lipid vesicle having crosslinks between lipid bilayers and including one or more agents (e.g., HPV peptides (e.g., mutant HPV peptides)), and (ii) instructions for administering (i) with one or more immunomodulators to a subject having a disease (e.g., a cancer (e.g., a HPV-positive cancer), an infection (e.g., a HPV infection)), wherein the agent is not a terminal-cysteine-bearing antigen, and wherein the immunomodulator is not a CT-011 antibody.

In some embodiments, kits of the invention contain (i) one or more immunomodulators, and (ii) instructions for administering (i) with a multilamellar lipid vesicle having crosslinks between lipid bilayers and including one or more agents (e.g., HPV peptides (e.g., mutant HPV peptides)) to a subject having a disease (e.g., a cancer (e.g., a HPV-positive cancer), an infection (e.g., a HPV infection)), wherein the agent is not a terminal-cysteine-bearing antigen, and wherein the immunomodulator is not a CT-011 antibody.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### Examples

### Example 1. Synthesis of ICMV with Conjugated Agents

Materials:

(a) DOPC (5 mg/ml in CHCl₃—Avanti Polar Lipids #850375)
(b) MPB (10 mg/ml in CHCl₃—Avanti Polar Lipids #870012)
(c) 3M-052 (0.1 mg/ml in CHCl₃) or MPLA/GLA (0.5 mg/ml in MeOH)
(d) (PEG 2 k)-SH (100 mg/ml in H₂O—Laysan Bio #MPEG-SH-2000)

### (e) Dithiothreitol (150 mM in H₂O—Sigma Aldrich #43819)

(f) CaCl₂ (200 mM in H₂O—Fischer Scientific #BP510)
(g) Agent at appropriate concentration in 20 mM bis-Tris-propane, pH 7.0 (bTp—Sigma Aldrich #B4679)
(h) Traut's reagent (10 mg/ml in bTp—Thermo Scientific #26101)

Procedure:

DOPC (500 μL), MPB (325 μL), and optional adjuvant components were dried in a scintillation vial under vacuum for at least 12 hours, followed by resuspension in bTp (1 mL) by vortexing at 3000 rpm for 10 seconds every 10 minutes for 1 hour at room temperature. The lipid/adjuvant mixture was fluidized in a Microfluidics LV-1 by loading 1 mL of solution, discarding the product, then loading 3 mL of bTp and collecting the product (assuming 2 mL dead volume in fluidizer). In parallel to fluidizing the lipid/adjuvant mixture, agent was treated with 40-80 molar ratio of Traut's reagent for 1 hour at room temperature. The fluidized lipid/adjuvant mixture was then incubated at 37° C. for between 12 and 24 hours with the functionalized agent. The ICMVs were crosslinked and sealed by adding DTT (20 μL) and CaCl₂ (200 ρL/mL total volume) and placing at 37° C. for 1 hour. The vesicles were collected by centrifugation at 21000×g for 5 minutes, and resuspended in 1 mL water. The vesicles were PEGylated by addition of PEG-SH (50 μL) and incubation at 37° C. for 1 hour. The PEGylated vesicles were collected by centrifugation at 21000×g for 5 minutes, and resuspended in PBS, followed by extrusion through a 0.2 μm membrane nine times.

For long term storage a portion or all of the vesicles may be centrifuged at 21000×g for 5 minutes, and resuspended in PBS+40% sorbitol.

Possible ranges and desired ranges for the variables in the above procedure are shown in Table 1.

TABLE 1
Ranges of Variables in ICMV Preparation

| Parameter | Possible range | Desired range |
|---|---|---|
| Fluidizer pressure (psi) | 10000-40000 | 25000-35000 |
| Fluidizer passes | 1-4 | 1-2 |
| Fluidizer collection volume (ml) | 1-13 | 3-4 |
| Functionalization time (h) | 0-6 | 0.5-1.5 |
| Lipid/protein mixing time (h) | 0.5-48 | 6-24 |
| Crosslinking time (h) | 0.5-6 | 1-2 |
| PEGylation time (h) | 0.5-6 | 1-3 |
| Centrifuge speed (xg) | 1000-22000 | 3000-21000 |
| Centrifuge time (min) | 1-30 | 5-15 |
| Sorbitol content (w/v %) | 1-80 | 35-45 |
| Extrusion passes | 1-30 | 5-15 |
| DTT volume (ul/ml lipids) | 10-100 | 20-30 |
| CaCl2 volume (ul/ml total) | 100-1000 | 150-300 |

### Example 2. Optimization of Agent Functionalization

Ovalbumin with differing molar equivalents of Traut's reagent produced different functionalization ratios. The amount of free thiol was determined using Elman's assay.

### Materials:

Ellman's reagent (Pierce); Ellman's reaction buffer is 0.1M sodium phosphate, 1 mM EDTA, pH 8.0; Cysteine hydrochloride monohydrate (Pierce)

### Protocol:

Ellman's reagent (4 mg) was dissolved in Ellman's reaction buffer (1 mL). Cysteine standards and samples were added to a 96 well plate in triplicate at 20 uL/well. Ellman's reagent (20 μL per well) and PBS (60 μL per well) was added and incubated for 15 minutes. Absorbance was read at 412 nm fixed wavelength (reference wavelength 540 nm). The results are shown in Table 2.

TABLE 2
Functionalization of Ovalbumin

| | | | | | | mol SH/ |
|---|---|---|---|---|---|---|
| mol | | no | | nmol | nmol | mol |
| Traut | fluorescence | background | mg/ml | Ova/ml | SH/ml | Ova |
| 20 | 45725 | 43333.5 | 3.240 | 71.993 | 25.272 | 0.3510 |
| 40 | 46775 | 44383.8 | 3.324 | 73.860 | 47.726 | 0.6462 |
| 60 | 44278 | 41887.2 | 3.124 | 69.422 | 67.275 | 0.9691 |
| 80 | 44800 | 42408.5 | 3.166 | 70.348 | 77.578 | 1.1028 |

When functionalizing Ovalbumin with 20 molar equivalents of Traut's reagent, each molecule of Ovalbumin had an average of 0.35 molecules of added sulfur, while at 80 molar equivalents each molecule of Ovalbumin had an average of 1.1 molecules of added sulfur.

Other thiolation reagents have been shown to result in varying levels of thiolation. The results are shown in Table 3.

TABLE 3
Other Thiolation Reagents

| Functionalizer | Molar Equivalents | mol SH/mol Ova |
|---|---|---|
| SATA | 10 | 1.89 |
| SATA | 50 | 11.55 |
| SAT-PEG | 10 | 0.54 |
| SAT-PEG | 50 | 6.09 |

No increase in protein encapsulation seen by significant increases in thiolation. Therefore, desired functionalization is achieved at between 0.5 and 2 mol SH/mol protein.

### Example 3. Optimization of Agent Conjugation

Experiments were conducted to determine if increasing the time allowed for agent conjugation to fluidized lipids would improve encapsulation efficiency. The results are shown in Table 4.

TABLE 4
Optimization of Agent Conjugation Time

| | | no | | Encapsulation |
|---|---|---|---|---|
| Sample | fluorescence | background | mg/ml | efficiency |
| 1 hour | 7751 | 6701 | 0.387 | 14.1% |
| 24 hour | 9778 | 8728 | 0.569 | 21.5% |

Reactions with thiol-containing agents with the ICMVs are usually limited to 1 hour because of the high reactivity of the maleimide groups on ICMVs to free thiol groups, but because the functionalization of the large protein molecules is low it was found that providing more time for this reaction to occur increased the loading efficiency of the protein by 47%. Accordingly, the desired encapsulation efficiency was achieved with a 24 hour incubation period to allow for increased conjugation of the agent.

### Example 4. Optimization of Fluidization Step

Experiments were conducted to ensure the most material would be recovered from the fluidizer without large dilution. The results are shown in Table 5.

TABLE 5
Optimization Fluidization

| | | no | |
|---|---|---|---|
| Sample | fluorescence | background | mg |
| 1 mL load | 8723 | 8229 | 0.576 |
| 1 mL push | 15537 | 15043 | 1.053 |
| 1 mL collect | 17426 | 16932 | 1.185 |
| Left in fluidizer | 9605 | 9111 | 0.638 |
| 1 mL load | 8890 | 8396 | 0.588 |
| 2 mL push | 13492 | 12998 | 1.820 |
| 1 mL collect | 11632 | 11138 | 0.780 |
| Left in fluidizer | 6007 | 5513 | 0.386 |

Flushing of the fluidizer with 3 mL of buffer after loading 1 mL of lipid sample results in the recovery of most of the material without wasting buffer.

### Example 5. Storage Optimization

When compared to PBS solutions containing sucrose, free PEG, or polysorbate 20 (PS-20), those containing 40% sorbitol were better able to maintain particle size in solution over time. This was determined by a lack of aggregates or shift in particle diameter over the course of 12 days at 4° C. Upon subsequent study, PBS+40% sorbitol has maintained particle size and protein loading for over 7 months.

### Example 6. Cloning of DNA Plasmid Constructs of HPV-16 E6 and/or E7 Peptides into Expression Vectors

pVEXK His6x-HPV-16 E6 and pVEXK His6x-HPV-16 E7

For each of the nucleic acid sequences encoding the HPV-16 E6 and E7 peptides, a sequence containing a 5′ terminal NdeI site was added upstream of the ATG start codon. A sequence containing a 3′ terminal EcoRI site was added downstream of the 3′ terminal TGA translation termination codon. The nucleic acid sequence of the E6 or E7 protein was then cloned into pVEXK His6x expression vector using NdeI and EcoRI as restriction sites.

pVEXK His6x-HPV-16 E6-E7

For the sequence encoding the HPV-16 E6-E7 fusion protein, a sequence containing a 5′ terminal AgeI site was added upstream of the ATG start codon. A sequence containing a 3′ terminal EcoRI site was added downstream of the 3′ terminal TGA translation termination codon. The nucleic acid sequence of the E6-E7 fusion protein was then cloned into pVEXK His6x expression vector using AgeI and EcoRI as restriction sites.

### Example 7. Immunogenicity Induced by E6-ICMV and E7-ICMV

Immunogenicity induced by E6-ICMV and E7-ICMV was measured by quantifying the percentage of IFNγ secreting cells among E6/E7-specific CD8 T cells.

Mice were inoculated on the left dorsal flank with 50,000 TC-1 tumor cells on Day 0. Three doses of treatment were given weekly starting on Day 7. Mice were treated with 10 μg HPV-16 E7 in ICMVs (E7-ICMV) and 10 μg HPV-16 E6 in ICMVs (E6-ICMV) via subcutaneous injection, or the same amounts of unformulated soluble E7 and E6 proteins in PBS.

Mice were bled 1 week after injection and immunogenicity was tested by quantifying the percentage of IFNγ secreting E6/E7-specific CD8 T cells. Blood cells were collected then stimulated with E749-57 (RAHYNIVTF (SEQ ID NO: 11)) peptide or E648-57 (EVYDFAFRDL (SEQ ID NO: 12)) peptide. The percentage of IFNγ secreting E6/E7-specific CD8 T cells was measured using intracellular cytokine staining assay.

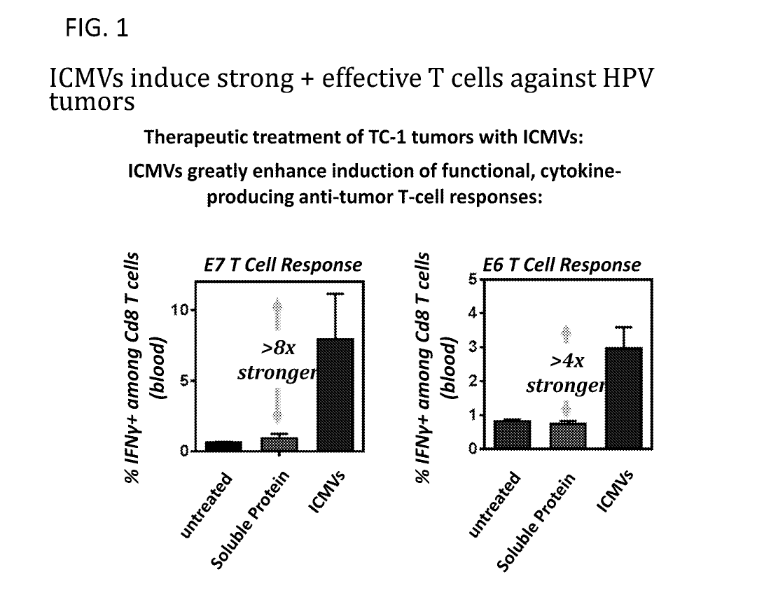

FIG. 1 shows the treatment with E6-ICMV+E7-ICMV produced the highest percentage of IFNγ⁺ CD8⁺ T cells.

### Example 8. Treatment of Solid Tumors in Mice Using E6-ICMV and E7-ICMV

ICMVs including an E6 or E7 peptide of HPV-16 (E6-ICMV and E7-ICMV) were tested for their effectiveness in treating a mouse model for solid tumors. Mice were inoculated on the left dorsal flank with 50,000 TC-1 tumor cells on Day 0. Three doses of treatment were given weekly starting on Day 7. Mice were treated with 10 μg HPV-16 E7 in ICMVs (E7-ICMV) and 10 μg HPV-16 E6 in ICMVs (E6-ICMV) via subcutaneous injections, or the same amounts of unformulated soluble E7 and E6 proteins in PBS.

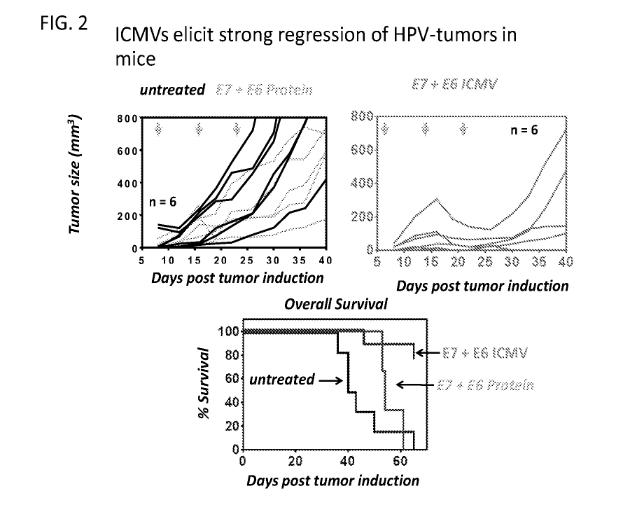

FIG. 2 shows the quantification of tumor volume measured using the equation V=L×W²/2. Only mice treated with E6 and E7 in ICMVs showed strong tumor regression and prolonged survival.

### Example 9. ICMV Therapy in Combination with an Immunomodulator

Mice were inoculated on the left dorsal flank with 50,000 TC-1 tumor cells on Day 0. Three doses of treatment were given weekly starting on Day 7.

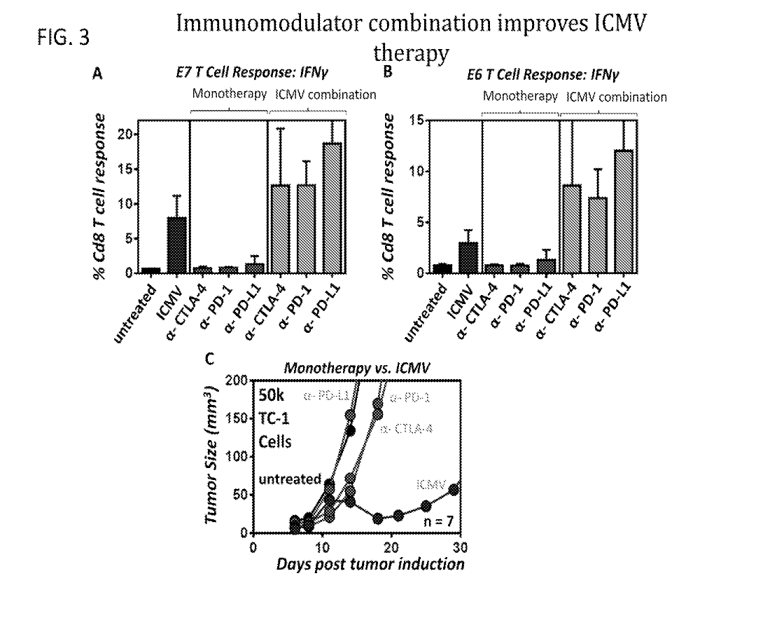

FIGS. 3A and 3B show the immunogenicity induced by E6-ICMV and E7-ICMV with and without an immunomodulator selected from anti-CTLA-4 antibody (aCTLA-4), anti-PD-1 antibody (aPD-1), and anti-PD-L1 antibody (aPD-L1). Mice were treated with 1) 10 μg HPV-16 E7 in ICMVs (E7-ICMV)+10 μg HPV-16 E6 in ICMVs (E6-ICMV) alone (subcutaneous injection), or 2) 230 μg per dose of anti-CTLA-4 antibody (aCTLA-4), anti-PD-1 antibody (aPD-1), or anti-PD-L1 antibody (aPD-L1) alone (intraperitoneal (i.p.) injection), or 3) E6-ICMV+E7-ICMV+aCTLA-4 or aPD-1 or aPD-L1.

Mice were bled 1 week after injection and immunogenicity was tested by quantifying the percentage of IFNγ secreting E6/E7-specific CD8 T cells. FIGS. 3A and 3B show the treatment with combination of E6-ICMV+E7-ICMV+aCTLA-4 or aPD-1 or aPD-L1 outperformed the treatment with ICMV monotherapy or immunomodulator monotherapy and produced the highest percentage of IFNγ⁺ CD8⁺ cells.

FIG. 3C shows the reduction in tumor size induced by ICMV monotherapy (E6-ICMV and E7-ICMV) and immunomodulator monotherapy (anti-CTLA-4 antibody (aCTLA-4), anti-PD-1 antibody (aPD-1), or anti-PD-L1 antibody (aPD-L1)). FIG. 3C shows the tumor volume measured using the equation V=L×W²/2. Mice treated with ICMV monotherapy (E6-ICMV+E7-ICMV) show strong tumor regression than immunomodulator monotherapy.

### Example 10. Treatment of Larger Solid Tumors in Mice Using E6-ICMV and E7-ICMV with and without the Immunomodulator Anti-PD-1 Antibody

ICMVs including an E6 or E7 peptide of HPV-16 (E6-ICMV and E7-ICMV) were tested for their effectiveness in treating a mouse model for large solid tumors with and without anti-PD-1 antibody. Mice were inoculated on the left dorsal flank with an increased tumor burden of 1,000,000 TC-1 tumor cells per mouse. FIG. 4 shows that combination of E6-ICMV+E7-ICMV+anti-PD-1 antibody has better therapeutic benefit than E6-ICMV+E7-ICMV in mice with higher tumor burdens.

### Example 11. Treatment of Solid Tumors in Mice Using E6-ICMV and E7-ICMV with and without the Immunomodulator Cyclophosphamide

Mice were inoculated on the left dorsal flank with 50,000 TC-1 tumor cells on Day 0. Three doses of treatment were given weekly starting on Day 7. Mice were treated with 1) 10 μg E7-ICMV+10 μg E6-ICMV alone (subcutaneous injection), or 2) cyclophosphamide (CPM) alone (50 mg/kg/dose, i.p. injection), or 3) E6-ICMV+E7-ICMV+CPM.

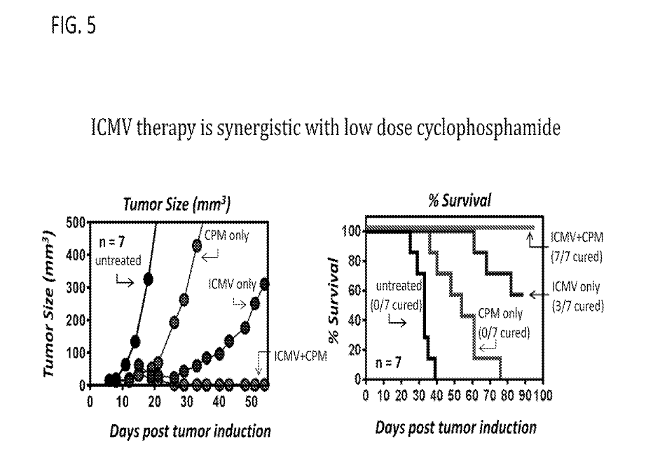

FIG. 5 shows that the therapeutic benefit of E6-ICMV+E7-ICMV is greater than CPM alone as stronger tumor regression and prolongation of survival is seen in the E6-ICMV+E7-ICMV treated group. The combination of E6-ICMV+E7-ICMV+CPM is the most potent, resulting in eradication of tumors and 100% cure rates.

### Example 12. ICMV Therapy Elicits Long-Lived T-Cell Memory and Protection

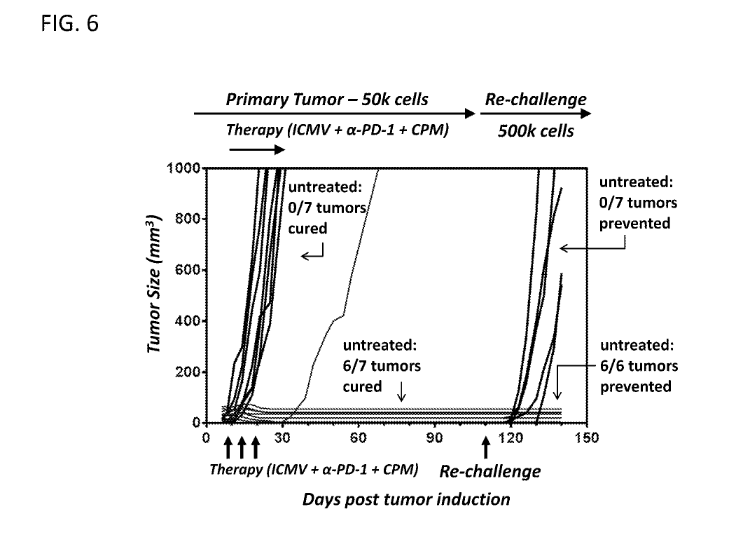

Mice were inoculated in the left dorsal flank with TC-1 tumor cells on Day 0. Three doses of E6-ICMV+E7-ICMV+anti-PD-1 antibody+cyclophosphamide (CPM) treatment were given weekly starting on Day 7. FIG. 6 shows that the treatment resulted in cures in 6 out of 7 mice. To test if long term antigen-specific memory T cells are present and functional in these cured mice, 500 k TC1 tumor cells were injected into the flank at about 110 days after the first dose. The E6-ICMV+E7-ICMV+anti-PD-1 antibody+CPM treatment successfully prevented tumor regrowth in 100% of the 6 cured mice after the re-challenge (FIG. 6).

### Example 13. ICMV Therapy in Combination with Cyclophosphamide Controls Growth of Large Established Tumors

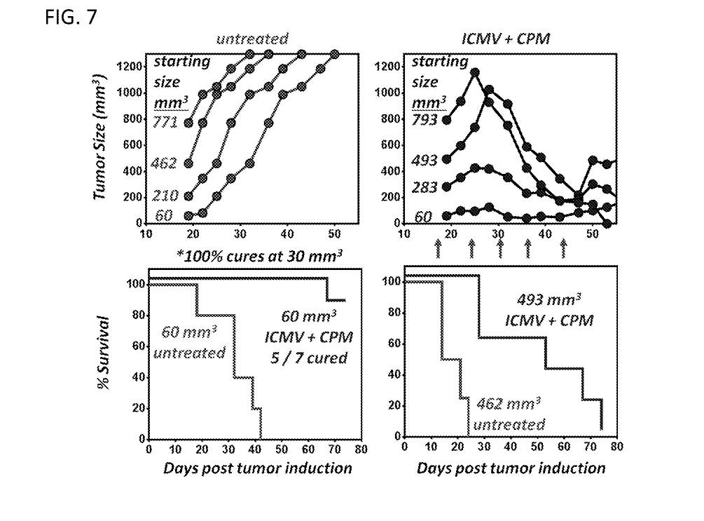

Mice were inoculated in the left dorsal flank with TC-1 tumor cells and tumors of various sizes from 60 mm³ to 793 mm³ were treated with 5 doses of E6-ICMV+E7-ICMV+cyclophosphamide (CPM). FIG. 7 shows that the treatment resulted tumor regression (top) and prolonged survival (bottom) in all mice. This demonstrates the potency of the combination of E6-ICMV+E7-ICMV+CPM. The treatment can attack tumors that are much larger than TC1 tumors reported in the literature.

### Example 14. ICMV Therapy in Combination with Cyclophosphamide Cures Tongue Tumors

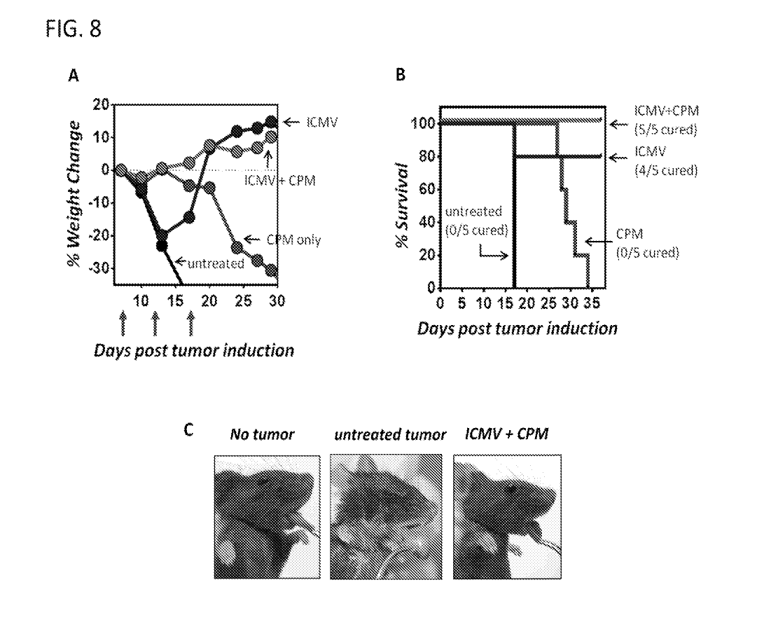

Mice were inoculated in the tongue with 50,000 TC-1 tumor cells on Day 0. Three weekly doses of E6-ICMV+E7-ICMV+cyclophosphamide (CPM) (subcutaneous injection), E6-ICMV+E7-ICMV, or CPM were given starting on Day 7. Body weight was used as an indicator for tumor size in the tongue as food intake is directly related to tumor size. FIGS. 8A and 8B show that the treatment resulted in weight gain and prolonged survival, respectively, in all mice treated with E6-ICMV+E7-ICMV+CPM or E6-ICMV+E7-ICMV. FIG. 8C shows the tumor regression and disappearance of tongue tumor in mice that received the E6-ICMV+E7-ICMV+CPM treatment. The results show that the treatment can treat tumor in the tongue with similar trends to tumors in the flank: E6-ICMV+E7-ICMV+CPM can cure tumors, E6-ICMV+E7-ICMV is more potent than cyclophosphamide alone. This demonstrates the immune response elicited by the treatment can treat tumors distal from the injection size.

### Example 15. ICMV Therapy in Combination with an Indoleamine-2,3-Dioxygenase (IDO) Inhibitor can Improve Therapeutic Benefit

Mice were inoculated in the left dorsal flank with 1,000,000 TC-1 tumor cells on Day 0. Three doses of treatment were given weekly starting on Day 7. Mice were treated with 1) 10 μg HPV-16 E7 in ICMVs (E7-ICMV)+10 μg HPV-16 E6 in ICMVs (E6-ICMV) (subcutaneous injection) or 2) E6-ICMV+E7-ICMV+the IDO inhibitor 1-methyltryptophan (8 mg daily via oral gavage). FIG. 9 shows the tumor volume measured using the equation V=L×W²/2. Mice treated with E6-ICMV+E7-ICMV+IDO inhibitor exhibited faster tumor regression compared to mice treated with E6-ICMV+E7-ICMV, demonstrating that IDO inhibitors can be used to enhance the effects of ICMV therapy.

### Example 16. Treatment of Solid Tumors in Mice Using E6-ICMV and/or E7-ICMV with and without an Adjuvant

An ICMV including an E7 peptide of HPV-16 (E7-ICMV) was tested for its effectiveness in treating a mouse model for solid tumors. 250,000 tissue culture-1 (TC-1) tumor cells were injected into the left flank of C57BL/6 mice on Day 0. On Day 6 and Day 19, mice were immunized with a dose of HPV E7 whole protein encapsulated in ICMV (E7-ICMV) with no adjuvant or with monophosphoryl lipid A (MPLA) as adjuvant. Each dose of E7-ICMV contained 5-10 μg of E7 protein and 6-10 μg of MPLA.

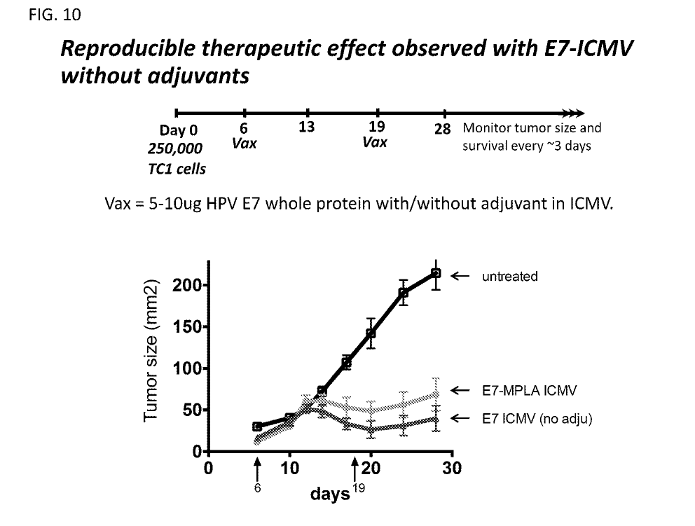

Tumor size was monitored twice a week. FIG. 10 shows that both E7-ICMV immunized groups (E7-ICMV (no adju.) and E7-MPLA ICMV) showed similar rates of tumor regression, indicating that E7-ICMV in an immunogenic composition that without an adjuvant, also can elicit effective therapeutic immune responses.

Following the same protocol, an ICMV including an E6 peptide of HPV-16 (E6-ICMV) with and without an adjuvant could be tested for its effectiveness in treating a mouse model for solid tumors.

In addition, following the same protocol, a combination therapy including both E6-ICMV and E7-ICMV with and without an adjuvant could be tested for its effectiveness in treating a mouse model for solid tumors.

### Example 17. Treatment of Solid Tumors in Mice Using E6-ICMV and/or E7-ICMV with and without a Potentiating Agent

E6-ICMV or E7-ICMV in combination with a potentiating agent may be tested for its effectiveness in treating a mouse model for solid tumors. The potentiating agent may be a molecule (e.g., a small, organic molecule), peptide, protein (e.g., an antibody or an antigen), or a drug that can enhance or augment the effectiveness of E6-ICMV or E7-ICMV in treating a disease. Potentiating agents include, but are not limited to, anticancer agents, immunostimulatory agents, cancer antigens, anti-infective agents, anti-viral agents, and anti-fungal. Examples of potentiating agents are described above. Preferably, the potentiating agent is an antibody. Following similar protocol as the one described in the Example 16, mice injected with TC-1 tumor cells may be immunized with one or more doses of E6-ICMV or E7-ICMV with or without the potentiating agent. Tumor size may be monitored in both treatment groups to compare their effectiveness in promoting tumor regression.

Following the same protocol, a combination therapy including both E6-ICMV and E7-ICMV with and without a potentiating agent could be tested for its effectiveness in treating a mouse model for solid tumors.

### Example 18. Treatment of Solid Tumors in Mice Using E6-ICMV and E7-ICMV

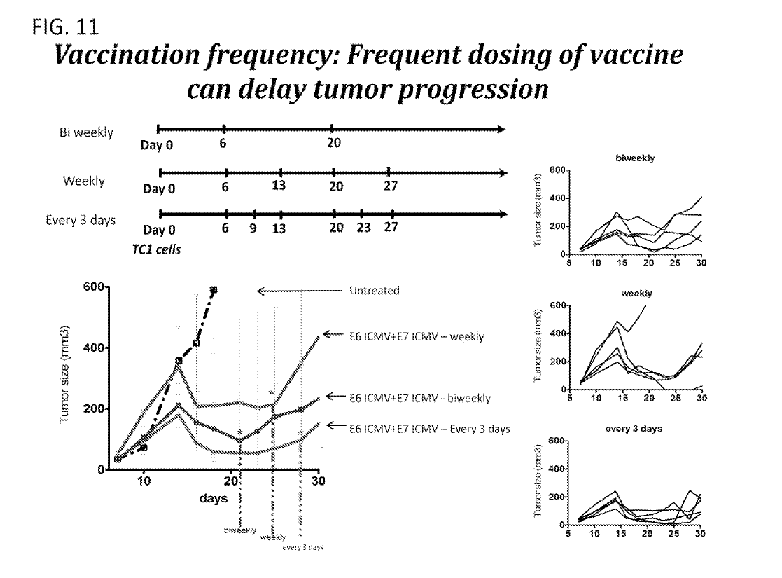

An ICMV including an E6 peptide of HPV-16 (E6-ICMV) and E7-ICMV were used in a combination therapy to test its effectiveness in treating a mouse model for solid tumors. 250,000 tissue culture-1 (TC-1) tumor cells were injected into the left flank of C57BL/6 mice on Day 0. Following tumor injection, mice were immunized with a dose of HPV E6 and HPV E7 protein encapsulated into separate ICMV (5-10 μg of each antigen per dose) biweekly, weekly, or every three days starting on Day 6. FIG. 11 shows tumor size until Day 30 after tumor implantation. The combination therapy of E6-ICMV+E7-ICMV enhanced tumor regression more effectively with more frequent dosing.

### Example 19. Treatment of Solid Tumors in Mice Using E6-ICMV, E7-ICMV, and Peptide Vaccines

A combination therapy containing an ICMV including an E6 peptide of HPV-16 (E6-ICMV) and E7-ICMV was compared with E6 and E7 peptide vaccines in treating a mouse model for solid tumors. 50,000 tissue culture-1 (TC-1) tumor cells were injected into the left flank of C57BL/6 mice on Day 0. Following tumor injection, mice were immunized with (1) a dose of HPV-16 E6 peptide+E7 peptide (5-10 μg each), (2) E6 and E7 CD8 epitope (100 μg each)+50 μg R848 (resiquimod, an immune response modifier), (3) E6 and E7 CD8 epitope (100 μg each)+50 μg polyIC, or (4) E6-ICMV+E6-ICMV (5-10 μg each, no adjuvant) on Day 8, 15, and 22. Tumor size was monitored in all treatment groups to compare their effectiveness in promoting tumor regression. The combination therapy of E6-ICMV+E7-ICMV (therapy (4)) enhanced tumor regression more effectively than two therapies using E6 and E7 CD8 epitope (therapies (2) and (3)) and one therapy using E6 and E7 peptide vaccines (therapy (1)).

### Example 20. Immunogenicity Induced by E7 Peptide Encapsulated in ICMV with Adjuvants

Immunogenicity induced by E7 peptide was measured by quantifying the percentage of tetramer positive (H2-D^{b} E7₄₉₋₅₇ (RAHYNIVTF (SEQ ID NO: 11))-containing tetramers) cells among CD8-positive T cells in mice.

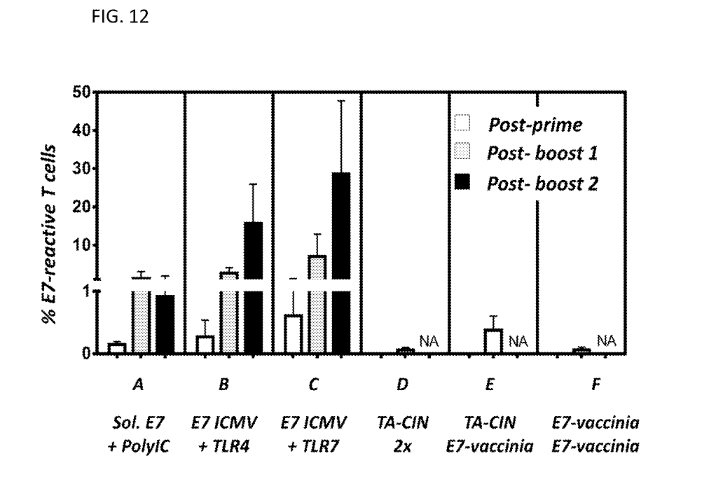

Mice were vaccinated either on Days 0, 28, and 56 (Panels A-C of FIG. 12) or Days 0 and 21 (Panels D-F of FIG. 12) and tetramer-specific CD8 T cells (H2-D^{b} E7₄₉₋₅₇ (RAHYNIVTF (SEQ ID NO: 11))-containing tetramers) were measured in peripheral blood (Panels A-C of FIG. 12) or splenocytes (Panels D-F of FIG. 12) either on Days 7, 35, and 63 (Panels A-C of FIG. 12) or Day 42 (Panels D-F of FIG. 12).

Mice were given 10 μg soluble HPV-16 E7 with 10 μg polyIC (Panel A of FIG. 12), 10 μg HPV-16 E7 in ICMVs (E7 ICMV) with 1 μg TLR-4 agonist (Panel B of FIG. 12), or 10 μg HPV-16 E7 in ICMVs (E7 ICMV) with 1 μg TLR-7 agonist (Panel C of FIG. 12). For comparison, data from Vaccine 19:3652-3660, 2001 is shown (Panels D-F of FIG. 12). Mice were given 200 μg TA-CIN (a recombinant HPV-16 L2E6E7 fusion protein vaccine) on Days 0 and 21 (A), 200 μg TA-CIN on Day 0 and 5×10⁶ TA-CIN-expressing vaccinia virus on Day 21 (E), or 5×10⁶ TA-CIN-expressing vaccinia virus on Days 0 and 21 (Panel F of FIG. 12).

FIG. 12 shows that ICMVs including E7 induced the highest percentage of tetramer positive (H2-D^{b} E7₄₉₋₅₇ (RAHYNIVTF (SEQ ID NO: 11))-containing tetramers) cells among CD8-positive T cells compared to that induced by E7 or L2E6E7 fusion protein vaccine.

### Example 21. Tumor Regression Comparison to Minimal Peptide Vaccination

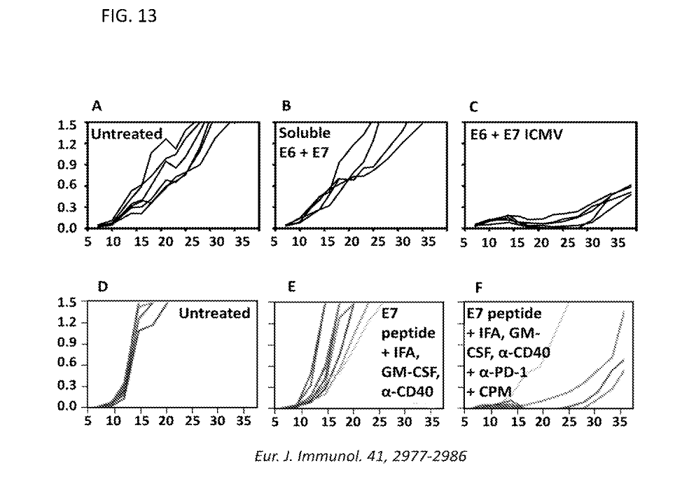

Mice were inoculated on the left dorsal flank with (Panels A-C of FIG. 13) 250,000 or (Panels D-F of FIG. 13) 50,000 TC-1 cells on Day 0 and tumor volume (cm³) was monitored over time.

Mice were either left untreated (Panel A of FIG. 13), or immunized on Days 6, 20, and 34 with 5 μg HPV-16 E6 and 5 μg HPV-16 E7 soluble proteins (Panel B of FIG. 13) or in ICMVs (Panel C of FIG. 13). For comparison, data from Eur. J. Immunol. 41:2977-2986, 2011 is shown (Panels D-F of FIG. 13). Mice were either left untreated (Panel D of FIG. 13) or treated on Days 8, 15, and 22 with 100 μg HPV-16 E7₄₉₋₅₇ (RAHYNIVTF (SEQ ID NO: 11)) in 50 μL incomplete Freund's adjuvant (IFA), together with 5 μg GM-CSF and 20 μg α-CD-40 (Panel E of FIG. 13), or 100 μg HPV-16 E7₄₉₋₅₇ (RAHYNIVTF (SEQ ID NO: 11)) in 50 μL incomplete Freund's adjuvant (IFA), together with 5 μg GM-CSF, 20 μg α-CD-40, 2.5 mg/kg dose of an anti-PD-1 antibody given intravenously (i.v.), and 1 mg cyclophosphamide (CPM) given intraperitoneally (i.p.) (Panel F of FIG. 13). FIG. 13 shows the quantification of tumor volume measured using the equation V=L×W²/2. Mice treated with E6 and E7 in ICMVs (Panel D of FIG. 13) appeared to have the smallest tumor volume.

### Example 22. Tumor Regression Comparison to Listeria Vaccination

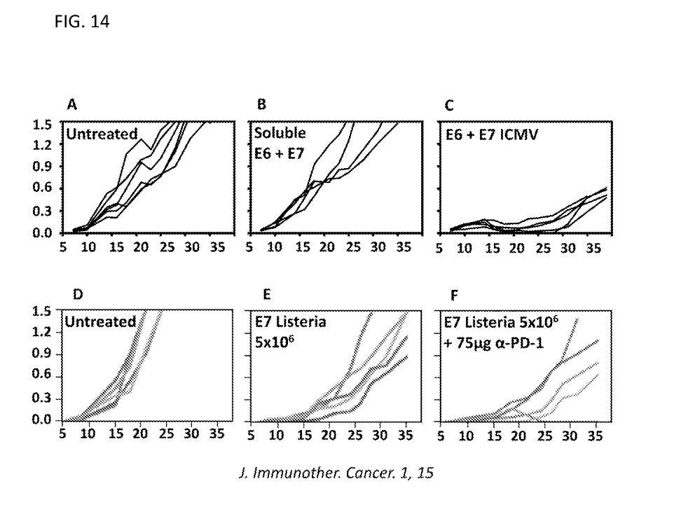

Mice were inoculated on the left dorsal flank with (A-C of FIG. 14) 250,000 or (D-F of FIG. 14) 50,000 TC-1 cells on Day 0 and tumor volume (cm³) was monitored over time.

Mice were either left untreated (A of FIG. 14), or immunized on Days 6, 20, and 34 with 5 μg HPV-16 E6 and 5 μg HPV-16 E7 soluble proteins (B of FIG. 14) or in ICMVs (C of FIG. 14). For comparison, data from J Immunother. Cancer. 1:15, 2013 is shown (D-F of FIG. 14). Mice were either left untreated (D of FIG. 14) or treated on Days 8 and 15 with 5×10⁶ Listeria monocytogenes expressing HPV-16 E7 either alone (E) or together with 2.5 mg/kg or 50 μg of an anti-PD-1 antibody given intravenously (i.v.) (F). FIG. 14 shows the quantification of tumor volume measured using the equation V=L×W²/2. Mice treated with E6 and E7 in ICMVs (D of FIG. 14) appeared to have the smallest tumor volume.

### Example 23. Treatment of Solid Tumors in Mice Using E6-ICMV and E7-ICMV with and without the Immunomodulator Anti-CD40 Antibody

ICMVs including an E6 or E7 protein of HPV-16 (E6-ICMV and E7-ICMV) were tested for their effectiveness in treating a mouse model for solid tumors with and without anti-CD40 antibody. 50,000 tissue culture-1 (TC-1) tumor cells were injected into the left dorsal flank of C57BL/6 mice on Day 0. On Day 6, Day 13, and Day 20, mice were treated with 1) 200 μg anti-CD40 antibody (intraperitoneal (i.p.) injection), 2) 10 μg E7-ICMV+10 μg E6-ICMV (subcutaneous injection), or 3) E6-ICMV+E7-ICMV+anti-CD40 antibody.

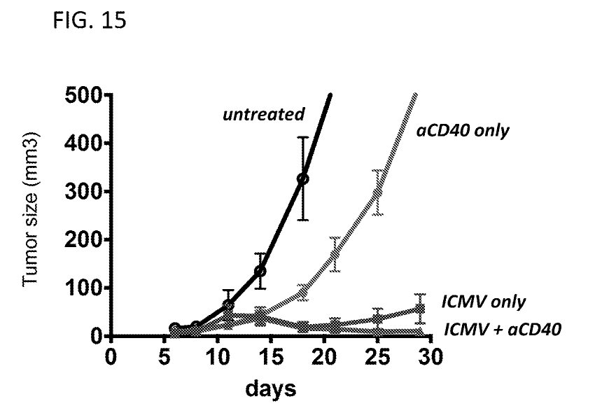

FIG. 15 shows the quantification of tumor volume measured using the equation V=L×W²/2. Mice treated with E6-ICMV+E7-ICMV+anti-CD40 antibody have the smallest tumor volume.

### Example 24. Treatment of Solid Tumors in Mice Using E6-ICMV and E7-ICMV with and without the Immunomodulators Anti-PD-1 Antibody and Cyclophosphamide (CPM)

ICMVs including an E6 or E7 protein of HPV-16 (E6-ICMV and E7-ICMV) were tested for their effectiveness in treating a mouse model for solid tumors with and without anti-PD-1 antibody and cyclophosphamide (CPM). 50,000 tissue culture-1 (TC-1) tumor cells were injected into the left dorsal flank of C57BL/6 mice on Day 0. On Day 6, Day 13, and Day 20, mice were treated with 1) 230 μg anti-PD-1 antibody only (i.p. injection), 2) 10 μg E7-ICMV+10 μg E6-ICMV (subcutaneous injection), 3) E6-ICMV+E7-ICMV+anti-PD-1 antibody, or 4) E6-ICMV+E7-ICMV+anti-PD-1 antibody+cyclophosphamide. Cyclophosphamide (CPM) (1 mg, i.p. injection) was given one day prior to injection of ICMVs and anti-PD-1 antibody.

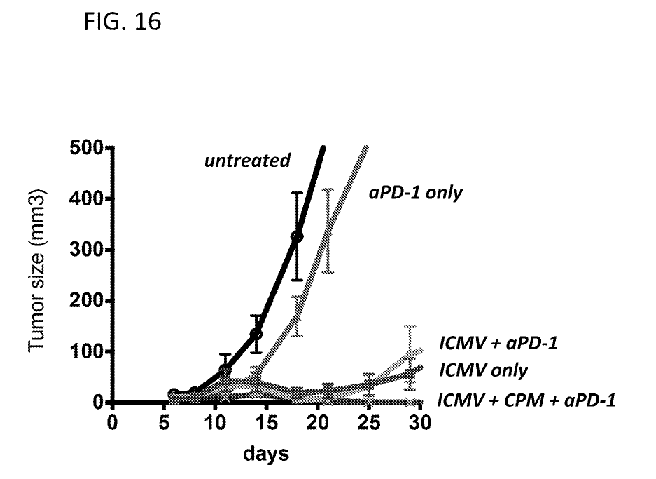

FIG. 16 shows the quantification of tumor volume measured using the equation V=L×W²/2. Mice treated with E6-ICMV+E7-ICMV+anti-PD-1 antibody+cyclophosphamide (CPM) have the smallest tumor volume and 100% cures on Day 30.

### Example 25. Immunogenicity Induced by E6-ICMV and E7-ICMV with and without the Immunomodulators Anti-PD-1 Antibody and AMD3100

Immunogenicity induced by E6-ICMV and E7-ICMV with and without anti-PD-1 antibody and AMD3100 was measured by quantifying the percentage of IFNγ secreting cells among E6/E7-specific CD8 T cells.

50,000 tissue culture-1 (TC-1) tumor cells were injected into the left dorsal flank of C57BL/6 mice on Day 0. On Day 6 and Day 13, mice were treated with 1) 230 μg anti-PD-1 antibody (i.p. injection), 2) 10 μg E7-ICMV+10 μg E6-ICMV (subcutaneous injection), 3) E6-ICMV+E7-ICMV+anti-PD-1 antibody, or 4) E6-ICMV+E7-ICMV+anti-PD-1 antibody+AMD3100. AMD3100 (25 μg, i.p. injection) was injected daily from Day 6 to Day 19.

On Day 19, immunogenicity was tested by quantifying the percentage of IFNγ secreting cells among E6/E7-specific CD8 T cells. Blood cells were collected and stimulated with E749-57 (RAHYNIVTF (SEQ ID NO: 11)) peptide or E648-57 (EVYDFAFRDL (SEQ ID NO: 12)) peptide. The percentage of IFNγ secretion was measured using an intracellular cytokine staining assay.

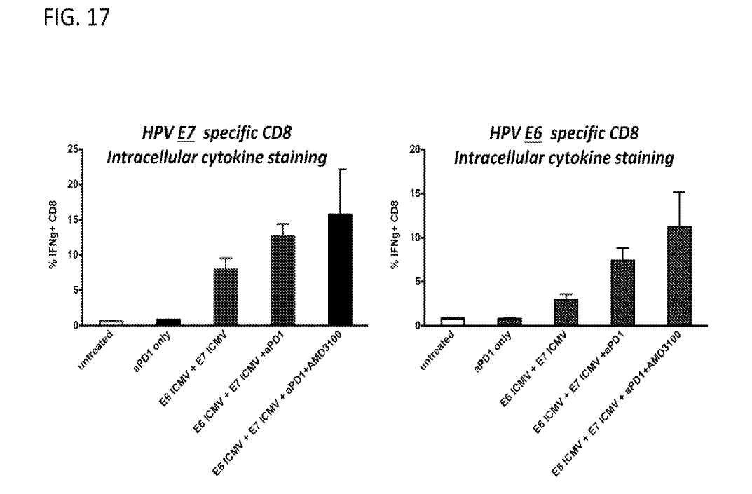

FIG. 17 shows that combination treatment with E6-ICMV+E7-ICMV+anti-PD-1 antibody+AMD3100 produced the highest percentage of IFNγ secreting cells among E6/E7-specific CD8 T cells.

### Other Embodiments

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

What is claimed is:

1. A composition comprising:
(a) a multilamellar lipid vesicle having crosslinks between lipid bilayers; and
(b) one or more mutant human papilloma virus (HPV) peptides;
wherein at least one of said one or more mutant HPV peptides is conjugated to a lipid of said multilamellar lipid vesicle.

2. The composition of claim 1, wherein said mutant HPV peptide has at least one amino acid substitution relative a wild-type HPV peptide sequence.

3. The composition of claim 1 or 2, wherein said mutant HPV peptide has two to six amino acid substitutions relative a wild-type HPV peptide sequence.

4. The composition of any one of claims 1-3, wherein said mutant HPV peptide is derived from a HPV of genotype 16 (HPV-16) or 18 (HPV-18).

5. The composition of any one of claims 1-4, wherein said mutant HPV peptide is derived from a HPV E6 protein or a HPV E7 protein.

6. The composition of any one of claims 1-5, wherein said mutant HPV peptide is derived from a HPV-16 E6 protein.

7. The composition of any one of claims 1-5, wherein said mutant HPV peptide is derived from a HPV-16 E7 protein.

8. The composition of any one of claims 1-5, wherein said mutant HPV peptide is derived from a HPV-18 E6 protein.

9. The composition of any one of claims 1-5, wherein said mutant HPV peptide is derived from a HPV-18 E7 protein.

10. The composition of any one of claims 1-4, wherein said mutant HPV peptide comprises a fusion protein of a first HPV peptide and a second HPV peptide.

11. The composition of claim 10, wherein said first HPV peptide and said second HPV peptide are of the same genotypes.

12. The composition of claim 10, wherein said first HPV peptide and said second HPV peptide are of different genotypes.

13. The composition of claim 10 or 11, wherein said first HPV peptide and said second HPV peptide are both HPV-16 peptides.

14. The composition of claim 13, wherein said HPV-16 peptides are HPV-16 E6 peptides or HPV-16 E7 peptides.

15. The composition of claim 10 or 11, wherein said first HPV peptide and said second HPV peptide are both HPV-18 peptides.

16. The composition of claim 15, wherein said HPV-18 peptides are HPV-18 E6 peptides or HPV-18 E7 peptides.

17. The composition of claim 10 or 12, wherein said first HPV peptide is a HPV-16 peptide and said second HPV peptide is a HPV-18 peptide.

18. The composition of claim 17, wherein said HPV-16 peptide is a HPV-16 E6 or HPV-16 E7 peptide and wherein said HPV-18 peptide is a HPV-18 E6 or HPV-18 E7 peptide.

19. The composition of any one of claims 10-18, wherein said HPV-16 E6 peptide is derived from a HPV-16 E6 protein and wherein said HPV-18 E6 peptide is derived from a HPV-18 E6 protein.

20. The composition of any one of claims 10-18, wherein said HPV-16 E7 peptide is derived from a HPV-16 E7 protein and wherein said HPV-18 E7 peptide is derived from a HPV-18 E7 protein.

21. A composition comprising:
a multilamellar lipid vesicle having crosslinks between lipid bilayers comprising one or more first mutant HPV peptides, and one or more second mutant HPV peptides,
wherein at least one of said first mutant HPV peptides is conjugated to a first lipid of said multilamellar lipid vesicle,
wherein at least one of said second mutant HPV peptides is conjugated to a second lipid of said multilamellar lipid vesicle, and
wherein said first and second mutant HPV peptides are different.

22. The composition of claim 21, wherein said first and/or second mutant HPV peptides are derived from a HPV-16 E6 protein, a HPV-16 E7 protein, a HPV-18 E6 protein, or a HPV-18 E7 protein.

23. The composition of any one of claims 6, 19, and 22, wherein said HPV-16 E6 protein has the sequence of SEQ ID NO: 1.

24. The composition of any one of claims 7, 20, and 22, wherein said HPV-16 E7 protein has the sequence of SEQ ID NO: 2.

25. The composition of any one of claims 8, 19, and 22, wherein said HPV-18 E6 protein has the sequence of SEQ ID NO: 3.

26. The composition of any one of claims 9, 20, and 22, wherein said HPV-18 E7 protein has the sequence of SEQ ID NO: 4.

27. The composition of any one of claims 1-26, wherein said one or more mutant HPV peptides comprise one or more functionalized mutant HPV peptides.

28. The composition of claim 27, wherein said one or more functionalized mutant HPV peptides comprise thiol functionalized, maleimide functionalized, hydrazine functionalized, azide functionalized, alkyne functionalized, amine functionalized, carboxylic acid functionalized, alkene functionalized, and/or tetrazine functionalized mutant HPV peptides.

29. The composition of claim 28, wherein said one or more functionalized mutant HPV peptides are maleimide functionalized and/or hydrazine functionalized.

30. The composition of any one of claims 1-29, wherein said conjugated mutant HPV peptide is encapsulated within the vesicle.

31. The composition of any one of claims 1-29, wherein said conjugated mutant HPV peptide is encapsulated between lipid bilayers of said vesicle.

32. The composition of any one of claims 1-29, wherein said conjugated mutant HPV peptide is present on an outer surface of the vesicle.

33. The composition of any one of claims 1-32, wherein said composition comprises at least one molecule of said one or more mutant HPV peptides that is not conjugated to a lipid of said multilamellar lipid vesicle.

34. The composition of any one of claims 1-33, further comprising an immunomodulator.

35. The composition of anyone of claims 1-34, further comprising an adjuvant.

36. A composition comprising:
(a) a multilamellar lipid vesicle having crosslinks between lipid bilayers; and
(b) one or more agents;
wherein said one or more agents are not terminal-cysteine-bearing antigens; and
wherein at least one molecule of said one or more agents is conjugated to a lipid of said multilamellar lipid vesicle.

37. The composition of claim 36, wherein said agent comprises an internal cysteine.

38. A composition comprising:
(a) a multilamellar lipid vesicle having crosslinks between lipid bilayers; and
(b) one or more agents;
wherein at least one molecule of said one or more agents is conjugated to a lipid of said multilamellar lipid vesicle, and
wherein said conjugation does not comprise a cysteine.

39. A composition comprising:
(a) a multilamellar lipid vesicle having crosslinks between lipid bilayers; and
(b) one or more agents;
wherein at least one molecule of said one or more agents is conjugated to a lipophilic moiety.

40. The composition of claim 39, wherein said lipophilic moiety comprises an acyl group.

41. The composition of any one of claims 36-40, further comprising an adjuvant.

42. The composition of any one of claims 36-41, further comprising an immunomodulator.

43. The composition of any one of claims 36-42, wherein said agent is an adjuvant.

44. The composition of any one of claims 36-42, wherein said one or more agents comprise one or more antigens.

45. The composition of any one of claims 36-42 and 44, wherein said one or more agents comprise two or more antigens.

46. The composition of claim 44 or 45, wherein said antigen is a full-length protein antigen.

47. The composition of claim 44 or 45, wherein said antigen is a peptide antigen.

48. The composition of any one of claims 44-47, wherein said antigen is a cancer antigen.

49. The composition of claim 48, wherein said cancer antigen is a gp100.

50. The composition of claim 48, wherein said cancer antigen is a NY-ESO-1.

51. The composition of claim 48, wherein said cancer antigen is a member of the mucin (MUC) family.

52. The composition of claim 48, wherein said cancer antigen is MUC1.

53. The composition of claim 48, wherein said cancer antigen is selected from a group consisting of a MAGE-A1, a MAGE-A2, a MAGE-A3, a MAGE-A4, a MAGE-A5, a MAGE-A6, a MAGE-A7, a MAGE-A8, a MAGE-A9, a MAGE-A10, a MAGE-A11, a MAGE-A12, a MAGE-Xp2 (MAGE-B2), a MAGE-Xp3 (MAGE-B3), a MAGE-Xp4 (MAGE-B4), a MAGE-C1, a MAGE-C2, a MAGE-C3, a MAGE-C4, a MAGE-05, a WT1 (Wilms' tumor antigen 1), a MUC1, a LMP2 (latent membrane protein 2 from Epstein-Barr virus), an EGFRvIII, a Her2/neu, Idiotype antigens, a non-mutant p53, a NY-ESO-1, a PSMA (prostate-specific membrane antigen), a GD2, a CEA (carcinoembryonic antigen), a MelanA/MART1, Ras mutants, a gp100, a mutant p53, a Proteinase3 (PR1), BCR-Abl breakpoints, a Tyrosinase, a Survivin, a PSA (prostate-specific antigen), a hTERT (human telomerase), sarcoma translocation breakpoints, an EphA2, a PAP (prostatic acid phosphatase), a ML-IAP (ML-inhibitor of apoptosis), an AFP (alphafetoprotein), an EpCAM (epithelial cell adhesion molecule), an ERG (TMPRSS2 ETS fusion gene), a NA17, a PAX3 (paired box 3), an Androgen receptor, a Cyclin B1, a Polysialic acid, a MYCN (N-myc), a RhoC, a TRP-2 (tyrosinase-related protein 2), a GD3, a Fucosyl GM1, a Mesothelin, a PSCA (prostate stem cell antigen), a CYP1B1 (cytochrome P450 1B1), a PLAC1 (placenta-specific 1), a GM3, a BORIS (brother of the regulator of imprinted sites), a Tn (N-acetylgalactosamine linked to serine or threonine by a glycosidic bond), a GloboH, an ETV6-AML, a NY-BR-1, a RGS5 (regulator of G protein signaling 5), a SART3 (squamous cell carcinoma antigen recognized by T cells 3), a STn (sialyl Tn antigen), a Carbonic anhydrase IX, PAX5 (paired box 5), an OY-TES1, a Sperm protein 17, a LCK (p56 form), a HMWMAA (high molecular weight melanoma associated antigen), an AKAP-4 (A-kinase anchor protein 4), a SSX2 (synovial sarcoma breakpoint 2), a XAGE1 (x antigen 1), a B7H3, a Legumain, a Tie 2, a Page4, a VEGFR2 (vascular endothelial growth factor receptor 2), a MAD-CT-1 (melanoma cancer testis antigen-1), a FAP (fibroblast activation protein), a PDGFR-b (platelet-derived growth factor receptor-b), a MAD-CT-2 (melanoma cancer testis antigen-2), and a Fos-related antigen 1.

54. The composition of any one of claims 36-40, wherein said agent is a HPV peptide.

55. The composition of claim 54, wherein said HPV peptide is derived from a HPV-16 E6 protein, a HPV-16 E7 protein, a HPV-18 E6 protein, or a HPV-18 E7 protein.

56. The composition of claim 54, wherein said HPV peptide comprises a fusion protein of a first HPV peptide and a second HPV peptide.

57. The composition of claim 56, wherein said first HPV peptide and said second HPV peptide are of the same genotypes.

58. The composition of claim 56, wherein said first HPV peptide and said second HPV peptide are of different genotypes.

59. The composition of claim 56 or 57, wherein said first HPV peptide and said second HPV peptide are both HPV-16 peptides.

60. The composition of claim 59, wherein said HPV-16 peptides are HPV-16 E6 peptides or HPV-16 E7 peptides.

61. The composition of claim 56 or 57, wherein said first HPV peptide and said second HPV peptide are both HPV-18 peptides.

62. The composition of claim 61, wherein said HPV-18 peptides are HPV-18 E6 peptides or HPV-18 E7 peptides.

63. The composition of claim 56 or 58, wherein said first HPV peptide is a HPV-16 peptide and said second HPV peptide is a HPV-18 peptide.

64. The composition of claim 63, wherein said HPV-16 peptide is a HPV-16 E6 or HPV-16 E7 peptide and wherein said HPV-18 peptide is a HPV-18 E6 or HPV-18 E7 peptide.

65. The composition of any one of claims 56-64, wherein said HPV-16 E6 peptide is derived from a HPV-16 E6 protein and wherein said HPV-18 E6 peptide is derived from a HPV-18 E6 protein.

66. The composition of any one of claims 56-64, wherein said HPV-16 E7 peptide is derived from a HPV-16 E7 protein and wherein said HPV-18 E7 peptide is derived from a HPV-18 E7 protein.

67. The composition of any one of claims 36-66, wherein said one or more agents comprise one or more functionalized agents.

68. The composition of claim 67, wherein said one or more functionalized agents comprise thiol functionalized, maleimide functionalized, hydrazine functionalized, azide functionalized, alkyne functionalized, amine functionalized, carboxylic acid functionalized, alkene functionalized, and/or tetrazine functionalized agents.

69. The composition of claim 68, wherein said one or more functionalized agents are maleimide functionalized and/or hydrazine functionalized.

70. The composition of any one of claims 36-69, wherein said conjugated agent is encapsulated within the vesicle.

71. The composition of any one of claims 36-69, wherein said conjugated agent is encapsulated between lipid bilayers of said vesicle.

72. The composition of any one of claims 36-69, wherein said conjugated agent is present on an outer surface of the vesicle.

73. The composition of any one of claims 36-72, wherein said composition comprises at least one molecule of said one or more agents that is not conjugated to a lipid.

74. The composition of any one of claims 1-73, wherein at least two lipid bilayers in the multilamellar lipid vesicle are covalently crosslinked to each other through headgroups that react with covalent crosslinkers to form the covalent crosslinks between lipid bilayers.

75. The composition of any one of claims 1-74, wherein said lipid bilayers comprise anionic and/or neutral lipids.

76. The composition of any one of claims 1-75, wherein said lipid bilayers comprise cationic lipids.

77. The composition of any one of claims 1-76, wherein said vesicle comprises a functionalized lipid.

78. The composition of claim 77, wherein said functionalized lipid is a maleimide functionalized lipid.

79. The composition of claim 78, wherein said maleimide functionalized lipid is a maleimide functionalized phosphoethanolamine.

80. The composition of any one of claims 1-79, wherein said vesicle comprises phosphocholine.

81. The composition of any one of claims 1-80, wherein said vesicle comprises phosphoglycerol.

82. The composition of any one of claims 1-81, wherein said vesicle comprises dioleoylphosphatidylcholine (DOPC), dioleoylphosphatidylglycerol (DOPG), and a maleimide functionalized lipid.

83. The composition of any one of claims 1-82, wherein said vesicle is conjugated to polyethylene glycol.

84. The composition of any one of claims 1-83, further comprising one or more pharmaceutically acceptable carriers.

85. The composition of any one of claims 1-84, further comprising an excipient suitable for lyophilization.

86. The composition of claim 85, wherein the excipient suitable for lyophilization comprises sucrose.

87. A method comprising administering to a subject in need thereof, a therapeutically effective amount of a composition of any one of claims 1-86.

88. The method of claim 87, wherein an immunomodulator is not administered to said subject.

89. A method comprising administering to a subject in need thereof, 1) a therapeutically effective amount of a composition of any one of claims 1-86, and 2) one or more immunomodulators, wherein said immunomodulator is not a CT-011 antibody.

90. A method comprising administering to a subject in need thereof, 1) a multilamellar lipid vesicle having crosslinks between lipid bilayers comprising one or more mutant HPV peptides, and 2) one or more immunomodulators, wherein said immunomodulator is not a CT-011 antibody.

91. The method of claim 89 or 90, wherein said 1) and 2) are administered substantially simultaneously.

92. The method of claim 89 or 90, wherein said 1) and 2) are administered separately.

93. The method of claim 92, wherein said 1) is administered first, followed by administering of 2).

94. The method of claim 92, wherein said 2) is administered first, followed by administering of 1).

95. The method of claim 89 or 90, wherein said immunomodulator is selected from the group consisting of a PD-1 inhibitor, an anti-CTLA-4 antibody, an anti-CD40 antibody, a cyclophosphamide (CPM), an AMD3100, an anti-LAG-3/CD223 antibody, an anti-B7-H5 antibody, an anti-OX40 antibody, an anti-CD28 antibody, an anti-GITR antibody, an anti-4-1 BB/CD137 antibody, a 4-1 BB ligand, an anti-BTLA antibody, an anti-TIM-3/HAVCR2 antibody, an anti-KIR antibody, an anti-Flt3/CD135 antibody, an anti-FasL antibody, an anti-CD25 antibody, an GM-CSF, an anti-GM-CSF-receptor (R) antibody, an IL-2, an anti-IL-2-R antibody, an IL-7, an anti-IL-7-R antibody, an IL-21, an anti-IL-21-R antibody, an IL-12, an anti-IL-12-R antibody, an IL-15, an anti-IL-15-R antibody, an IL-18, an anti-IL-18-R antibody, an anti-IDO antibody, an ipilimumab, a crizotinib, a ceritinib, a celecoxib, a SOCS-1 inhibitor, a heat shock protein (HSP), a HSP inhibitor, and an anti-galectin-1 antibody.

96. The method of claim 95, wherein said immunomodulator is selected from the group consisting of a PD-1 inhibitor, an anti-GITR antibody, an anti-CTLA-4 antibody, an anti-CD40 antibody, a cyclophosphamide (CPM), and an AMD3100.

97. The method of claim 96, wherein said PD-1 inhibitor is an anti-PD-1 antibody or an anti-PD-L1 antibody.

98. The method of any one of claims 87-97, wherein said subject has cancer.

99. The method of claim 98, wherein said cancer is HPV-positive.

100. The method of claim 98 or 99, wherein said cancer is selected from cervical cancer, anal cancer, vulvar cancer, head and neck cancer, oropharyngeal cancer, penile cancer, vaginal cancer, virally induced cancer, bladder cancer, pancreatic cancer, lung cancer, liver cancer, ovarian cancer, colon cancer, stomach cancer, neuroblastoma, breast cancer, prostate cancer, renal cancer, leukemia, sarcoma, carcinoma, basal cell carcinoma, non-small cell lung carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, sarcoma, melanoma, astrocytoma, oligoastrocytoma, biliary tract cancer, choriocarcinoma, CNS cancer, larynx cancer, small cell lung cancer, non-small cell lung cancer (NSCLC), adenocarcinoma, giant (or oat) cell carcinoma, squamous cell carcinoma, oral cavity cancer, skin cancer, basal cell cancer, squamous cell cancer, testicular cancer, thyroid cancer, uterine cancer, rectal cancer, a cancer of the respiratory system, a cancer of the urinary system, a cancer of the digestive system, bone cancer, brain cancer, colorectal cancer, connective tissue cancer, endometrial cancer, eye cancer, gastric cancer, intra-epithelial neoplasm, melanoma neuroblastoma, retinoblastoma, and rhabdomyosarcoma.

101. The method of claim 100, wherein said cancer is a cervical cancer.

102. The method of claim 100, wherein said cancer is an anal cancer.

103. The method of claim 100, wherein said cancer is a vulvar cancer.

104. The method of claim 100, wherein said cancer is a head and neck cancer.

105. The method of claim 100, wherein said cancer is an oropharyngeal cancer.

106. The method of claim 100, wherein said cancer is a penile cancer.

107. The method of claim 100, wherein said cancer is a vaginal cancer.

108. The method of any one of claims 98-107, wherein said cancer is a solid tumor cancer.

109. The method of any one of claims 87-97, wherein said subject has an infection.

110. The method of claim 109, wherein said infection is a HPV infection.

111. The method of any one of claims 87-110, wherein said subject is a mammal.

112. The method of claim 111, wherein said mammal is a human.

113. A method comprising
(a) contacting a functionalized lipid with a functionalized agent to form liposomes comprising said lipid conjugated to said agent;
(b) contacting said liposomes comprising lipids conjugated to said agent with a divalent cation to form fused liposomes; and
(c) contacting said fused liposomes with a crosslinker to form multilamellar lipid vesicles having crosslinks between lipid bilayers comprising lipids conjugated to said agent,
wherein said functionalized agent is not a terminal-cysteine-bearing antigen.

114. A kit comprising:
(i) a multilamellar lipid vesicle having crosslinks between lipid bilayers comprising one or more agents,
(ii) one or more immunomodulators, and
(iii) instructions for administering (i) and (ii) to a subject having a disease,
wherein said agent is not a terminal-cysteine-bearing antigen, and
wherein said immunomodulator is not a CT-011 antibody.

115. A kit comprising:
(i) a multilamellar lipid vesicle having crosslinks between lipid bilayers comprising one or more agents, and
(ii) instructions for administering (i) with one or more immunomodulators to a subject having a disease,
wherein said agent is not a terminal-cysteine-bearing antigen, and
wherein said immunomodulator is not a CT-011 antibody.

116. A kit comprising:
(i) one or more immunomodulators, and
(ii) instructions for administering (i) with a multilamellar lipid vesicle having crosslinks between lipid bilayers comprising one or more agents to a subject having a disease,
wherein said agent is not a terminal-cysteine-bearing antigen, and
wherein said immunomodulator is not a CT-011 antibody.

117. The kit of any one of claims 114-116, wherein said agent is a mutant HPV peptide.

118. The kit of claim 117, wherein said mutant HPV peptide is derived from a HPV-16 E6 protein, a HPV-16 E7 protein, a HPV-18 E6 protein, or a HPV-18 E7 protein.

119. The kit of claim 117, wherein said mutant HPV peptide comprises a fusion protein of a first HPV peptide and a second HPV peptide.

120. The kit of claim 119, wherein said first HPV peptide and said second HPV peptide are of the same genotypes.

121. The kit of claim 119, wherein said first HPV peptide and said second HPV peptide are of different genotypes.

122. The kit of claim 119 or 120, wherein said first HPV peptide and said second HPV peptide are both HPV-16 peptides.

123. The kit of claim 122, wherein said HPV-16 peptides are HPV-16 E6 peptides or HPV-16 E7 peptides.

124. The kit of claim 119 or 120, wherein said first HPV peptide and said second HPV peptide are both HPV-18 peptides.

125. The kit of claim 124, wherein said HPV-18 peptides are HPV-18 E6 peptides or HPV-18 E7 peptides.

126. The kit of claim 119 or 121, wherein said first HPV peptide is a HPV-16 peptide and said second HPV peptide is a HPV-18 peptide.

127. The kit of claim 126, wherein said HPV-16 peptide is a HPV-16 E6 or HPV-16 E7 peptide and wherein said HPV-18 peptide is a HPV-18 E6 or HPV-18 E7 peptide.

128. The kit of any one of claims 119-127, wherein said HPV-16 E6 peptide is derived from a HPV-16 E6 protein and wherein said HPV-18 E6 peptide is derived from a HPV-18 E6 protein.

129. The kit of any one of claims 119-127, wherein said HPV-16 E7 peptide is derived from a HPV-16 E7 protein and wherein said HPV-18 E7 peptide is derived from a HPV-18 E7 protein.

130. The kit of any one of claims 114-129, wherein said disease is cancer.

131. The kit of claim 130, wherein said cancer is HPV-positive.

132. The kit of claim 130 or 131, wherein said cancer is selected from cervical cancer, anal cancer, vulvar cancer, head and neck cancer, oropharyngeal cancer, penile cancer, vaginal cancer, virally induced cancer, bladder cancer, pancreatic cancer, lung cancer, liver cancer, ovarian cancer, colon cancer, stomach cancer, neuroblastoma, breast cancer, prostate cancer, renal cancer, leukemia, sarcoma, carcinoma, basal cell carcinoma, non-small cell lung carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, sarcoma, melanoma, astrocytoma, oligoastrocytoma, biliary tract cancer, choriocarcinoma, CNS cancer, larynx cancer, small cell lung cancer, non-small cell lung cancer (NSCLC), adenocarcinoma, giant (or oat) cell carcinoma, squamous cell carcinoma, oral cavity cancer, skin cancer, basal cell cancer, squamous cell cancer, testicular cancer, thyroid cancer, uterine cancer, rectal cancer, a cancer of the respiratory system, and a cancer of the urinary system.

133. The kit of any one of claims 114-129, wherein said disease is infection.

134. The kit of claim 133, wherein said infection is a HPV infection.

135. The kit of any one of claims 114-134, wherein said subject is a mammal.

136. The kit of claim 135, wherein said mammal is a human.


SEQUENCE LISTING (12 sequences)

SEQ ID NO: 1 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 2 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 3 - protein, 145 aa, Human papillomavirus type 18
LPDLCTELNTSLQDIEITCVYCKTVLELTEVFEFAFKDLFVVYRDSIPHAACHKCIDFYSRIRELRYYSDSVYGDTLEKLTNTGLYNLLIRCLRCQKPLNPAEKLRHLNEKRRFHKIAGHYRGQCHSCCNRARQERLQRRRETQV

SEQ ID NO: 4 - protein, 105 aa, Human papillomavirus type 18
MYGPKATLQDIVLHLEPQNEIPVDLLCHEQLSDSEEENDEIDGVNHQHLPARRAEPQRHTMLCMCCKCEARIELVVESSADDLRAFQQLFLSTLSFVCPWCASQQ

SEQ ID NO: 5 - protein, 158 aa, Artificial Sequence
  Synthetic Construct
MHQKRTAMFQDPQERPRKLPQLCTELQTTIHDIILECVYCKQQLLRREVYDFAFRDLCIVYRDGNPYAVGDKCLKFYSKISEYRHYCYSLYGTTLEQQYNKPLCDLLIRCINGQKPLCPEEKQRHLDKKQRFHNGRGRWTGRCMSCCRSSRTRRETQL

SEQ ID NO: 6 - protein, 98 aa, Artificial Sequence
  Synthetic Construct
MHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 7 - protein, 145 aa, Artificial Sequence
  Synthetic Construct
LPDLCTELNTSLQDIEITCVYCKTVLELTEVFEFAFKDLFVVYRDSIPHAAGHKCIDFYSRIRELRYYSDSVYGDTLEKLTNTGLYNLLIRCLRGQKPLNPAEKLRHLNEKRRFHKGAGHYRGQCHSCCNRARQERLQRRRETQV

SEQ ID NO: 8 - protein, 158 aa, Artificial Sequence
  Synthetic Construct
MARFEDPTRRPYKLPDLCTELNTSLQDIEITCVYCKTVLELTEVFEFAFKDLFVVYRDSIPHAAGHKCIDFYSRIRELRHYSDSVYGDTLEKLTNTGLYNLLIRCLRGQKPLNPAEKLRHLNEKRRFHNGAGHYRGQCHSCCNRARQERLQRRRETQV

SEQ ID NO: 9 - protein, 105 aa, Artificial Sequence
  Synthetic Construct
MHGPKATLQDIVLHLEPQNEIPVDLLGHGQLSDSEEENDEIDGVNHQHLPARRAEPQRHTMLCMCCKCEARIKLVVESSADDLRAFQQLFLNTLSFVCPWCASQQ

SEQ ID NO: 10 - protein, 256 aa, Artificial Sequence
  Synthetic Construct
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 11 - protein, 9 aa, Artificial Sequence
  Synthetic Construct
RAHYNIVTF

SEQ ID NO: 12 - protein, 10 aa, Artificial Sequence
  Synthetic Construct
EVYDFAFRDL